(19) United States
(12) Patent Application Publication
(10) Pub. No.: US 2011/0160440 A1
(43) Pub. Date: Jun. 30, 2011

(54) Cab Molecules

(75) Inventors: Judith A. Fox, San Francisco, CA (US); Fiona A. Harding, Santa Clara, CA (US); Volker Schellenberger, Palo Alto, CA (US)

(73) Assignee: GENENCOR INTERNATIONAL, INC., Palo Alto, CA (US)

(21) Appl. No.: 10/581,106

(22) PCT Filed: Dec. 10, 2004
(86) PCT No.: PCT/US2004/041429

§ 371 Date: Sep. 10, 2007

Related U.S. Application Data
(60) Provisional application No. 60/529,354, filed on Dec. 12, 2003.
(60) Provisional application No. 60/577,255, filed on Jun. 4, 2004.

(51) Int. Cl. C07K 16/30; C07H 21/04

(52) U.S. Cl. 530/391.1; 536/23.5

(57) ABSTRACT

The present invention relates to CAB molecules, ADEPT constructs directed against CEA, and their use in diagnosis and therapy.

### FIELD OF THE INVENTION

The present invention relates to CAB molecules, ADEPT constructs directed against CEA, and their use in diagnosis and therapy.

### BACKGROUND

Traditional therapeutic molecules circulate freely throughout the body of patients, until they are removed from circulation by the liver or another mechanism of clearance. Such non-targeted molecules exert their pharmocological effects indiscriminately on a wide range of cells and tissues. This can cause serious side effects in the patient. The problem is particularly acute when the molecule is a highly toxic chemotherapeutic agent used to kill cancer cells where the therapeutic window, that is the difference between an efficacious dose and an injurious, or even lethal, dose can be small. Thus, in recent years, researchers have attempted to develop compounds that specifically affect particular subsets of cells, tissues or organs in a patient. Most of the compounds target a particular tissue by preferentially binding a particular target molecule displayed by the tissue to be treated. By preferentially affecting targeted cells, tissues or organs, the difference between an efficacious dose and an injurious dose can be increased, which in turn increases the opportunity for a successful treatment regimen and reduces the occurrence of side effects.

One version of an approach that utilizes preferential binding is antibody-directed enzyme prodrug therapy (ADEPT). See, e.g., Xu et al., 2001, Clin Cancer Res. 7:3314-24.; Denny, 2001, Eur J Med Chem. 36:577-95. In ADEPT, an antibody or antibody fragment is linked to an enzyme capable of converting a pro-drug into an active cytotoxic agent. The ADEPT conjugate is administered to the patient, and the conjugate is localized to a target tissue. The prodrug is then subsequently administered to the patient. The prodrug circulates throughout the body of the patient, but causes few or no side effects because it is in its inactive form. The prodrug is converted into its active drug form by the localized ADEPT conjugate's enzyme. Because the ADEPT conjugate is localized to the target tissue, the prodrug is activated only in the vicinity of the target tissue. Thus, a relatively low concentration of active drug is present throughout the body, but a relatively high concentration of active drug is produced in the vicinity of the target tissue, allowing the drug to exert its therapeutic effects at the desired site, increasing the therapeutic window of the toxin.

Carcinoembryonic antigen (“CEA”) was first described by Gold and Freedman, J. Exp. Med., 121, 439-462, (1965). CEA is expressed by most colorectal cancers and by a number of other tumors. CEA is highly expressed in tumor tissue, and it is also found at a lower concentration in normal organs in particularly in the digestive tract.

### SUMMARY OF THE INVENTION

The present invention relates to CAB molecules, ADEPT constructs directed against CEA, and their use in diagnosis and therapy.

In a first aspect, the invention is drawn to a CAB molecule comprising a modified amino acid sequence. In one embodiment, the CAB molecule has the unmodified sequence set forth in SEQ ID NO:1. In one embodiment, the CAB molecule has an amino acid sequence modified from the amino acid sequence set forth in SEQ ID NO:1, and the modification is at least one position selected from the group consisting of positions 100, 102, 104, 105, 107, 163, 165, 166, 184 and 226, wherein position numbering is with respect to SEQ ID NO:1 as shown in FIG. 1. In a preferred embodiment, the CAB molecule comprises modifications at positions 100, 184 and 226. In a preferred embodiment, the CAB molecule comprises modifications at positions 100, 102, 104, 105, 107, 163, 165, 166, 184 and 226. In a preferred embodiment, the CAB molecule comprises modifications at positions 100, 102, 104, 105, 107, 163, 165, 166 and 226.

In a preferred embodiment, the modification is at least one selected from the group consisting of T100L, T102L, P104A, Y105I, F107N, S163A, S165Y, Y166A, S184D and S226D, wherein position numbering is with respect to SEQ ID NO:1 as shown in FIG. 1. In a preferred embodiment, the CAB molecule comprises a CAB1.6 molecule, the CAB1.6 molecule having the following modifications: T100L, S184D and S226D. In a preferred embodiment, the CAB molecule comprises a CAB1.7 molecule, the CAB1.7 molecule having the following modifications: T100L, T102L, P104A, Y105I, F107N, S163A, S165Y, Y166A, S184D and S226D. In a preferred embodiment, the CAB molecule comprises a CAB1.13 molecule, the CAB1.13 molecule having the following modifications: T100L, T102L, P104A, Y105I, F107N, S163A, S165Y, Y166A and S226D.

In a preferred embodiment, the CAB molecule comprises the scFV portion of CAB 1.2 (SEQ ID NO:1), CAB1.6 (SEQ ID NO:5), CAB1.7 (SEQ ID NO:6) or CAB1.13 as set forth in FIG. 25.

In a preferred embodiment, the CAB molecule further comprises a beta-lactamase molecule. In a preferred embodiment, the CAB molecule has an amino acid sequence which is unmodified or modified from the amino acid sequence set forth in SEQ ID NO:2, and the modification is at least one position selected from the group consisting of positions: 3, 13, 16, 37, 100, 102, 104, 105, 107, 146, 163, 165, 166, 181, 184, 226, 265 and 568, wherein position numbering is with respect to SEQ ID NO:2 as shown in FIG. 2. In a preferred embodiment, the modifications are at positions 3, 13, 16, 37, 100, 146, 181, 184 and 226. In a preferred embodiment, the modifications are at positions 3, 13, 16, 37, 100, 102, 104, 105, 107, 146, 163, 165, 166, 181, 184 and 226. In a preferred embodiment, the modifications are at positions 265 and 568. In a preferred embodiment, the modifications are at positions 3, 13, 16, 37, 100, 102, 104, 105, 107, 146, 163, 165, 166, 181, 184, 226, 265 and 568. In a preferred embodiment, the modifications are at 3, 13, 16, 37, 100, 102, 104, 105, 107, 146, 163, 165, 166, 181, 226, 265 and 568.

In a preferred embodiment, the CAB molecule has modifications comprising at least one modification selected from the group consisting of K3Q, R13K, T16G, L37V, T100L, T102L, P104A, Y105I, F107N, M146V, S163A, S165Y, Y166A, W181V, S184D, S226D, K265A and S568A, wherein position numbering is with respect to SEQ ID NO:2 as shown in FIG. 2. In a preferred embodiment, the CAB molecule comprises a CAB1.2i molecule, the CAB 1.2i molecule comprising the following modifications: K265A and S568A. In a preferred embodiment, the CAB molecule comprises a CAB1.6 molecule, the CAB 1.6 molecule comprising the following modifications: K3Q, R13K, T16G, L37V, T100L, M146V, W181V, S184D and S226D. In a preferred embodiment, the CAB molecule comprises a CAB1.6i molecule, the CAB1.6i molecule comprising the following modifications: K3Q, R13K, T16G, L37V, T100L, M146V, W181V, S184D, S226D, K265A and S568A. In a preferred embodiment, the CAB molecule comprises a CAB1.7 molecule, the CAB1.7 molecule comprising the following modifications: K3Q, R13K, T16G, L37V, T100L, T102L, P104A, Y105I, F107N, M146V, S163A, S165Y, Y166A, W181V, S184D and S226D. In a preferred embodiment, the CAB comprises a CAB1.7i molecule, the CAB1.7i molecule comprising the following modifications: K3Q, R13K, T16G, L37V, T100L, T102L, P104A, Y105I, F107N, M146V, S163A, S165Y, Y166A, W181V, S184D, S226D, K265A and S568A. In a preferred embodiment, the CAB molecule comprises a CAB1.13 molecule, the CAB1.13 molecule comprising the following modifications: K3Q, R13K, T16G, L37V, T100L, T102L, P104A, Y105I, F107N, M146V, S163A, S165Y, Y166A, W181V and S226D. In a preferred embodiment, the CAB comprises a CAB1.13i molecule, the CAB1.13i molecule comprising the following modifications: K3Q, R13K, T16G, L37V, T100L, T102L, P104A, Y105I, F107N, M146V, S163A, S165Y, Y166A, W181V, S226D, K265A and S568A.

In a preferred embodiment, the CAB molecule comprises CAB1.2 (SEQ ID NO:2) or CAB1.2i as set forth in FIG. 25, CAB1.6 (SEQ ID NO:7), CAB1.6i (SEQ ID NO:8), CAB1.7 (SEQ ID NO:9), CAB1.7i (SEQ ID NO:10), CAB1.13 as set forth in FIG. 25 or CAB1.13i as set forth in FIG. 25.

In a second aspect, the invention is drawn to a nucleic acid encoding a CAB molecule as set forth herein. In a third aspect, the invention is drawn to treating a subject in need thereof, comprising administering to the subject a CAB molecule, as provided herein, and a prodrug that is a substrate of the CAB molecule. In a fourth aspect, the invention is drawn to a pharmaceutical composition comprising a CAB molecule.

### BRIEF DESCRIPTION OF THE FIGURES

FIG. 1 sets forth the amino acid sequence of the six CDRs of an unmodified CAB molecule. Position numbering starts with the first position of H1, as set forth in SEQ ID NO:2 as shown in FIG. 2A. Position numbering of the 6 CDRs with respect to SEQ ID NO:1, as shown in FIG. 1, is as follows: H1:26-35; H2, 50-65; H3, 99-109; L1, 159-168; L2, 184-190 and L3, 223-231.

FIG. 2 sets forth the amino acid sequence of the CAB1 molecule (2A) and the amino acid sequence for BLA (2B).

FIG. 3 sets forth the amino acid for the CAB1.6 CDR (3A) and the CAB1.7CDR (3B).

FIG. 4 sets forth the amino acid sequence for the CAB 1.6 (4A)_and CAB 1.6i (4B) molecule.

FIG. 5 sets forth the amino acid sequence for the CAB1.7 (5A) and CAB1.7 i (5B) molecule.

FIG. 6 present details related to plasmid pME27.1. FIG. 6A presents a schematic diagram of plasmid pME27.1. P lac=lac promoter, Pel B leader sequence=signal seq, CAB lscFv=single chain antibody, BLA=β-lactamase gene, CAT=chloramphenicol acetyl transferase resistance gene, 17 terminator=terminator. FIG. 6B shows the sequence of CAB1-scFv, the CDRs and mutations chosen for combinatorial mutagenesis. FIG. 6C presents and nucleotide sequence of pME27.1. FIG. 6D shows the amino acid sequence of CAB1 that shows, for example, the sequence of the heavy chain, the sequence of the linker, the sequence of the light chain and the sequence of BLA.

FIG. 7 shows binding assays and SDS PAGE (polyacrylamide gel electrophoresis results. Specifically, FIG. 7A shows the binding of variants from library NA05; FIG. 7B displays and SDS PAGE of stable CAB1-BLA variants of the NA05 library; FIG. 7C shows binding of various isolates from NA06 tO CEA.

FIG. 8 shows a comparison of vH and vL sequences of CAB1-scFv with a published frequency analysis of human antibodies. Specifically, FIG. 8A shows the observed frequencies of the five most abundant amino acids in alignment with the human sequence in the heavy chain; FIG. 8B shows the observed frequencies of the five most abundant amino acids in alignment with the human sequence in the light chain.

FIG. 9 shows screening results of NA08 library. The x-axis shows binding at pH 7.4, and the Y-axis shows binding at pH 6.5. Clones that were chosen for further analysis are represented by a square.

FIG. 10 shows a three dimensional model with positions that were chosen for combinatorial mutagenesis.

FIG. 11 shows pH-dependent binding of NA08 variants to immobilized CEA. The x-axis shows BLA activity, and the Y-axis shows CEA bound activity. Variant designations are shown in the top left corner.

FIG. 12 sets forth a CAB engineering summary. The left column refers to the protein designation. The middle column details cumulative changes from the previous line. The right column provides a putative reason for each of the mutations, as provided in the text of the document. For example, changes were made from CAB1 to CAB1.1 to increase the overall stability of the protein, as provided herein. As can be seen from the column, changes were also made to increase, among other things, the pH-dependent binding of a molecule, increase affinity and remove T-cell epitopes.

FIG. 13 sets forth binding of various CAB1 variants to immobilized CEA. Binding to CEA (x-axis) and BLA activity (y-axis) show, for example, different characteristics at different binding pHs.

FIG. 14 sets forth binding of various CAB1 variants to LS174T cells. Binding characteristics are shown for LS174T cells, the protocol as described herein. Again, different binding characteristics can be seen at different pHs.

FIG. 15 discloses relevant sequences as follows: FIG. 15A discloses the amino acid sequence of the SW149.5 protein; FIG. 15B discloses the amimo acid sequence of the CAB1.1 protein; FIG. 15C discloses the nucleotide sequence of the CAB1 gene; FIG. 15D discloses the amino acid sequence of the CAB1.2 protein; FIG. 15E discloses the amino acid sequence of the CAB1.4CDRs; FIG. 15F discloses the nucleotide sequence of the CAB1.4 CDRs; FIG. 15G discloses the nucleotide sequence of the entire CAB1.4 gene, including BLA, etc; FIG. 15H discloses the amino acid sequence of the CAB1.4 protein; FIG. 15I discloses the nucleotide sequence of the ZS CAB1.6 CDRs; FIG. 15J discloses the nucleotide sequence of the entire CAB1.6 gene, including BLA, etc.; FIG. 15K discloses the nucleotide sequence of the entire CAB1.6i gene, including BLA, etc.; FIG. 15L discloses the nucleotide sequence of the CAB1.7 CDRs; FIG. 15M discloses the nucleotide sequence of the entire CAB1.7 gene, including BLA, etc.; FIG. 15N discloses the nucleotide sequence of the entire CAB1.7i gene, including BLA, etc; FIG. 15O discloses the nucleotide sequence of the CAB1 CDRs; FIG. 15P discloses the nucleotide sequence for the entire CAB1.2 gene, including BLA, etc; FIG. 15Q discloses the amino acid sequence for the SW149.5 CDRs; FIG. 15R discloses the nucleotide sequence for the SW149.5 CDRs; FIG. 15S discloses the nucleotide sequence for the entire SW149.5, including BLA, etc.; FIG. 15T discloses the nucleotide sequence for BLA; FIG. 15U discloses the nucleotide sequence for CAB1.1.

FIG. 16 shows pharmacokinetics and tissue distribution of CAB1.11i and CAB 1.13i in T1918 tumorbearing athymic mice. The x-axis shows time in hours; the y-axis shows BLA activity:

FIG. 17 shows anti-tumor activity of C-Mel or glutaryl-C-Mel when administered 24 hrs after CAB 1.2 in LS174T SCID model as set forth in Example 10, where the x-axis is time in days, and the y-axis is tumor volume measured in mm³.

FIG. 18 shows toxicity-survival in anti-tumor activity of C-Mel and glutaryl-C-Mel when administered 24 hrs after CAB1.2 in LS174T SCID model as set forth in Example 10. The x-axis shows time in days, and the y-axis shows the number of living mice.

FIG. 19 shows toxicity-body weight of C-Mel and glutaryl-C-Mel and when administered 24 hrs after CAB 1.2 in LS174T SCID model as set forth in Example 10. The x-axis shows time in days, and the y-axis shows body weight percentage.

FIG. 20 shows animal weight effects after administration of CAB1.2/prodrug combinations compared with controls, as described in Example 12. The x-axis shows time in days, and the y-axis shows treatment group weight as measured in grams.

FIG. 21 plots survival of CAB1.2/prodrug combinations compared with controls. The x-axis shows time in days, and the y-axis shows the number of surviving animal.

FIG. 22 shows efficacy of the CAB1.2/prodrug combinations compared with controls, as shown in Example 12. The x-axis shows time in days, and the y-axis shows tumor volume measured in mm³. Groups are as follows: Group 1: CAB1.2/C-Mel (2.5 mg/kg, 18 hr); Group 2: CAB1.2/C-Mel (2.5 mg/kg, 36 hr); Group 3: CAB1.2/C-Mel (1 mg/kg, 24); Group 4: Untreated control; Group 5 CAB1.2 alone (2.5 mg/kg); Group 6 C-Mel alone; Group 7 Melphalan (10 mg/kg); Group 8 P97ADEPT/C-Mel (2.5 mg/kg, 18 hr); Group 9 BLA/C-Mel (1.5 mg/kg, 18 hr).

FIG. 23 shows efficacy, where the x-axis shows day number, and the y-axis shows tumor volume measured in mm3.

FIG. 24 discloses relevant sequences as follows: FIG. 24A discloses the amino acid sequence for CAB1.2i; FIG. 24B discloses the nucleotide sequence for CAB1.2i; FIG. 24C discloses the amino acid sequence for CAB1.13i and FIG. 24D discloses the nucleotide sequence for CAB1.13i.

FIGS. 25A and 25B set forth the amino acid and nucleotide sequence, respectively, for CAB1.11i.

FIG. 26 shows the results of IHC staining as set forth in Example 15. Column 1 shows Case ID; column 4 shows sample pathology; column 5 shows sample diagnosis; column 6 shows tissue of origin/site of finding; column 7 shows results of H&E staining, as set forth in Example; column 8 shows results of staining against the control, human cytokeratin; columns 9-12 show results of staining against relevant CAB; column 13 shows results of no antibody staining.

FIG. 27 shows the average tumor volume (27A) and average body weight (27B), as set forth in Example 16. The x-axis shows time, measured in days, and the y-axis shows tumor volume, measured in mm³, and percent body weight change, respectively.

FIG. 28 shows plasma concentration of GC-Mel at different time points. The x-axis shows time in minutes, and the y-axis shows concentration.

FIG. 29 shows and the exposure ratio with the tumor exposure to Mel. FIG. 29A shows the tumor/plasma CG-Mel exposure ratio, with the x-axis showing time, and the y-axis showing the tumor/plasma exposure ratio. FIG. 29B shows tumor exposure to Melphalan, the bars indicating time, and the y-axis showing normalized dose, as described in the Examples. FIG. 29C shows Melphain tumor plasma ratio after GC-Mel administration, as described herein, the bars showing time, and the y-axis showing the tumor/plasma exposure ratio.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are used as described below.

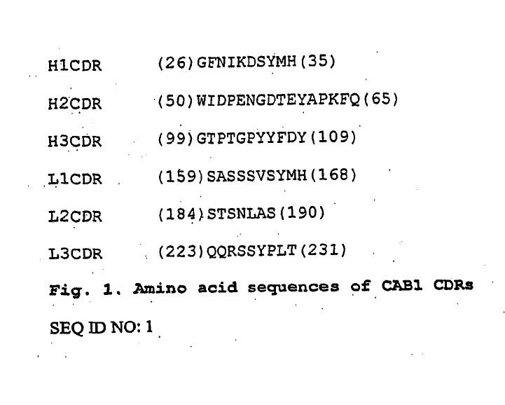

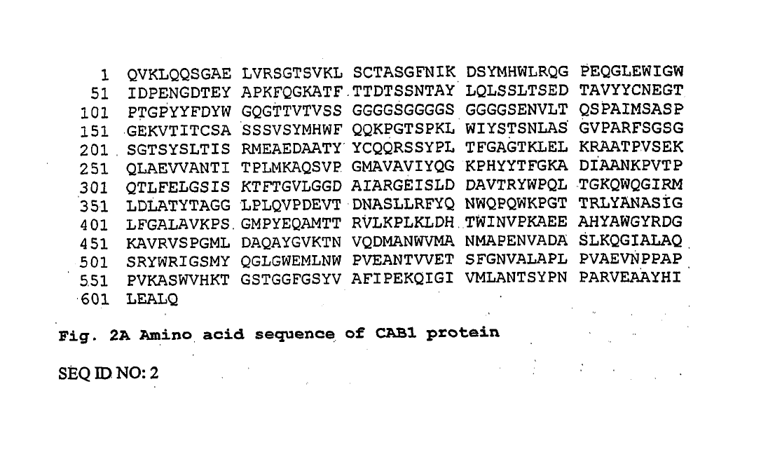

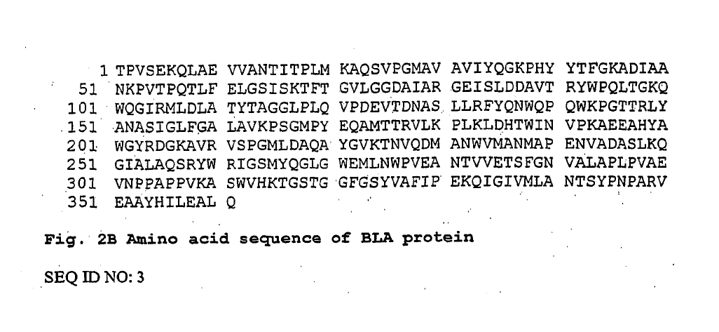

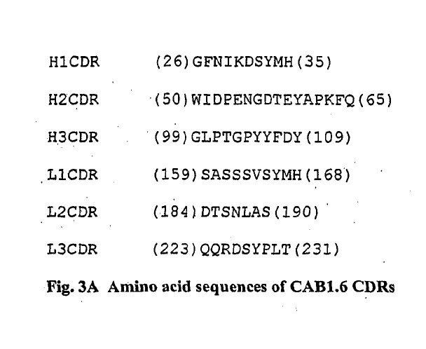

“CAB” molecule shall mean a targeted agent that binds to a CEA target or microtarget and has an unmodified or modified sequence and whose unmodified sequence comprises the amino acid sequence set forth in SEQ ID NO:1 or SEQ ID NO:2. SEQ ID NO:1 sets forth the amino acid sequence of the unmodified CDR portion of the CAB molecule of the present invention as shown in FIG. 1 SEQ ID NO:2 sets forth a CAB molecule that includes BLA as shown in FIG. 2 and position numbering shall be with respect to SEQ ID NO:1 and SEQ ID NO:2, as set forth in FIG. 1 and FIG. 2, respectively. CAB designations may be followed by a number to designate specific combinations of modifications of the present invention. For example, as set forth above, and throughout the rest of the application, CAB1.6 shall refer to a CAB molecule having the following modifications: T100L, S184D and S226D, wherein position numbering is with respect to SEQ ID NO:1; or a CAB molecule having the following mutations: K3Q, R13K, T16G, L37V, T100L, M146V, W181V, S184D and S226D, wherein position numbering is with respect to SEQ ID NO:2. Also, for example, CAB1.7i shall refer to a CAB molecule having the following modifications: K3Q, R13K, T16G, L37V, T100L, T102L, P104A, Y105I, F107N, M146V, S163A, S165Y, Y166A, W181V, S184D, S226D, K265A and S568A, wherein position numbering is with respect to SEQ ID NO:2 as shown in FIG. 2.

A “targeted agent” is a chemical entity that binds selectively to a microtarget of interest. Examples of targeted agents are antibodies, peptides and inhibitors. Of interest are targeted enzymes that have a desired catalytic activity and that can bind to one or more target structures with high affinity and selectivity. Targeted enzymes retain at least most of their activity while bound to a target.

A “binding moiety” is a part of a targeted agent (or an ADEPT costruct, e.g., CAB molecule) that binds a microtarget. A binding moiety can comprise more than one region, either contiguous or non-contiguous, of the CAB.

An “active moiety” is a part of a targeted agent (or an ADEPT construct, e.g., CAB molecule) that confers functionality to the agent. An active moiety can comprise more than one region, either contiguous or non-contiguous, of, for example, a CAB molecule. In particular, an active moiety can be a beta-lactamase.

The term “protein” is used interchangeably here with the terms “peptide” and “polypeptide,” and refers to a molecule comprising two or more amino acid residues joined by a peptide bond.

The terms “cell”, “cell line”, and “cell culture” can be used interchangeably and all such designations include progeny. The words “transformants” or “transformed cells” include the primary transformed cell and cultures derived from that cell without regard to number of transfers. All progeny may not precisely identical in DNA content, due to deliberate or inadvertent mutations. Mutant progeny that have the same functionality as screened for in the originally transformed cell are included in the a) definition of transformants. The cells can be prokaryotic or eukaryotic.

The term “oligonucleotide” as used herein is defined as a molecule comprised of two or more deoxyribonucleotides or ribonucleotides. The exact size will depend on many factors, which in turn depends on the ultimate function or use of the oligonucleotide. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods is provided in Goodchild, 1990, Bioconjugate Chemistry 1(3):165-187, incorporated herein by reference.

The term “primer” as used herein refers to an oligonucleotide capable of acting as a point of initiation of synthesis when placed under conditions in which primer extension is initiated. Synthesis of a primer extension product that is complementary to a nucleic acid strand is initiated in the presence of the requisite four different nucleoside triphosphates and a DNA polymerase in an appropriate buffer at a suitable temperature. A “buffer” includes a buffer, cofactors (such as divalent metal ions) and salt (to provide the appropriate ionic strength), adjusted to the desired pH.

A primer that hybridizes to the non-coding strand of a gene sequence (equivalently, is a subsequence of the noncoding strand) is referred to herein as an “upstream” or “forward” primer. A primer that hybridizes to the coding strand of a gene sequence is referred to herein as an “downstream” or “reverse” primer.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine), nonpolar side chains (e.g., alanine, valine, leucine, isoleucine, proline, phenylalanine, methionine, tryptophan, cysteine, glycine), beta-branched side chains (e.g., threonine, valine, isoleucine) and aromatic side chains (e.g., tyrosine, phenylalanine, tryptophan, histidine). Standard three-letter or one-letter amino acid abbreviations are used herein. Equivalent substitutions may be included within the scope of the claims.

The peptides, polypeptides and proteins of the invention can comprise one or more non-classical amino acids. Non-classical amino acids include but are not limited to the D-isomers of the common amino acids, α-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

The term “Ab” or “antibody” refers to polyclonal and monoclonal antibodies, chimeric antibodies, humanized antibodies, human antibodies, immunoglobulins or antibody or functional fragments of an antibody that binds to a target antigen. Examples of such functional entities include complete antibody molecules, antibody fragments, such as Fv, single chain Fv, complementarity determining regions (CDRs), V_{L} (light chain variable region), V_{H} (heavy chain variable region) and any combination of those or any other functional portion of an immunoglobulin peptide capable of binding to target antigen. In the examples, the construct has the following order: vL-(GGGGS)₆-vH; however, the example is non-limiting, and all orders of vL and vH, are contemplated to be within the scope of the invention.

The term “prodrug” refers to a compound that is converted via one or more enzymatically catalyzed steps into an active compound that has an increased pharmacological activity relative to the prodrug. A prodrug can comprise a pro-part or inactive moiety and a drug or active drug or detectable moiety. Optionally, the prodrug also contains a linker. For example, the prodrug can be cleaved by an enzyme to release an active drug. Alternatively, an enzyme could alter the prodrug to release a detectable moiety. In a more specific example, prodrug cleavage by the targeted enzyme releases the active drug into the vicinity of the target bound to the targeted enzyme. “Pro-part” and “inactive moiety” refer to the inactive portion of the prodrug after it has been converted. For example, if a prodrug comprises a PEG molecule linked by a peptide to an active drug, the pro-part is the PEG moiety with or without a portion of the peptide linker.

As used herein, “GC-Mel” shall refer to the prodrug glutaryl-cephalosporin-melphalan as disclosed, for example, in Senter et al., U.S. Pat. No. 5,773,435, which is incorporated by reference herein, including any drawings.

The term “drug” and “active drug” and “detectable moiety” refer to the active moieties of a prodrug. After cleavage of the prodrug by a targeted enzyme, the active drug acts therapeutically upon the targeted tumor, cell, infectious agent or other agent of disease. The detectable moiety acts as a diagnostic tool, and such detectable moieties are intended to be within the scope of the claims. The active drug can be any chemical entity that is able to kill a cell or inhibit cell proliferation.

As used herein, “Mel” shall mean Melphalan. The structure of Mel is well known in the art and can also be found in U.S. Pat. No. 5,773,435.

The term “% sequence homology” is used interchangeably herein with the terms “% homology,” “% sequence identity” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly, a homologue of a given sequence has greater than 80% sequence identity over a length of the given sequence. Exemplary levels of sequence identity include, but are not limited to, 60, 70, 80, 85, 90, 95, 98 or 99% or more sequence identity to a given sequence.

Exemplary computer programs that can be used to determine identity between two sequences include, but are not limited to, the suite of BLAST programs, e.g., BLASTN, BLASTX, and TBLASTX, BLASTP and TBLASTN, which are well-known to one skilled and the art. See also Altschul et al., 1990, J. Mol. Biol. 215: 403-10 and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Altschul, et al., 1997.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

In a first aspect, the invention is drawn to a CAB molecule comprising a modified amino acid sequence. In one embodiment, the CAB molecule has the unmodified sequence set forth in SEQ ID NO:1. In one embodiment, the CAB molecule has an amino acid sequence modified from the amino acid sequence set forth in SEQ ID NO:1, and the modification is at least one position selected from the group consisting of positions 100, 102, 104, 105, 107, 163, 165, 166, 184 and 226, wherein position numbering is with respect to SEQ ID NO:1 as shown in FIG. 1. In a preferred embodiment, the CAB molecule comprises modifications at positions 100, 184 and 226.

In a preferred embodiment, the CAB molecule comprises modifications at positions 100, 102, 104, 105, 107, 163, 165, 166, 184 and 226. In a preferred embodiment, the CAB molecule comprises modifications at positions 100, 102, 104, 105, 107, 163, 165, 166 and 226.

In a preferred embodiment, the modification is at least one selected from the group consisting of T100L, T102L, P104A, Y105I, F107N, S163A, S165Y, Y166A, S184D and S226D, wherein position numbering is with respect to SEQ ID NO:1 as shown in FIG. 1. In a preferred embodiment, the CAB molecule comprises a CAB1.6 molecule, the CAB1.6 molecule having the following modifications: T100L, S184D and S226D. In a preferred embodiment, the CAB molecule comprises a CAB1.7 molecule, the CAB1.7 molecule having the following modifications: T100L, T102L, P104A, Y105I, F107N, S163A, S165Y, Y166A, S184D and S226D. In a preferred embodiment, the CAB molecule comprises a CAB1.13 molecule, the CAB1.13 molecule having the following modifications: T100L, T102L, P104A, Y105I, F107N, S163A, S165Y, Y166A and S226D.

In a preferred embodiment, the CAB molecule comprises the scFV portion of CAB1.2 (SEQ ID NO:1), CAB1.6 (SEQ ID NO:5), CAB1.7 (SEQ ID NO:6) or CAB1.13 as set forth in FIG. 25.

In a preferred embodiment, the CAB molecule further comprises a beta-lactamase molecule. In a preferred embodiment, the CAB molecule has an amino acid sequence that is unmodified or modified from the amino acid sequence set forth in SEQ ID NO:2, and the modification is at least one position selected from the group consisting of positions: 3, 13, 16, 37, 100, 102, 104, 105, 107, 146, 163, 165, 166, 181, 184, 226, 265 and 568, wherein position numbering is with respect to SEQ ID NO:2 as shown in FIG. 2. In a preferred embodiment, the modifications are at positions 3, 13, 16, 37, 100, 146, 181, 184 and 226. In a preferred embodiment, the modifications are at positions 3, 13, 16, 37, 100, 102, 104, 105, 107, 146, 163, 165, 166, 181, 184 and 226. In a preferred embodiment, the modifications are at positions 265 and 568. In a preferred embodiment, the modifications are at positions 3, 13, 16, 37, 100, 102, 104, 105, 107, 146, 163, 165, 166, 181, 184, 226, 268 and 568. In a preferred emobodiment, the modifications are at 3, 13, 16, 37, 100, 102, 104, 105, 107, 146, 163, 165, 166, 181, 226, 265 and 568.

In a preferred embodiment, the CAB molecule further comprises a beta-lactamase molecule. In a preferred embodiment, the CAB molecule has an amino acid sequence modified from the amino acid sequence set forth in SEQ ID NO:2, and the modification is at least one position selected from the group consisting of positions: 3, 13, 16, 37, 100, 102, 104, 105, 107, 146, 163, 165, 166, 181, 184, 226, 265 and 568, wherein position numbering is with respect to SEQ ID NO:2 as shown in FIG. 2. In a preferred embodiment, the modifications are at positions 3, 13, 16, 37, 100, 146, 181, 184 and 226.

In a preferred embodiment, the modifications are at positions 3, 13, 16, 37, 100, 102, 104, 105, 107, 146, 163, 165, 166, 181, 184 and 226. In a preferred embodiment, the modifications are at positions 3, 13, 16, 37, 100, 102, 104, 105, 107, 146, 163, 165, 166, 181, 184, 226, 265 and 568.

In a preferred embodiment, the CAB molecule has modifications comprising at least one modification selected from the group consisting of K3Q, R13K, T16G, L37V, T100L, T102L, P104A, Y105I, F107N, M146V, S163A, S165Y, Y166A, W181V, S184D, S226D, K265A and S568A, wherein position numbering is with respect to SEQ ID NO:2 as shown in FIG. 2. In a preferred embodiment, the CAB molecule comprises a CAB1.2i molecule, the CAB1.2i molecule comprising the following modifications: K265A and S568A. In a preferred embodiment, the CAB molecule comprises a CAB1.6 molecule, the CAB1.6 molecule comprising the following modifications: K3Q, R13K, T16G, L37V, T100L, M146V, W181V, S184D and S226D. In a preferred embodiment, the CAB molecule comprises a CAB1.6i molecule, the CAB1.6i molecule comprising the following modifications: K3Q, R13K, T16G, L37V, T100L, M146V, W181V, S184D, S226D, K265A and S568A. In a preferred embodiment, the CAB molecule comprises a CAB1.7 molecule, the CAB1 molecule comprising the following modifications: K3Q, R13K, T16G, L37V, T100L, T102L, P104A, Y105I, F107N, M146V, S163A, S165Y, Y166A, W181V, S184D and S226D. In a preferred embodiment, the CAB comprises a CAB1.7i molecule, the CAB1.7i molecule comprising the following modifications: K3Q, R13K, T16G, L37V, T100L, T102L, P104A, Y105I, F107N, M146V, S163A, S165Y, Y166A, W181V, S184D, S226D, K265A and S568A. In a preferred embodiment, the CAB molecule comprises a CAB1.13 molecule, the CAB1.13 molecule comprising the following modifications: K3Q, R13K, T16G, L37V, T100L, T102L, P104A, Y105I, F107N, M146V, S163A, S165Y, Y166A, W181V and S226D. In a preferred embodiment, the CAB comprises a CAB1.13i molecule, the CAB1.13i molecule comprising the following modifications: K3Q, R13K, T16G, L37V, T100L, T102L, P104A, Y105I, F107N, M146V, S163A, S165Y, Y166A, W181V, S226D, K265A and S568A.

In a preferred embodiment, the CAB molecule comprises CAB1.2 (SEQ ID NO:2) or CAB1.2i as set forth in FIG. 25, CAB1.6 (SEQ ID NO:7), CAB1.6i (SEQ ID NO:8), CAB1.7 (SEQ ID NO:9), CAB1.7i (SEQ ID NO:10), CAB1.13 as set forth in FIG. 25 or CAB1.13i as set forth in FIG. 25.

In another embodiment, the CAB is an MDTA as described in PCT Application Number US03/18200, filed Jun. 12, 2002 and incorporated herein by reference in its entirety. Some of the CAB molecules of the present invention have been shown to preferentially bind to a microtarget present on a target relative to binding of a non-target. The difference in binding can be caused by any difference between the target and non-target such as, for example, a difference in pH, oxygen pressure, concentration of solutes or analytes (e.g., lactic acid, sugars or other organic or inorganic molecules), temperature, light or ionic strength. Preferential binding of the CABs of the current invention can be used to bind to a microtarget under a desired set of conditions, identify a target in vitro, ex vivo, in situ or in vivo (e.g., a target tissue in a subject), kill a target cell or tissue, convert a prodrug into an active drug in or near a target tissue. It also can be used as surface catalysts, for example, a targeted laccase. Other uses include, e.g., targeted generation of a compound (e.g., H₂O₂ from glucose) and the targeted destruction of compounds (e.g., a metabolite or signalling molecule from a particular tissue).

In one embodiment, the CAB is selected, made or modified using an affinity maturation method, e.g., as described in PCT application, filed Juine 12, 2002 and incorporated herein by reference in its entirety.

In another embodiment, the CAB is selected, made or modified using a loop-grafting method, e.g., as described in U.S. patent application Ser. No. 10/170,387, filed Jun. 12, 2002 and incorporated herein by reference in its entirety.

In another embodiment, the CAB is a multifunctional polypeptide, e.g., as described in U.S. patent application Ser. No. 10/170,729, filed Jun. 12, 2002 and incorporated herein by reference in its entirety.

In another embodiment, the CABs of the invention are used for diagnostic or therapeutic application such as those disclosed, for example, in U.S. Pat. No. 4,975,278, which is incorporated herein by reference in its entirety, as well as methods well-known in the art.

In one embodiment, the CAB molecule further comprises an active moiety. The active moiety can be a molecule, or a part of a molecule, that has an activity. The activity can be any activity. Examples of types of activities that the active moiety can have include, for example, a detectable activity, an enzymatic activity, a therapeutic activity, a diagnostic activity, a toxic activity or a binding activity. The active moiety can be a discrete part of the CAB, for example, an enzyme that is fused or conjugated to the binding moiety, or it can be an integral part of the CAB, for example, binding of the CAB to the microtarget can activate or inhibit an activity of the microtarget or the target, or the CAB can be a targeted enzyme of the type discussed below and in copending U.S. patent application Ser. Nos. 10/022,073 and 10/022,097, incorporated herein by reference in their entireties.

In another embodiment, the active moiety exhibits enzymatic activity, e.g., it is an enzyme or an active fragment or derivative of an enzyme. Of particular interest are enzymes that can be used to activate a prodrug in a therapeutic setting. A large number of enzymes with different catalytic modes of action have been used to activate prodrugs. See, e.g., Melton & Knox Enzyme-prodrug strategies for cancer therapy (1999) and Bagshawe et al., Curr Opin Immunol 11:579 (1999). Examples of types of enzymes that can be used to make the CABs of the present invention include, but are not limited to, proteases, carboxypeptidases, β-lactamases, asparaginases, oxidases, hydrolases, lyases, lipases, cellulases, amylases, aldolases, phosphatases, kinases, tranferases, polymerases, nucleases, nucleotidases, laccases, reductases, and the like. See, e.g., co-pending U.S. patent application Ser. No. 09/954,385, filed Sep. 12, 2001, incorporated herein by reference in its entirety. As such, CABs of the invention can, for example, exhibit protease, carboxypeptidase, β-lactamase, asparaginase, oxidase, hydrolase, lyase, lipase, cellulase, amylase, aldolase, phospatase, kinase, tranferase, polymerase, nuclease, nucleotidase, laccase or reductase activity or the like. Examples of enzymes that can be used are those that can activate a prodrug, discussed below, and those that can produce a toxic agent from a metabolite, e.g., hydrogen peroxide from glucose. See Christofidou-Solomidou et al, 2000, Am J Physiol Lung Cell Mol Physiol 278:L794.

In one embodiment, the present invention provides a CAB further comprising a β-lactamase (“BLA”). In another embodiment, the BLA is a targeted enzyme as described in co-pending U.S. patent application Ser. Nos. 10/022,073 and 10/022,097, incorporated herein by reference in their entirety.

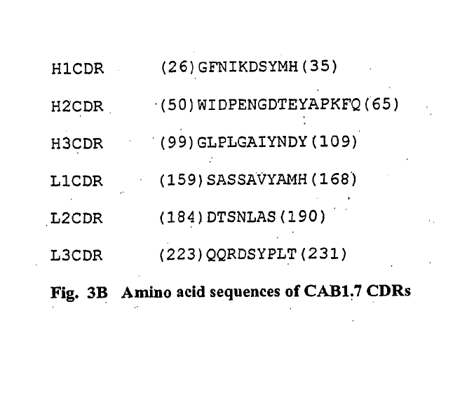

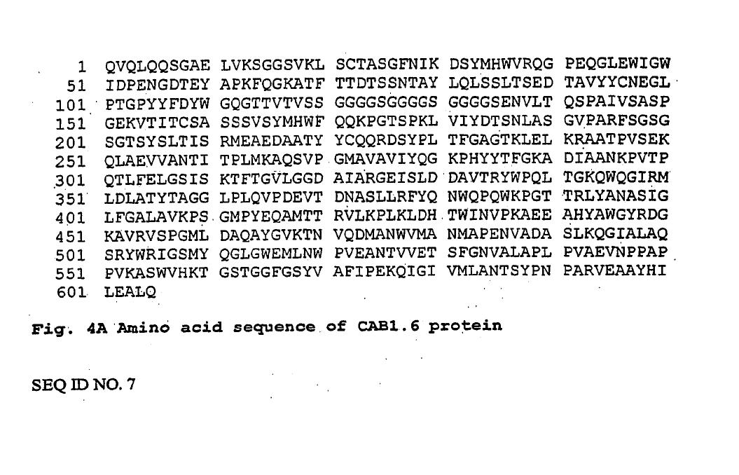

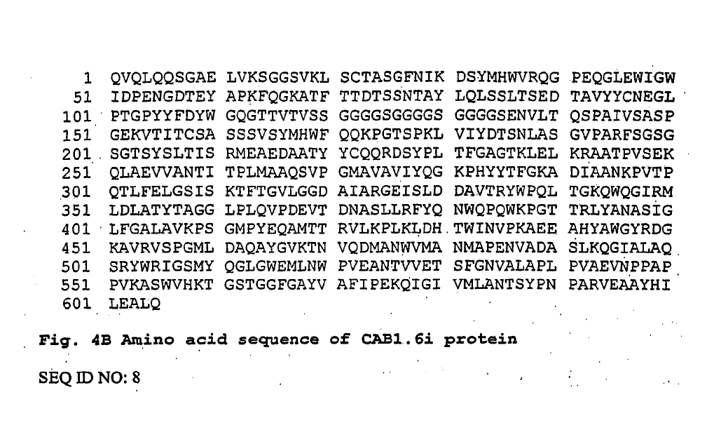

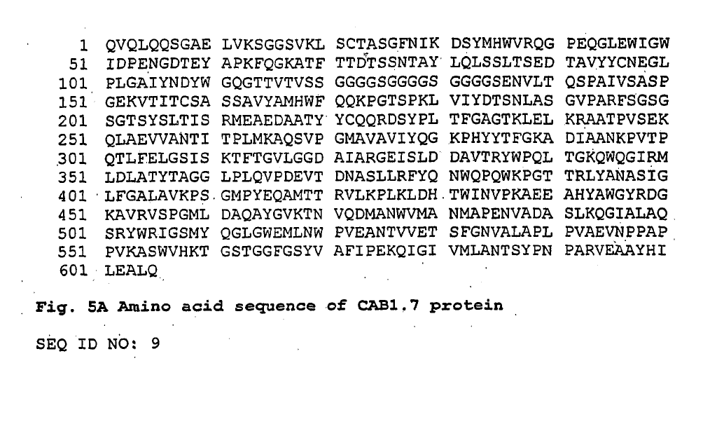

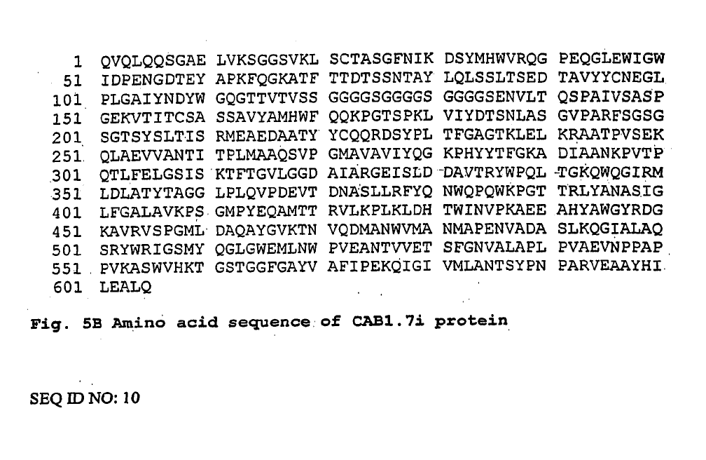

BLA enzymes are widely distributed in both gram-negative and gram-positive bacteria. BLA sequences are well known. A representative example of a BLA sequence is depicted in FIG. 3. BLA enzymes vary in specificity, but have in common that they hydrolyze β-lactams, producing substituted β-amino acids. Thus, they confer resistance to antibiotics containing β-lactams. Because BLA enzymes are not endogenous to mammals, they are subject to minimal interference from inhibitors, enzyme substrates, or endogenous enzyme systems (unlike proteases), and therefore are particularly well-suited for therapeutic administration. BLA enzymes are further well-suited to the therapeutic methods of the present invention because of their small size (BLA from E. cloacae is a monomer of 39 kD; BLA from E. coli is a monomer of 30 kD) and because they have a high specific activity against their substrates and have optimal activity at 37° C. See Melton et al., Enzyme-Prodrug Strategies for Cancer Therapy, Kluwer Academic/Plenum Publishers, New York (1999).

Examples of specific BLAs that can be used to make the CABs of the present invention include, but are not limited to, Class A, B, C or D β-lactamase, β-galactosidase, see Benito et al., FEMS Microbiol. Lett. 123:107 (1994), fibronectin, glucose oxidase, glutathione S-transferase, see Napolitano et al., Chem. Biol. 3:359 (1996) and tissue plasminogen activator, see Smith et al., J. Biol. Chem. 270:30486 (1995). The β-lactamases have been divided into four classes based on their sequences. See Thomson et al., 2000, Microbes and Infection 2:1225-35. The serine β-lactamases are subdivided into three classes: A (penicillinases), C (cephalosporinases) and D (oxacillnases). Class B β-lactamases are the zinc-containing or metallo β-lactamases. Any class of BLA can be utilized to generate an CAB of the invention.

In one embodiment of the invention, the BLA has a specific activity greater than about 0.01 U/pmol against nitrocefin using the assay described in U.S. patent application Ser. No. 10/022,097. In another embodiment, the specific activity is greater than about 0.1 U/pmol. In another embodiment, the specific activity is greater than about 1 U/pmol. Preferably, these specific activities refer to the specific activity of the BLA when it is bound to a microtarget.

In one embodiment, the BLA enzyme in the CAB comprises the amino acid sequence set forth in SEQ ID NO:3. In another embodiment, the BLA enzyme in the CAB is at least 50%, 60%, 70%, 80%, 90%, 95%, 98% or 99% or more identical to the sequence depicted in FIG. 2.

In a preferred embodiment, the CAB is CAB1.6, CAB1.6i, CAB1.7 or CAB1.7i.

The targets bound by the CAB, or one or more binding moieties, can be any substance or composition to which a molecule can be made to bind to CEA. In one embodiment, the target is a surface. In one embodiment, the surface is a biological surface. In another embodiment, the biological surface is a surface of an organ. In another embodiment, the biological surface is a surface of a tissue. In another embodiment, the biological surface is a surface of a cell. In another embodiment, the biological surface is a surface of a diseased organ, tissue or cell. In another embodiment, the biological surface is a surface of a normal or healthy organ, tissue or cell. In another embodiment, the surface is a macromolecule in the interstitial space of a tissue. In another embodiment, the biological surface is the surface of a virus or pathogen. In another embodiment, the surface is a non-biological surface. In another embodiment, the non-biological surface is a surface of a medical device. In another embodiment, the medical device is a therapeutic device. In another embodiment, the therapeutic device is an implanted therapeutic device. In another embodiment, the medical device is a diagnostic device. In another embodiment, the diagnostic device is a well or tray.

Sources of cells or tissues include human, all other animals, bacteria, fungi, viruses and plant. Tissues are complex targets and refer to a single cell type, a collection of cell types or an aggregate of cells generally of a particular kind. Tissue may be intact or modified. General classes of tissue in humans include but are not limited to epithelial tissue, connective tissue, nerve tissue and muscle tissue.

In another embodiment, the target is a cancer-related target that expresses CEA or that has CEA bound to itself or that has CEA located in its vicinity. The cancer-related target can be any target that a composition of the invention binds to as part of the diagnosis, detection or treatment of a cancer or cancer-associated condition in a subject, for example, a cancerous cell, tissue or organ, a molecule associated with a cancerous cell, tissue or organ, or a molecule, cell, tissue or organ that is associated with a cancerous cell, tissue or organ (e.g., a tumor-bound diagnostic or therapeutic molecule administered to a subject or to a biopsy taken from a subject, or a healthy tissue, such as vasculature, that is associated with cancerous tissue).

In a second aspect, the invention is drawn to a nucleic acid encoding a CAB molecule as set forth herein. The nucleic acid can be, for example, a DNA or an RNA. The present invention also provides a plasmid comprising a nucleic acid encoding a polypeptide comprising all or part of a CAB. The plasmid can be, for example, an expression plasmid that allows expression of the polypeptide in a host cell or organism, or in vitro. The expression vector can allow expression of the polypeptide in, for example, a bacterial cell. The bacterial cell can be, for example, an E. coli cell.

Because of the redundancy in the genetic code, typically a large number of DNA sequences encode any given amino acid sequence and are, in this sense, equivalent. As described below, it may be desirable to select one or another equivalent DNA sequences for use in a expression vector, based on the preferred codon usage of the host cell into which the expression vector will be inserted. The present invention is intended to encompass all DNA sequences that encode the desired CAB.

An operable expression clone may be used and is constructed by placing the coding sequence in operable linkage with a suitable control sequence in an expression vector. The vector can be designed to replicate autonomously in the host cell or to integrate into the chromosomal DNA of the host cell. The resulting clone is used to transform a suitable host, and the transformed host is cultured under conditions suitable so for expression of the coding sequence. The expressed CAB is then isolated from the medium or from the cells, although recovery and purification of the CAB may not be necessary in some instances.

Construction of suitable clones containing the coding sequence and a suitable control sequence employ standard ligation and restriction techniques that are well understood in the art. In general, isolated plasmids, DNA sequences or synthesized oligonucleotides are cleaved, modified and religated in the form desired. Suitable restriction sites can, if not normally available, be added to the ends of the coding sequence so as to facilitate construction of an expression clone.

Site-specific DNA cleavage is performed by treating with a suitable restriction enzyme (or enzymes) under conditions that are generally understood in the art and specified by the manufacturers of commercially available restriction enzymes. See, e.g., product catalogs from Amersham (Arlington Heights, Ill.), Roche Molecular Biochemicals (Indianapolis, Ind.), and New England Biolabs (Beverly, Mass.). Incubation times of about one to two hours at a temperature that is optimal for the particular enzyme are typical. After each incubation, protein is removed by extraction with phenol and chloroform; this extraction can be followed by ether extraction and recovery of the DNA from aqueous fractions by precipitation with ethanol. If desired, size separation of the cleaved fragments may be performed by polyacrylamide gel or agarose gel electrophoresis using standard techniques. See, e.g., Maxam et al., 1980, Methods in Enzymology 65:499-560.

Ligations can be performed, for example, in 15-30 μl volumes under the following standard conditions and temperatures: 20 mM Tris-Cl, pH 75, 10 mM MgCl₂, 10 mM DTT, 33 μg/ml BSA, 10-50 mM NaCl, and either 40 μM ATP and 0.01-0.02 (Weiss) units T4 DNA ligase at 0° C. (for ligation of fragments with complementary single-stranded ends) or 1 mM ATP and 0.3-0.6 units T4 DNA ligase at 14° C. (for “blunt end” ligation). Intermolecular ligations of fragments with complementary ends are usually performed at 33-100 μg/ml total DNA concentrations (5-100 nM total ends concentration). Intermolecular blunt end ligations (usually employing a 20-30 fold molar excess of linkers, optionally) are performed at 1 μM total ends concentration.

Correct ligations for plasmid construction can be confirmed using any suitable method known in the art. For example, correct ligations for plasmid construction can be confirmed by first transforming a suitable host, such as E. coli strain DG101 (ATCC 47043) or E. coli strain DG116 (ATCC 53606), with the ligation mixture. Successful transformants are selected by ampicillin, tetracycline or other antibiotic resistance or sensitivity or by using other markers, depending on the mode of plasmid construction, as is understood in the art. Plasmids from the transformants are then prepared according to the method of Clewell et al., 1969, Proc. Natl. Acad. Sci. USA 62:1159, optionally following chloramphenicol amplification. See Clewell, 1972, J. Bacteriol. 110:667. Alternatively, plasmid DNA can be prepared using the “Base-Acid” extraction method at page 11 of the Bethesda Research Laboratories publication Focus 5 (2), and very pure plasmid DNA can be obtained by replacing steps 12 through 17 of the protocol with CsCl/ethidium bromide ultracentrifugation of the DNA. As another alternative, a commercially available plasmid DNA isolation kit, e.g., HISPEEDT™, QIAFILTER™ and QIAGEN® plasmid DNA isolation kits (Qiagen, Valencia Calif.) can be employed following the protocols supplied by the vendor. The isolated DNA can be analyzed by, for example, restriction enzyme digestion and/or sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499.

The control sequences, expression vectors and transformation methods are dependent on the type of host cell used to express the gene. Generally, procaryotic, yeast, insect or mammalian cells are used as hosts. Procaryotic hosts are in general the most efficient and convenient for the production of recombinant proteins and are therefore preferred for the expression of the protein.

The procaryote most frequently used to express recombinant proteins is E. coli. However, microbial strains other than E. coli can also be used, such as bacilli, for example Bacillus subtilis, various species of Pseudomonas and Salmonella, and other bacterial strains. In such procaryotic systems, plasmid vectors that contain replication sites and control sequences derived from the host or a species compatible with the host are typically used.

For expression of constructions under control of most bacterial promoters, E. coli K12 strain MM294, obtained from the E. coli Genetic Stock Center under GCSC #6135, can be used as the host. For expression vectors with the P_{L}N_{RBS} or P_{L} T7_{RBS} control sequence, E. coli K12 strain MC1000 lambda lysogen, N₇N₅₃cI857 SusP₈₀, ATCC 39531 may be used. E. coli DG116, which was deposited with the ATCC (ATCC 53606) on Apr. 7, 1987, and E. coli KB2, which was deposited with the ATCC (ATCC 53075) on Mar. 29, 1985, are also useful host cells. For M13 phage recombinants, E. coli strains susceptible to phage infection, such as E. coli K12 strain DG98 (ATCC 39768), are employed. The DG98 strain was deposited with the ATCC on Jul. 13, 1984.

For example, E. coli is typically transformed using derivatives of pBR322, described by Bolivar et al., 1977, Gene 2:95. Plasmid pBR322 contains genes for ampicillin and tetracycline resistance. These drug resistance markers can be either retained or destroyed in constructing the desired vector and so help to detect the presence of a desired recombinant. Commonly used procaryotic control sequences, i.e., a promoter for transcription initiation, optionally with an operator, along with a ribosome binding site sequence, include the β-lactamase (penicillinase) and lactose (lac) promoter systems, see Chang et al., 1977, Nature 198:1056, the tryptophan (tip) promoter system, see Goeddel et al., 1980, Nuc. Acids Res. 8:4057, and the lambda-derived P_{L} promoter, see Shimatake et al., 1981, Nature 292:128, and gene N ribosome binding site (N_{RBS}). A portable control system cassette is set forth in U.S. Pat. No. 4,711,845, issued Dec. 8, 1987. This cassette comprises a P_{L} promoter operably linked to the N_{RBS} in turn positioned upstream of a third DNA sequence having at least one restriction site that permits cleavage within six base pairs 3′ of the N_{RBS} sequence. Also useful is the phosphatase A (phoA) system described by Chang et al., in European Patent Publication No. 196,864, published Oct. 8, 1986. However, any available promoter system compatible with procaryotes can be used to construct a expression vector of the invention.

In addition to bacteria, eucaryotic microbes, such as yeast, can also be used as recombinant host cells. Laboratory strains of Saccharomyces cerevisiae, Baker's yeast, are most often used, although a number of other strains are commonly available. While vectors employing the two micron origin of replication are common, see Broach, 1983, Meth. Enz. 101:307, other plasmid vectors suitable for yeast expression are known. See, e.g., Stinchcomb et al., 1979, Nature 282:39; Tschempe et al., 1980, Gene 10:157; and Clarke et al., 1983, Meth. Enz. 101:300. Control sequences for yeast vectors include promoters for the synthesis of glycolytic enzymes. See Hess et al., 1968, J. Adv. Enzyme Reg. 7:1.49; Holland et al., 1978, Biotechnology 17:4900; and Holland et al., 1981, J. Biol. Chem. 256:1385. Additional promoters known in the art include the promoter for 3-phosphoglycerate kinase, see Hitzeman et al., 1980, J. Biol. Chem. 255:2073, and those for other glycolytic enzymes, such as glyceraldehyde 3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase and glucokinase. Other promoters that have the additional advantage of transcription controlled by growth conditions are the promoter regions for alcohol dehydrogenase 2, isocytochrome C, acid phosphatase, degradative enzymes associated with nitrogen metabolism and enzymes responsible for maltose and galactose utilization.

Terminator sequences may also be used to enhance expression when placed at the 3′ end of the coding sequence. Such terminators are found in the 3′ untranslated region following the coding sequences in yeast-derived genes. Any vector containing a yeast-compatible promoter, origin of replication and other control sequences is suitable for use in constructing yeast expression vectors.

The coding sequence can also be expressed in eucaryotic host cell cultures derived from multicellular organisms. See, e.g., Tissue Culture, Academic Press, Cruz and Patterson, editors (1973). Useful host cell lines include COS-7, COS-A2, CV-1, murine cells such as murine myelomas N51 and VERO, HeLa cells and Chinese hamster ovary (CHO) cells. Expression vectors for such cells ordinarily include promoters and control sequences compatible with mammalian cells such as, for example, the commonly used early and late promoters from Simian Virus 40 (SV 40), see Fiers et al., 1978, Nature 273:113, or other viral promoters such as those derived from polyoma, adenovirus 2, bovine papilloma virus (BPV) or avian sarcoma viruses, or immunoglobulin promoters and heat shock promoters.

Enhancer regions are also important in optimizing expression; these are, generally, sequences found upstream of the promoter region. Origins of replication may be obtained, if needed; from viral sources. However, integration into the chromosome is a common mechanism for DNA replication in eucaryotes.

Plant cells can also be used as hosts, and control sequences compatible with plant cells, such as the nopaline synthase promoter and polyadenylation signal sequences, see Depicker et al., 1982, J. Mol. Appl. Gen. 1:561, are available. Expression systems employing insect cells utilizing the control systems provided by baculovirus vectors have also been described. See Miller et al., in Genetic Engineering (1986), Setlow et al., eds., Plenum Publishing, Vol. 8, pp. 277-97. Insect cell-based expression can be accomplished in Spodoptera frugipeida. These systems are also successful in producing recombinant enzymes.

Depending on the host cell used, transformation is done using standard techniques appropriate to such cells. The calcium treatment employing calcium chloride, as described by Cohen, 1972, Proc. Natl. Acad. Sci. USA 69:2110, is used for procaryotes or other cells that contain substantial cell wall barriers. Infection with Agrobacterium tumefaciens, see Shaw et al., 1983, Gene 23:315, is used for certain plant cells. For mammalian cells, the calcium phosphate precipitation method of Graham et al., 1978, Virology 52:546 is preferred. Transformations into yeast are carried out according to the method of Van Solingen et al., 1977, J. Bact. 130:946, and Hsiao et al., 1979, Proc. Natl. Acad. Sci. USA 76:3829.

It may be desirable to modify the sequence of a DNA encoding a polypeptide comprising all or part of a CAB of the invention to provide, for example, a sequence more compatible with the codon usage of the host cell without modifying the amino acid sequence of the encoded protein. Such modifications to the initial 5-6 codons may improve expression efficiency. DNA sequences which have been modified to improve expression efficiency, but which encode the same amino acid sequence, are considered to be equivalent and encompassed by the present invention.

A variety of site-specific primer-directed mutagenesis methods are available and well-known in the art See, e.g., Sambrook et al., Molecular Cloning: A Laboratory Manual, Cold Spring Harbor, 1989, second edition, chapter 15.51, “Oligonucleotide-mediated mutagenesis,” which is incorporated herein by reference. The polymerase chain reaction (PCR) can be used to perform site-specific mutagenesis. In another technique now standard in the art, a synthetic oligonucleotide encoding the desired mutation is used as a primer to direct synthesis of a complementary nucleic acid sequence contained in a single-stranded vector, such as pBSM13+ derivatives, that serves as a template for construction of the extension product of the mutagenizing primer. The mutagenized DNA is transformed into a host bacterium, and cultures of the transformed bacteria are plated and identified. The identification of modified vectors may involve transfer of the DNA of selected transformants to a nitrocellulose filter or other membrane and the “lifts” hybridized with kinased synthetic mutagenic primer at a temperature that permits hybridization of an exact match to the modified sequence but prevents hybridization with the original unmutagenized strand. Transformants that contain DNA that hybridizes with the probe are then cultured (the sequence of the DNA is generally confirmed by sequence analysis) and serve as a reservoir of the modified DNA.

Once the polypeptide has been expressed in a recombinant host cell, purification of the polypeptide may be desired. A variety of purification procedures can be used.

In another embodiment, a nucleic acid encoding the CAB hybridizes to a nucleic acid complementary to a nucleic acid encoding any of the amino acid sequences disclosed herein under highly stringent conditions. The highly stringent conditions can be, for example, hybridization to filter-bound DNA in 0.5 M NaHPO₄, 7% sodium dodecyl sulfate (SDS), 1 mM EDTA at 65° C. and washing in 0.1×SSC/0.1% SDS at 68° C. (Ausubel et al., eds., 1989, Current Protocols in Molecular Biology, Vol. I, Green Publishing Associates, Inc., and John Wiley & Sons, Inc., New York, at p. 2.10.3). Other highly stringent conditions can be found in, for example, Current Protocols in Molecular Biology, at pages 2.10.1-16 and Molecular Cloning: A Laboratory Manual, 2d ed., Sambrook et al. (eds.), Cold Spring Harbor Laboratory Press, 1989, pages 9.47-57. In another embodiment, moderately stringent conditions are used. The moderately stringent conditions can be, for example, washing in 0.2×SSC/0.1% SDS at 42° C. (Ausubel et al., 1989, supra). Other moderately stringent conditions can be found in, for example, Current Protocols in Molecular Biology, Vol. I, Ausubel et al. (eds.), Green Publishing Associates, Inc., and John Wiley & Sons, Inc., 1989, pages 2.10.1-16 and Molecular Cloning: A Laboratory Manual, 2d ed., Sambrook et al. (eds.), Cold Spring Harbor Laboratory Press, 1989, pages 9:47-57.

In a third aspect the present invention provides a method of treating a subject in need thereof comprising administering to a subject a CAB and a prodrug that is a substrate of the CAB. In another embodiment, the invention provides a method of treating a subject by administering to the subject a CAB, further comprising a BLA, and a prodrug that is converted by the BLA into an active drug. Examples of suitable prodrugs for this embodiment are provided in, e.g., Melton et al., Enzyme-Prodrug Strategies for Cancer Therapy, Kluwer Academic/Plenum Publishers, New York (1999), Bagshawe et al., Current Opinion in Immunology 11:579-83 (1999) and Kerr et A, Bioconjugate Chem. 9:255-59 (1998). In another embodiment, the CAB is specifically CAB1.6, CAB1.7 or CAB1.7i.

Examples of enzyme/prodrug/active drug combinations are found in, e.g., Bagshawe et al., Current Opinions in Immunology, 11:579-83 (1999); Wilman, “Prodrugs In Cancer Chemotherapy,” Biochemical Society Transactions, 14, pp. 375-82 (615th Meeting, Belfast 1986) and V. J. Stella et al., “Prodrugs: A Chemical Approach To Targeted Drug Delivery,” Directed Drug Delivery, R. Borchardt et al. (ed), pp. 247-67 (Humana Press 1985). In one embodiment, the prodrug is a peptide. Examples of peptides as prodrugs can be found in Trouet et al., Proc Natl Acad Sci USA 79:626 (1982), and Umemoto et al., Int J Cancer 43:677 (1989). These and other reports show that peptides are sufficiently stable in blood. Another advantage of peptide-derived prodrugs is their amino acid sequences can be chosen to confer suitable pharmacological properties like half-life, tissue distribution and low toxicity to the active drugs. Most reports of peptide-derived prodrugs relied on relatively nonspecific activation of the prodrug by, for instance, lysosomal enzymes.

The prodrug can be one that is converted to an active drug in more than one step. For example, the prodrug can be converted to a precursor of an active drug by the CAB. The precursor can be converted into the active drug by, for example, the catalytic activity of one or more additional CABs, the catalytic activities of one or more other enzymes administered to the subject, the catalytic activity of one or more enzymes naturally present in the subject or at the target site in the subject (e.g., a protease, a phosphatase, a kinase or a polymerase), by a drug that is administered to the subject or by a chemical process that is not enzymatically catalyzed (e.g., oxidation, hydrolysis, isomerization or epimerization).

Most studies involving prodrugs are generated after programs with existing drugs are found to be problematic. In particular anticancer drugs were generally characterized by a very low therapeutic index. By converting these drugs into prodrugs with reduced toxicity and then selectively activating them in the diseased tissue, the therapeutic index of the drug was significantly increased. See, e.g., Melton et al., Enzyme-prodrug strategies for cancer therapy (1999), and Niculescu-Duvaz et al., Anticancer Drug Des 14:517 (1999).

The literature describes many methods to alter the substrate specificity of enzymes by protein engineering or directed evolution. Thus one skilled in the art is able to evolve the specificity of an enzyme to accommodate even structures that would be poor substrates for naturally-occurring enzymes. Accordingly, prodrugs can be designed even though the drugs were otherwise not amenable to a prodrug strategy.

A number of studies have been performed with toxins coupled to targeting agents (usually antibodies or antibody fragments). See, e.g., Torchilin, Eur J Pharm Sci 11Suppl 2:S81 (2000) and Frankel et al., Clin Cancer Res 6:326 (2000). An alternative to the above is to convert these toxins into prodrugs and then selectively release them in the diseased tissue.

The prodrugs of this invention include, but are not limited to, aurstatins, camptothecins, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted by the enzyme of the conjugate into the more active cytotoxic free drug. Examples of cytotoxic drugs that can be derivatized into a prodrug form for use in this invention include, but are not limited to, etoposide, temposide, adriamycin, daunomycin, caminomycin, aminopterin, dactinomycin, mitomycins, cis-platinum and cis-platinum analogues, bleomycins, esperamicins (see U.S. Pat. No. 4,675,187), 5-fluorouracil, melphalan, other related nitrogen mustards and derivatives thereof. See, e.g., U.S. Pat. No. 4,975,278.

In one embodiment of the invention, the CAB comprises an alkaline phosphatase (AP) that converts a 4′-phosphate derivative of the epipodophyl-lotoxin glucosides into an active anti-cancer drug. Such derivatives include etoposide-4′-phosphate, etoposide-4′-thiophosphate and teniposide-4′-phosphate. Other embodiments of the invention may include phosphate derivatives of these glucosides wherein the phosphate moiety is placed at other hydroxyl groups on the glucosides. According to another embodiment, however, the phosphate derivative used as a pro-drug in this invention is etoposide-4′-phosphate or etoposide-4′-thiophosphate. The targeted AP removes the phosphate group from the prodrug, releasing an active antitumor agent. The mitomycin phosphate prodrug of this embodiment may be an N⁷—C₁₋₈ alkyl phosphate derivative of mitomycin C or porfiromycin or pharmaceutically acceptable salts thereof N⁷ refers to the nitrogen atom attached to the 7-position of the mitosane nucleus of the parent drug. According to another embodiment, the derivative used is 7-(2′-aminoethylphosphate)mitomycin (“MOP”). Alternatively, the MOP compound may be termed, 9-methoxy-7-[[(phosphonooxy)ethyl]amino]mitosane disodium salt. Other embodiments of the invention may include the use pfN⁷-allyl mitomycin phosphorothioates as prodrugs.

In still another embodiment of the invention, the CAB comprises a penicillin amidase enzyme that converts a novel adriamycin prodrug into the active antitumor drug adriamycin. In another embodiment, the penicillin amidase is a penicillin V amidase (“PVA”) isolated from Fusarium oxysporum that hydrolyzes phenoxyacetyl amide bonds. The prodrug utilized can be N-(p-hydroxyphenoxyacetyl)adriamycin (“APO”), which is hydrolyzed by the amidase to release the potent antitumor agent or adriamycin.

The present invention also comprises, for example, the use of the adriamycin prodrug, N-(p-hydroxyphenoxyacetyl)adriamycin and other related adriamycin prodrugs that can be derivatized in substantially the same manner. For example, use of the prodrug N-(phenoxyacetyl) adriamycin is also within the scope of the invention. In addition, it is to be understood that the adriamycin prodrugs of this invention include other N-hydroxyphenoxyacetyl derivatives of adriamycin, e.g., substituted at different positions of the phenyl ring, as well as N-phenoxyacetyl derivatives containing substituents on the phenyl ring other than the hydroxyl group described herein.

Furthermore, the present embodiment encompasses the use of other amidases, such as penicillin G amidase, as part of the CAB as well as other prodrugs correspondingly derivatized such that the particular amidase can hydrolyze that prodrug to an active antitumor form. For example, when the CAB further comprises penicillin G amidase, the prodrug should contain a phenylacetylamide group (as opposed to the phenoxyacetylamide group of APO) because penicillin G amidases hydrolyze this type of amide bond (see, e.g., A. L: Margolin et al., Biochim. Biophys Acta. 616, pp:283-89 (1980)). Thus, other prodrugs of the invention include N-(p-hydroxyphenylacetyl) adriamycin, N-(phenylacetyl) adriamycin and other optionally substituted N-phenylacetyl derivatives of adriamycin.

It should also be understood that the present invention includes any prodrug derived by reacting the amine group of the parent drug with the carboxyl group of phenoxyacetic acid, phenylacetic acid or other related acids. Thus, prodrugs of anthracyclines other than adriamycin that are capable of being derivatized and acting in substantially the same manner as the adriamycin prodrugs described herein falls within the scope of this invention. For example, other prodrugs that can be produced and used in accordance with this invention include hydroxyphenoxyacetylamide derivatives, bydroxyphenylacetylamide derivatives, phenoxyacetylamide derivatives and phenylacetylamide derivatives of anthracyclines such as daunomycin and caminomycin. Other amine-containing drugs such as melphalan, mitomycin, aminopterin, bleomycin and dactinomycin can also be modified described herein to yield prodrugs of the invention.

Another embodiment of the invention involves a CAB form of the enzyme cytosine deaminase (“CD”). The deaminase enzyme catalyzes the conversion of 5-fluorocytosine (“5-FC”), a compound lacking in antineoplastic activity, to the potent antitumor drug, 5-fluorouracil (“5-FU”).

Another embodiment of the method of this invention provides a method of combination chemotherapy using several prodrugs and a single CAB. According to this embodiment, a number of prodrugs are used that are all substrates for the same CAB. Thus, a particular CAB converts a number of prodrugs into cytotoxic form, resulting in increased antitumor activity at the tumor site.

There is often a requirement for extending the blood circulation half-lives of pharmaceutical peptides, proteins, or small molecules. Typically short half-lives—lasting minutes to hours—require not only frequent, but also high doses for therapeutic effect—often so high that initial peak doses cause side effects. Extending the half-life of such therapeutics permits lower, less frequent, and therefore potentially safer doses, which are cheaper to produce. Previously researchers have increased protein half-life by fusig them-covalently to PEG, see U.S. Pat. No. 5,711,944, human blood serum albumin, see U.S. Pat. No. 5,766,883, or Fc fragments, see WO 00/24782. In addition, nonspecific targeting of drugs to human serum albumin has been accomplished by chemical coupling drugs in vivo. See U.S. Pat. No. 5,843,446. Furthermore, in the case of cancer drugs it has been proposed that high molecular weight drugs may localize in tumors due to enhanced permeability and retention. Therefore, improvement in the therapeutic index of a drug can be obtained by linking the drug to a protein or other high molecular weight polymer.

In another embodiment the present invention provides a method of treating a condition in subject comprising administering to the subject a CAB with β-lactamase activity and a prodrug. In another embodiment, the CAB is targeted to a CEA expressing cell, tissue, tumor or organ. In another embodiment, the prodrug is converted by the CAB into an active drug. In another embodiment, the active drug is an alkylating agent. In another embodiment, the prodrug is an anticancer nitrogen mustard prodrug. In another embodiment, the active drug is melphalan. In another embodiment, the prodrug is glutaryl-C-Mel or glutaryl-C-Mel-L-Phe-NH2 (see, for example, Senter et al, U.S. Pat. No. 5,773,435, which is incorporated by reference herein, including any drawings and Kerr et al., Bioconjugate Chem. 9:255-59 (1998)). In another embodiment, the prodrug is C-Mel. See Kerr et al., Bioconjugate Chem. 9:255-59 (1998). In another embodiment, the prodrug is vinca-cephalosporin or doxorubicin cephalosporin. See Bagshawe et al., Current Opinion in Immunology, 11:579-83 (1999). Other prodrug/enzyme combinations that can be used in the present invention include, but are not limited to, those found in U.S. Pat. No. 4,975,278 and Melton et al., Enzyme-Prodrug Strategies for Cancer Therapy Kluwer Academic/Plenum Publishers, New York (1999).

In a fourth aspect, the invention is drawn to a pharmaceutical composition comprising a CAB molecule. The CABs, nucleic acids encoding them and, in certain embodiments, prodrugs described herein can be incorporated into pharmaceutical compositions suitable for administration. Such compositions typically comprise the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a CAB, prodrug or nucleic acid of interest. Such methods comprise formulating a pharmaceutically acceptable carrier with an agent that modulates expression or activity of an active compound of interest. Such compositions can further include additional active agents. Thus, the invention further includes methods for preparing a pharmaceutical composition by formulating a pharmaceutically acceptable carrier with an agent that modulates expression or activity of a CAB, prodrug or nucleic acid of interest and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or so sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars; polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound Can be incorporated with excipients and used in the form of tablets, troches or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring.

For administration by inhalation, the compounds are delivered in the form of an aerosol spray from a pressurized container or dispenser that contains a suitable propellant, e.g., a gas such as carbon dioxide or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved and the limitations inherent in the art of compounding such an active compound for the treatment of individuals.

Typically, the amount of CAB to be delivered to a subject will depend on a number of factors, including, for example, the route of administration, the activity of the CAB, the degree to which it is specifically targeted to the desired cells, tissues or organs of the subject, the length of time required to clear the non-specifically bound CAB from the subject, the desired therapeutic effect, the body mass of the subject, the age of the subject, the general health of the subject, the sex of the subject, the diet of the subject, the subject's immune response to the CAB, other medications or treatments being administered to the subject, the severity of the disease and the previous or future anticipated course of treatment.

For applications in which a prodrug also is administered, other factors affecting the determination of a therapeutically effective dose will include, for example, the amount of prodrug administered, the activity of the prodrug and its corresponding active drug and the side effects or toxicities of the prodrug and the active drug.

Examples of ranges of mass of CAB/mass of subject include, for example, from about 0.001 to 30 mg/kg body weight, from about 0.01 to 25 mg/kg body weight, from about 0.1 to 20 mg/kg body weight, and from about 1 to 10 mg/kg, 2 to 9 mg/kg, 3 to 8 mg/kg, 4 to 7 mg/kg, or 5 to 6 mg/kg body weight.

In a particular example, a subject is treated with a CAB in the range of between about 0.1 to 20 mg/kg body weight, one time per week for between about 1 to 10 weeks, preferably between 2 to 8 weeks, preferably between about 3 to 7 weeks and preferably for about 4, 5, or 6 weeks. It will also be appreciated that the effective dosage of CAB may increase or decrease over the course of a particular treatment, and that the treatment will continue, with or without modification, until a desired result is achieved or until the treatment is discontinued for another reason. Changes in dosage may result and become apparent from the results of diagnostic assays as described herein.

In an embodiment of the present invention, prodrug also is administered to the subject. It is understood that appropriate doses of prodrugs depend upon a number of factors within the ken of the ordinarily skilled physician, veterinarian or researcher. The dose(s) of the prodrug will depend, for example, on the same factors provided above as factors affecting the effective dose of the CAB. Exemplary doses include milligram or microgram amounts of the prodrug per kilogram of subject or sample weight (e.g., about 1 microgram per kilogram to about 500 milligrams per kilogram, about 100 micrograms per kilogram to about 5 milligrams per kilogram or about 1 microgram per kilogram to about 50 micrograms per kilogram). It is furthermore understood that appropriate doses of a prodrug depend upon the potency of the prodrug with respect to the desired therapeutic effect. When one or more of these prodrugs is to be administered to an animal (e.g., a human), a physician, veterinarian or researcher may, for example, prescribe a relatively low dose at first, subsequently increasing the dose until an appropriate response is obtained.

Preferably, the CAB is administered to the subject, then the prodrug is administered. More preferably, the time between the administration of the CAB and administration of the prodrug is sufficient to allow the CAB to accumulate at its target site by binding to its target, and to allow unbound CAB to be cleared from the non-targeted portions of the subject's body. Most preferably, the ratio of target-bound CAB to unbound CAB in the subject's body will be at or near its maximum when the prodrug is administered. The time necessary after administration of the CAB to reach this point is called the clearing time. The clearing time can be determined or approximated in an experimental system by, for example, administering a detectable CAB (e.g., a radiolabeled or fluorescently labeled CAB) to a subject and simultaneously measuring the amount of enzyme at the target site and at a non-targeted control site at timed intervals. For some prodrugs, particularly those whose counterpart active drugs are highly toxic, it may be more important to ensure that the levels of unbound CAB in the subject's system are below a certain threshold. This too can be determined experimentally, as described above.

In one embodiment, administration of the prodrug is systemic. In another embodiment, administration of the prodrug is at or near the target to be bound.

The pharmaceutical compositions can be included in a container, pack, dispenser or kit together with instructions for administration.

### EXAMPLES

### Example 1

### Stabilization of an scFv

Construction of pME27:1

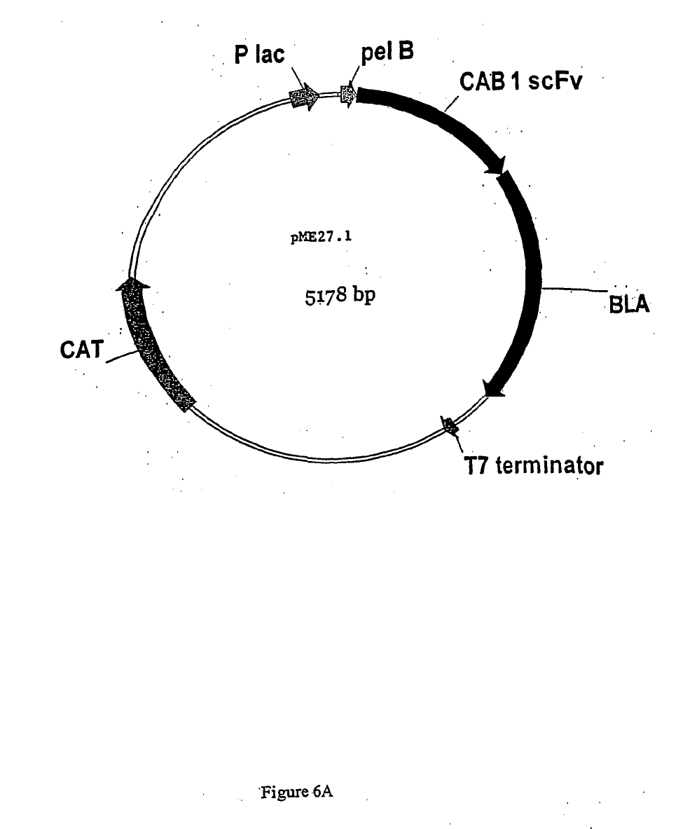

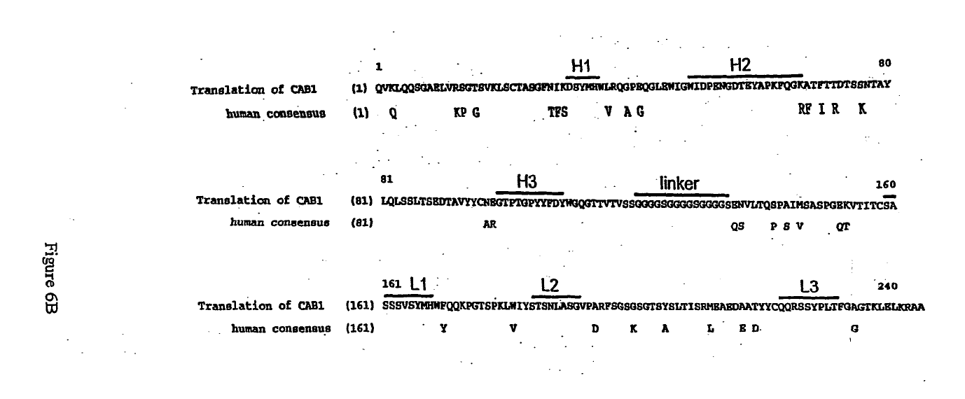

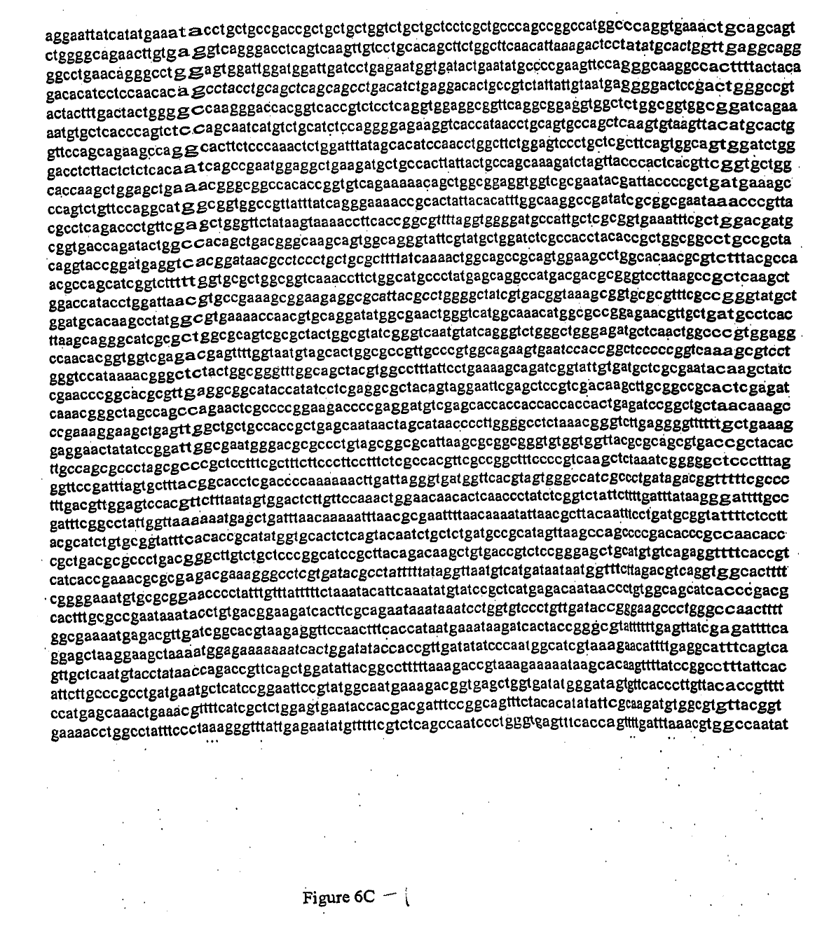

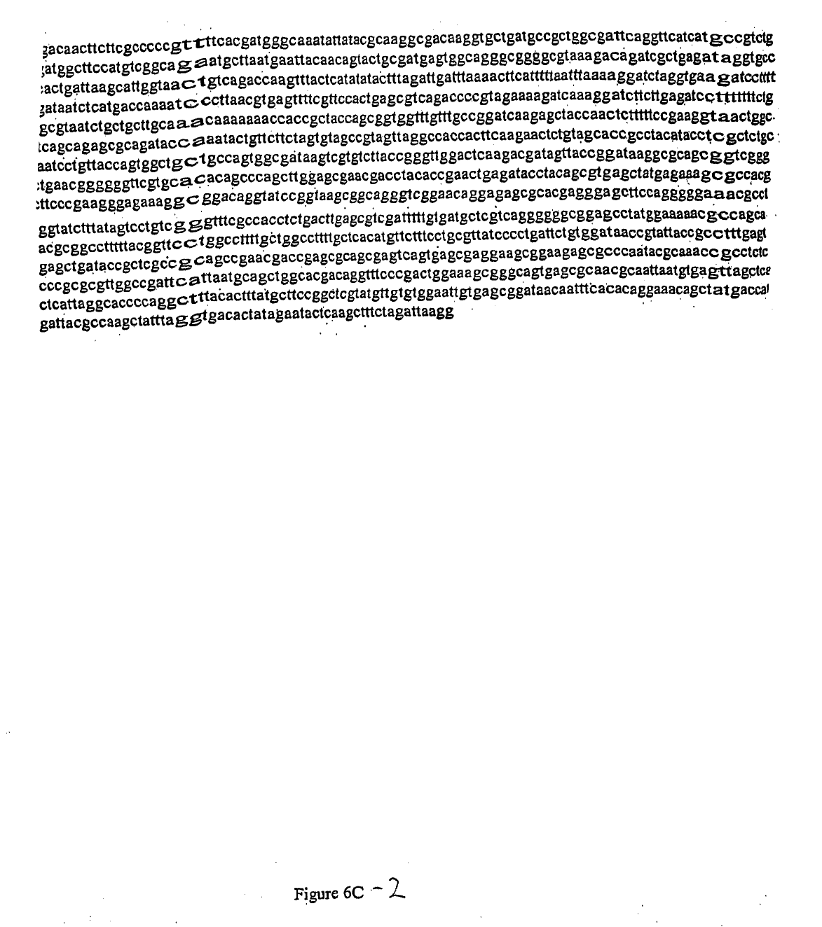

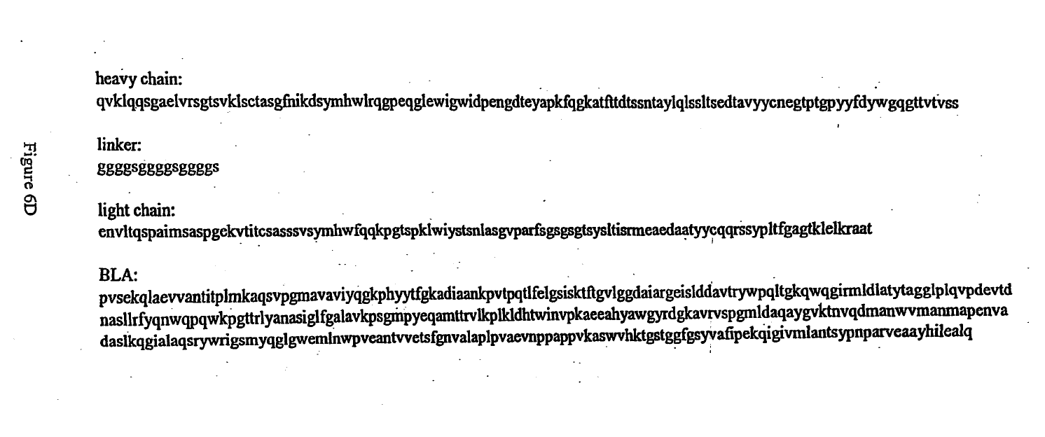

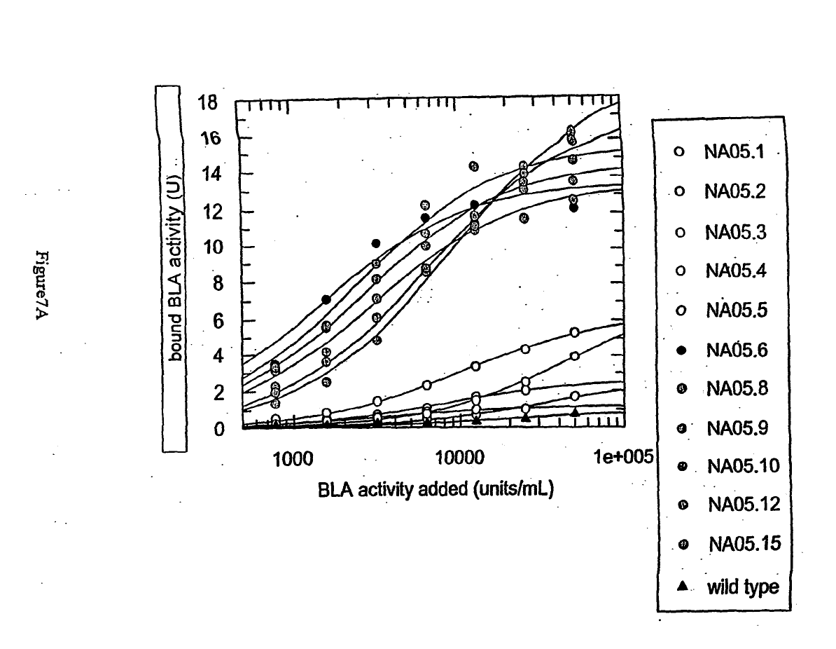

Plasmid pME27.1 was generated by inserting a Bgl I-EcoRV fragment encoding a part of the pelB leader, the CAB1-scFv and a small part of BLA into the expression vector pME25 (see, FIG. 6). The insert, encoding for the CAB1-scFv has been synthesized by Aptagen (Hemdon, Va.) based on the sequence of the scFv MFE-23 that was described in [Boehm, M. K., A. L. Cover, T. Wan, M. K. Sohi, B. J. Sutton, J. D. Thornton, P. A. Keep, K. A. Chester, R. H. Begent and S. J. Perkins (2000) Biochem J 346 Pt 2, 519-28, Crystal structure of the anti-(carcinoembryonic antigen) single-chain Fv antibody MFE-23 and a model for antigen binding based on intermolecular contacts]. Both the plasmid containing the synthetic gene (pPCR-GME1) and pME25 were digested with BglI and EcoRV, gel purified and ligated together with Takara ligase. Ligation was transformed into TOP10 (Invitrogen, Carlsbad, Calif.) electrocompetent cells, plated on LA medium containing 5 mg/l chloramphenicol and 0.1 mg/l cefotaxime.

Plasmid pME27.1 contains the following features:

| P lac: | 4992-5113 bp |
|---|---|
| pel B leader: | 13-78 |
| CAB 1 scFv: | 79-810 |
| BLA: | 811-1896 |
| T7 term.: | 2076-2122 |
| CAT: | 3253-3912 |

A schematic of plasmid pME27.1 can be found in FIG. 6A. The CAB1 sequence, indicating heavy and light chain domains, can be found in FIG. 6B; the amino acid sequence can also be found in FIG. 6D, with linker and BLA.

### Choosing Mutations for Mutagenesis

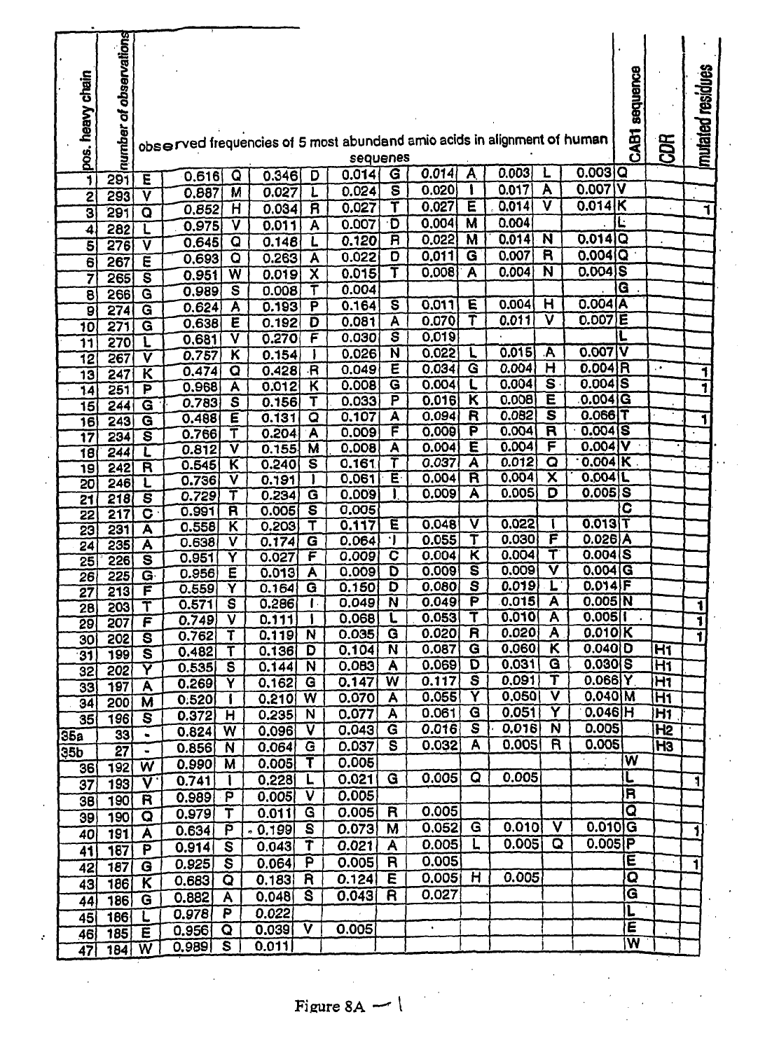

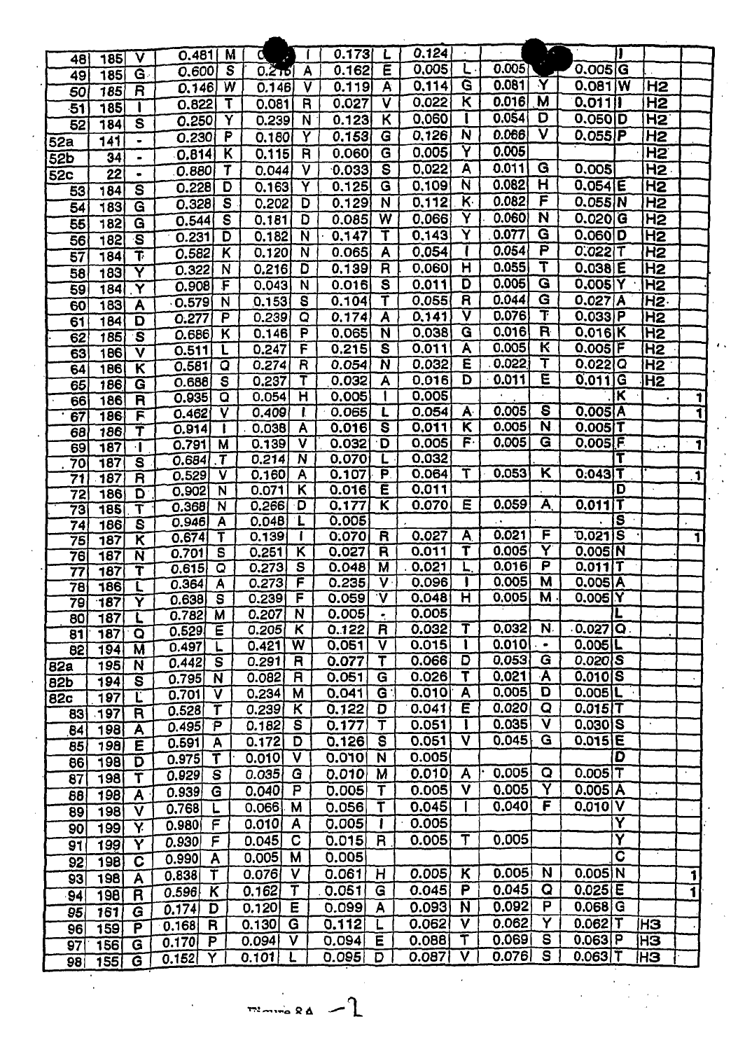

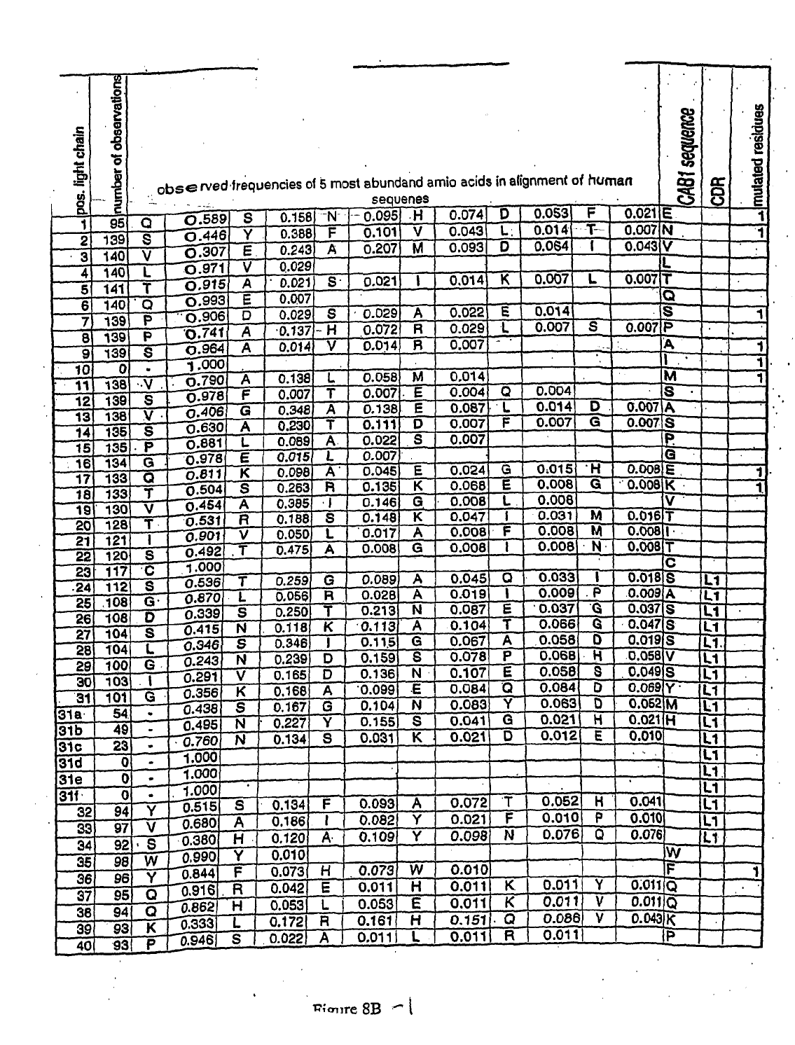

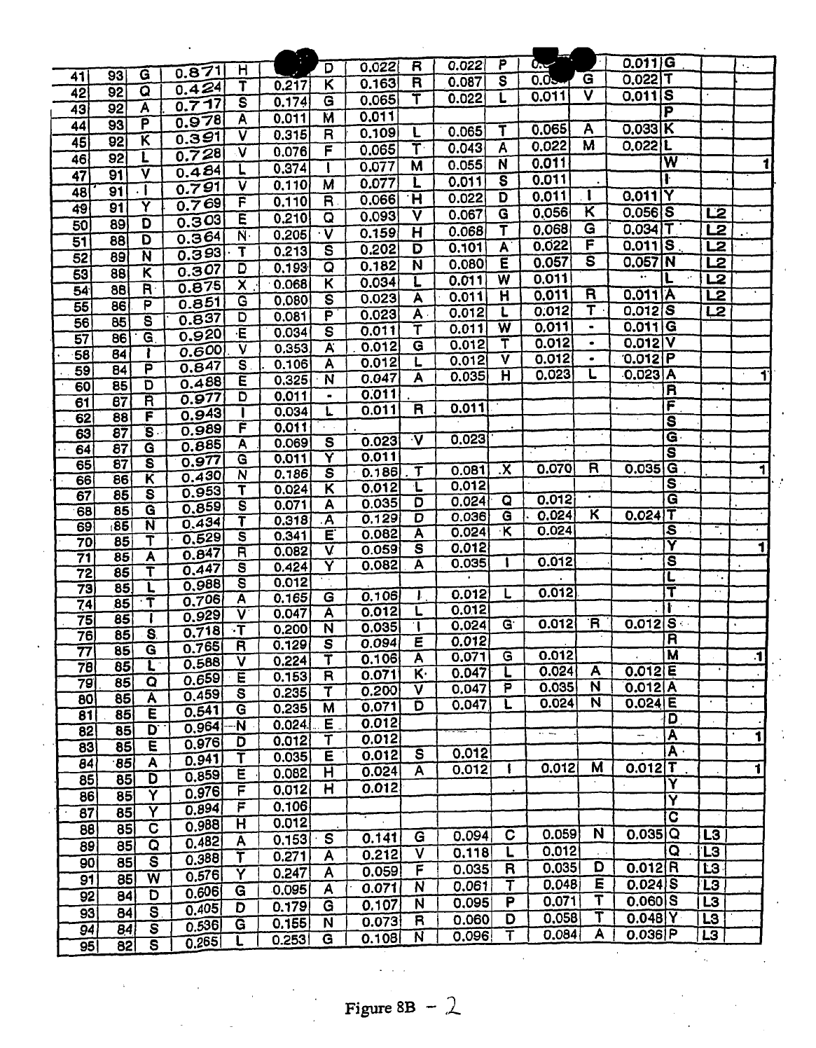

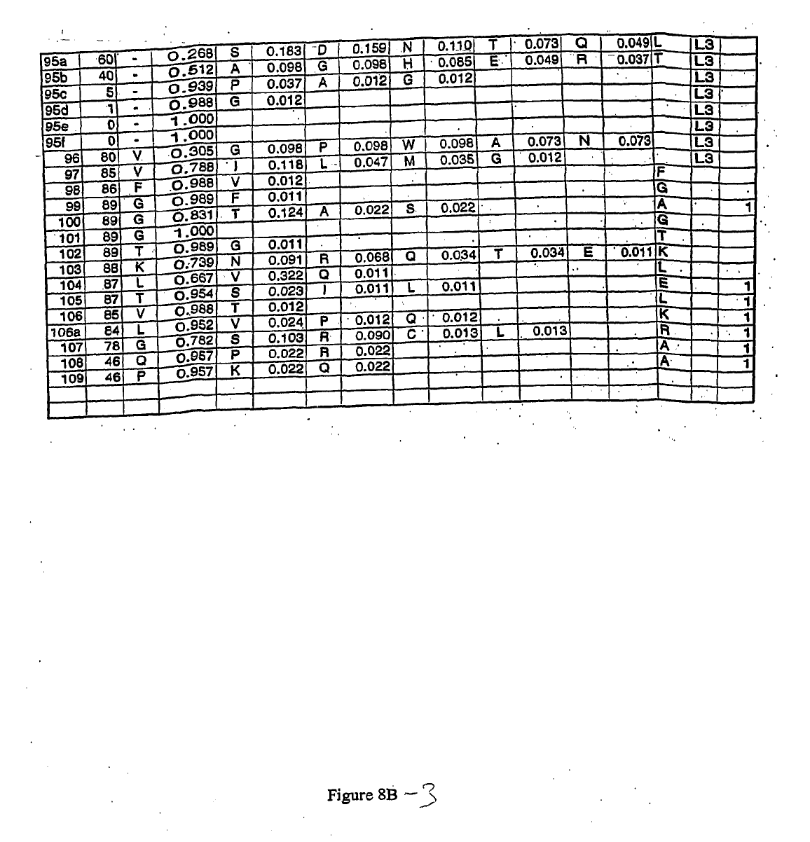

The sequence of the vH and vL sequences of CAB1-scFv were compared with a published frequency analysis of human antibodies (Boris. Steipe (1998) Sequenzdatenanalyse. (“Sequence Data Analysis”, available in German only) in Bioanalytik eds. H. Zorbas and F. Lottspeich, Spektrum Akademischer Verlag. S. 233-241). The authors aligned sequences of variable segments of human antibodies as found in the Kabat data base and calculated the frequency of occurrence of each amino acid for each position. These alignments can be seen in FIG. 8. Specifically, FIG. 8A shows an alignment of the observed frequencies of the five most abundant amino acids in alignment of human sequences in the heavy chain. FIG. 8B shows an alignment of the observed frequencies of the five most abundant amino acids in alignment of human sequences in the light chain.

We compared these frequencies with the actual amino acid sequence of CAB1 and identified 33 positions that fulfilled the following criteria:
The position is not part of a CDR as defined by the Kabat nomenclature.
The amino acid found in CAB1-scFv is observed in the homologous position in less than 10% of human antibodies
The position is not one of the last 6 amino acids in the light chain of scFv.
   The resulting 33 positions were chosen for combinatorial mutagenesis.

Mutagenic oligonucleotides were synthesized for each of the 33 positions such that the targeted position would be changed from the amino acid in CAB1-scFv to the most abundant amino acid in the homologous position of a human antibody. FIG. 6B shows the sequence of CAB1-scFv, the CDRs and the mutations that were chosen for combinatorial mutagenesis.

### Construction of Library NA05

Table 1 listing the sequences of 33 mutagenic oligonucleotides that were used to generate combinatorial library NA05:

TABLE 1

| pos. | MFE-23 | count residues | |
|---|---|---|---|
| (pME27) | (VH) | to be changed | QuikChange multi primer |
| 3 K | Q | nsa147.1fp | CGGCCATGGCCCAGGTGCAGCTGCAGCAGTCTGGGGC |
| 13 R | K | nsa147.2fp | CTGGGGCAGAACTTGTGAAATCAGGGACCTCAGTCAA |
| 14 S | P | nsa147.3fp | GGGCAGAACTTGTGAGGCCGGGGACCTCAGTCAAGTT |
| 16 T | G | nsa147.4fp | AACTTGTGAGGTCAGGGGGCTCAGTCAAGTTGTCCTG |
| 28 N | T | nsa147.5fp | GCACAGCTTCTGGCTTCACCATTAAAGACTCCTATAT |
| 29 I | F | nsa147.6fP | CAGCTTCTGGCTTCAACTTTAAAGACTCCTATATGCA |
| 30 K | S | nsa147.7fp | CTTCTGGCTTCAACATTAGCGACTCCTATATGCACTG |
| 37 L | V | nsa147.8fp | ACTCCTATATGCACTGGGTGAGGCAGGGGCCTGAACA |
| 40 G | A | nsa147.9fp | TGCACTGGTTGAGGCAGGCGCCTGAACAGGGCCTGGA |
| 42 E | G | nsa147.10fp | GGTTGAGGCAGGGGCCTGGCCAGGGCCTGGAGTGGAT |
| 67 K | R | nsa147.11fp | CCCCGAAGTTCCAGGGCCGTGCCACTTTTACTACAGA |
| 68 A | F | nsa147.12fp | CGAAGTTCCAGGGCAAGTTCACTTTTACTACAGACAC |
| 70 F | I | nsa147.13fp | TCCAGGGCAAGGCCACTATTACTACAGACACATCCTC |
| 72 T | R | nsa147.14fp | GCAAGGCCACTTTTACTCGCGACACATCCTCCAACAC |
| 76 S | K | nsa147.15fp | TTACTACAGACACATCCAAAAACACAGCCTACCTGCA |
| 97 N | A | nsa147.16fp | CTGCCGTCTATTATTGTGCGGAGGGGACTCCGACTGG |
| 98 E | R | nsa147.17fp | CCGTCTATTATTGTAATCGCGGGACTCCGACTGGGCC |
| 136 E | Q | nsa147.18fp | CTGGCGGTGGCGGATCACAGAATGTGCTCACCCAGTC |
| 137 N | S | nsa147.19fp | GCGGTGGCGGATCAGAAAGCGTGCTCACCCAGTCTCC |
| 142 S | P | nsa147.20fp | GAAAATGTGCTCACCCAGCCGCCAGCAATCATGTCTGC |
| 144 A | S | nsa147.21fp | TGCTCACCCAGTCTCCAAGCATCATGTCTGCATCTCC |
| 146 M | V | nsa147.22fp | CCCAGTCTCCAGCAATCGTGTCTGCATCTCCAGGGGA |
| 152 E | Q | nsa147.23fp | TGTCTGCATCTCCAGGGCAGAAGGTCACCATAACCTG |
| 153 K | T | nsa147.24fp | CTGCATCTCCAGGGGAGACCGTCACCATAACCTGCAG |
| 170 F | Y | nsa147.25fp | TAAGTTACATGCACTGGTACCAGCAGAAGCCAGGCAC |
| 181 W | V | nsa147.26fp | GCACTTCTCCCAAACTCGTGATTTATAGCACATCCAA |
| 194 A | D | nsa147.27fp | TGGCTTCTGGAGTCCCTGATCGCTTCAGTGGCAGTGG |
| 200 G | K | nsa147.28fp | CTCGCTTCAGTGGCAGTAAATCTGGGACCTCTTACTC |
| 205 Y | A | nsa147.29fp | GTGGATCTGGGACCTCTGCGTCTCTCACAATCAGCCG |
| 212 M | L | nsa147.30fp | CTCTCACAATCAGCCGACTGGAGGCTGAAGATGCTGC |
| 217 A | E | nsa147.31fp | GAATGGAGGCTGAAGATGAAGCCACTTATTACTGCCA |
| 219 T | D | nsa147.32fp | AGGCTGAAGATGCTGCCGA1TATTACTGCCAGCAAAG |
| 234 A | G | nsa147.33fp | ACCCACTCACGTTCGGTGGCGGCACCAAGCTGGAGCT |

The QuikChange multi site-directed mutagenesis kit (QCMS; Stratagene Catalog #200514) was used to construct the combinatorial library NAOS using 33 mutagenic primers. The primers were designed so that they had 17 bases flanking each side of the codon of interest based on the template plasmid pME27.1. The codon of interest was changed to encode the appropriate consensus amino acid using an E. coli codon usage table. All primers were designed to anneal to the same strand of the template DNA (i.e., all were forward primers in this case). The QCMS reaction was carried out as described in the QCMS manual with the exception of the primer concentration used; the QCMC manual recommends using 50 ng of each primer in the reaction, whereas we used 3 ng of each primer. Other primer amounts may be used. In particular, the reaction contained 50-100 ng template plasmid (pME27.1; 5178 bp), 1 μl of primer mix (10 μM stock of all primers combined containing 0.3 μM each primer), 1 μl dNTPs (QCMS kit), 2.5 μl 10×QCMS reaction buffer, 18.5 μl deoinized water and 1 μl enzyme blend (QCMS kit) for a total volume of 25 μl. The thermocycling program was 1 cycle at 95° C. for 1 min., followed by 30 cycles of 95° C. for 1 min., 55° C. for 1 min. and then 65° C. for 10 minutes. DpnI digestion was performed by adding 1 μl DpnI, (provided in the QCMS kit), incubation at 37° C. for 2 hours, addition of another 1 μl Dpn1, and incubation at 37° C. for an additional 2 hours. 1 μl of the reaction was transformed into 50 μl of TOP10 electrocompetent cells from Invitrogen. 250 μl of SOC was added after electroporation, followed by a 1 hr incubation with shaking at 37° C. Thereafter, 10-50 μl of the tranformation mix was plated on LA plates with 5 ppm chloramphenicol (CMP) or LA plates with 5 ppm CMP and 0.1 ppm of cefotaxime (CTX) for selection of active BLA clones. The active BLA clones from the CMP+CTX plates were used for screening whereas the random library clones from the CMP plates were sequenced to assess the quality of the library.

16 randomly chosen clones were sequenced. The clones contained different combinations of 1 to 7 mutations.

### Screen for Improved Expression

When TOP10/pME27.1 is cultured in LB medium at 37 C then the concentration of intact fusion protein peaks after one day and most of the fusion protein is degraded by host proteases after 3 days of culture. Degradation seems to occur mainly in the scFv portion of the CAB1 fusion protein as the cultures contain significant amounts of free BLA after 3 days, which can be detected by Western blotting, or a nitrocefin (Oxoid, N.Y.) activity assay. Thus we applied a screen to library NA05 that was able to detect variants of CAB1-scFv that would resist degradation by host proteases over 3 days of culture at 37 C.

Library NA05 was plated onto agar plates with LA medium containing 5 mg/l chloramphenicol and 0.1 mg/l cefotaxime (Sigma). 910 colonies were transferred into a total of 10 96-well plates containing 100 ul/well of LA medium containing 5 mg/l chloramphenicol and 0.1 mg/l cefotaxime. Four wells in each plate were inoculated with TOP10/pME27.1 as control and one well per plate was left as a blank. The plates were grown overnight at 37 C. The next day the cultures were used to inoculate fresh plates (production plates) containing 100 ul of the same medium using a transfer stamping tool and glycerol was added to the master plates which were stored at −70 C. The production plates were incubated in a humidified shaker at 37 C for 3 days. 100 ul of BPER (Pierce, Rockford, Ill.) per well was added to the production plate to release protein from the cells. The production plate was diluted 100-fold in PBST (PBS containing 0.125% Tween-20) and BLA activity was measured by transferring 20 ul diluted lysate into 180 ul of nitrocephin assay buffer (0.1 mg/ml nitrocephin in 50 mM PBS buffer containing 0.125% octylglucopyranoside (Sigma)) and the BLA activity was determined at 490 inn using a Spectramax plus plate reader (Molecular Devices, Sunnyvale, Calif.).

Binding to CEA (carcinoembryonic antigen, Biodesign Intl., Saco, Me.) was measured using the following procedure: 96-well plates were coated with 100 ul per well of 5 ug/ml of CEA in 50 mM carbonate buffer pH 9.6 overnight. The plates were washed with PBST and blocked for 1-2 hours with 300 ul of casein (Pierce, Rockford, Ill.). 100 ul of sample from the production plate diluted 100-1000 fold was added to the CEA-coated plate and the plates were incubated for 2 h at room temperature. Subsequently, the plates were washed four times with PBST and 200 ul nitrocefin assay buffer was added, and the BLA activity was measured as described above.

The BLA activity that was determined by the CEA-binding assay and the total BLA activity found in the lysate plates were compared and variants were identified that showed high levels of total BLA activity and high levels of CEA-binding activities.

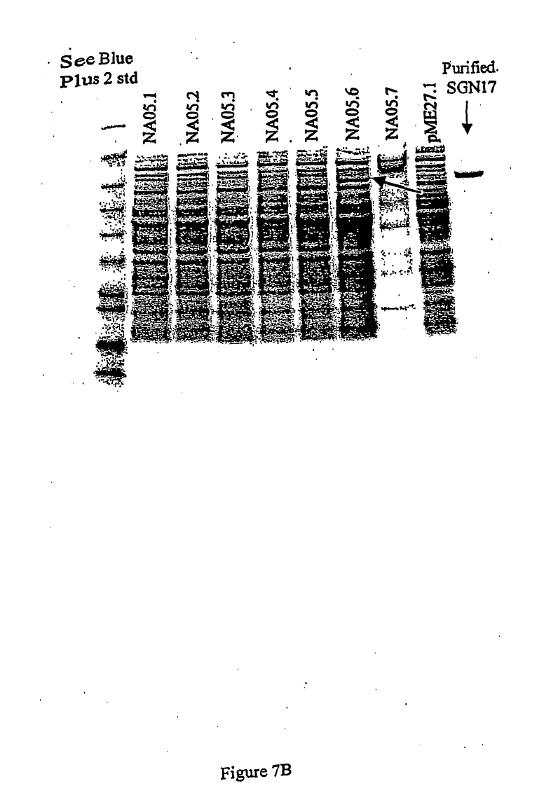

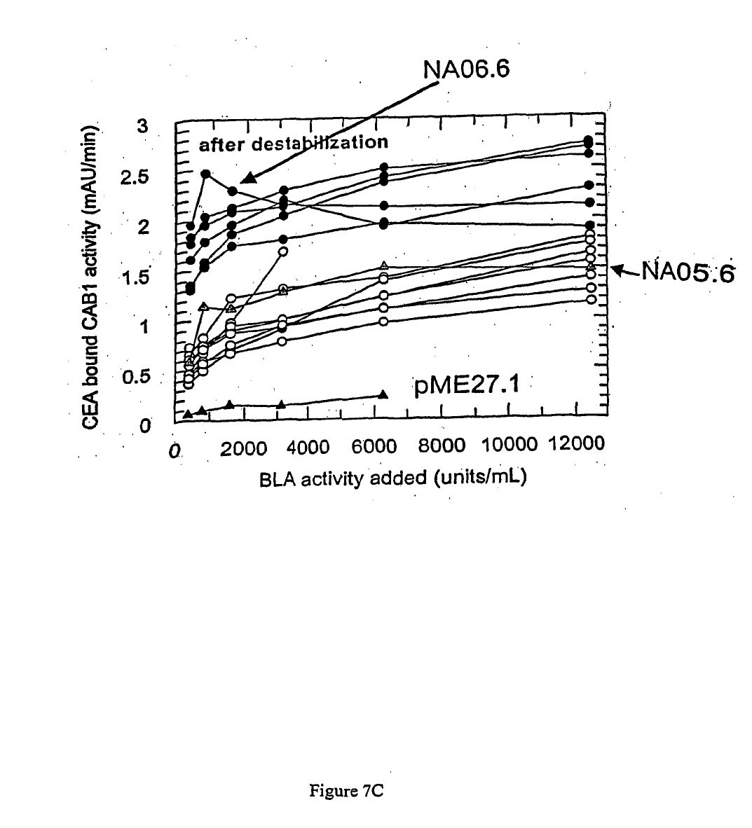

The winners were confirmed in 4 replicates using a similar protocol: the winners were cultured in 2 ml of LB containing 5 mg/l chloramphenicol and 0.1 mg/l cefotaxime for 3 days. Protein was released from the cells using BPER reagent. The binding assay was performed as described above but different dilutions of culture lysate were tested for each variant FIG. 7A shows binding curves. Culture supernatants were also analyzed by SDS polyacrylamide electrophoresis. FIG. 7B shows the electropherogram of 7 variants from NA05. The band of the fusion protein is labeled for variant NA05.6. Table 2 shows a ranking of 6 variants. The data were normalized and a performance index was calculated. The data clearly show that NA05.6 produces significantly larger quantities of fusion protein compared to the fusion construct pME27.1.

Table 2 showing the sequence of 6 variants with the largest improvement in stability:

| Clone | mutations |
|---|---|
| NA05.6 | R13K, T16G, W181V |
| NA05.8 | R13K, F170Y, A234G |
| NA05.9 | K3Q, S14P, L37V, E42G, E136Q, |
| | M146V, W181V, A234G |
| NA05.10 | K3Q, L37V, P170Y, W181V |
| NA05.12 | K3Q, S14P, L37V, M146V |
| NA05.15 | M146V, F170Y, A194D |

### Construction of Library NA06

Clone NA05.6 was chosen as the best variant and was used as the template for a second round of combinatorial mutagenesis; clone NA05.6 was designated CAB1.1. A subset of the same mutagenic primers that had been used to generate library NA05 to generate combinatorial variants with the following mutations: K3Q, L37V, E42G, E136Q, M146V, F170Y, A194D, A234G, was used; the mutations had been identified in other winners from library NA05. The primer encoding mutation S14P was not used as its sequence overlapped with mutations R13K and T16G present in NA05.6 (CAB1.1). A combinatorial library was constructed using QuikChange Multisite as described above and was called NA06. The template was pNA05.6 and 1 μl of primers mix (10 μM stock of all primers combined containing 1.25 μM each primer) were used:

### Screening of Library NA06

The screen was performed as described above with the following modifications: 291 variants were screened on 3 96-well plates. 10 μl sample from the lysate plates was added to 180 μl of 10 μg/ml thermolysin (Sigma) in 50 mM imidazole buffer pH 7.0 containing 0.005% Tween-20 and 10 mM calcium chloride. This mixture was incubated for 1 h at 37 C to hydrolyze unstable variants of NA05.6 (CAB1.1). This protease-treated sample was used to perform the CEA-binding assay as described above.

Promising variants were cultured in 2 ml medium as described above and binding curves were obtained for samples after thermolysin treatments. FIG. 7C shows binding curves for selected clones. A number of variants retain much more binding activity after thermolysin incubation than the parent NA05.6 (CAB1.1)

Table 3 shows 6 variants significantly more protease resistant than NA05.6 (CAB1.1):

| Clone | Mutations |
|---|---|
| NA06.2 | R13K, T16G, W181V, L37V, E42G, |
| | A194D |
| NA06.4 | R13K, T16G, W181V, L37V, M146V |
| NA06.6 | R13K, T16G, W181V, L37V, M146V, |
| | K3Q |
| NA06.10 | R13K, T16G, W181V, L37V, M146V, |
| | A194D |
| NA06.11 | R13K, T16G, W181V, L37V, K3Q, A194D |
| NA06.12 | R13K, T16G, W181V, L37V, E136Q |

All 6 variants have the mutation L37V; the mutation was rare in randomly chosen clones from the same library. Further testing showed that variant NA06.6 had the highest level of total BLA activity and the highest protease resistance of all variants. NA06.6 was chosen and designated CAB1.2.

### Example 2

### Generation of an scFV that has pH-Dependent Binding Choosing Positions for Mutagenesis

The 3D structure of the scFv portion of NA06.6 (CAB1.2) was modeled based on the published crystal structure of a close homologue, MFE-23 [Boehm, M. K., A. L. Corper, T. Wan, M. K. Sohi, B. J. Sutton, J. D. Thornton, P. A. Keep, K. A. Chester, R. H. Begentind S. J. Perkins (2000) Biochem J 346 Pt 2 519-28, Crystal structure of the anti-(carcinoembryonic antigen) single-chain Fv antibody MFE-23 and a model for antigen binding based on intermolecular contacts] using the software package MOE (Chemical Computing Group, Montreal, Canada) and using default parameters. A space-filling model of the structure was visually inspected. Side chains in the CDRs were ranked as follows: 0=buried, 1=partially exposed and 2=completely exposed. Side chain distance to CDR3 was ranked as follows: 0=side chain is in CDR3, 1=side chain is one amino acid away from CDR3 and 2=side chain is two amino acids away from CDR3. In a few cases, residues flanking the CDRs were included if they fit the distance and exposure criteria.

Based on this ranking, the following side chains were targeted for mutagenesis:
a) exposure=2 and distance=2 or smaller
b) exposure=1 and distance <2
   40 positions in the CDRs matched these criteria.

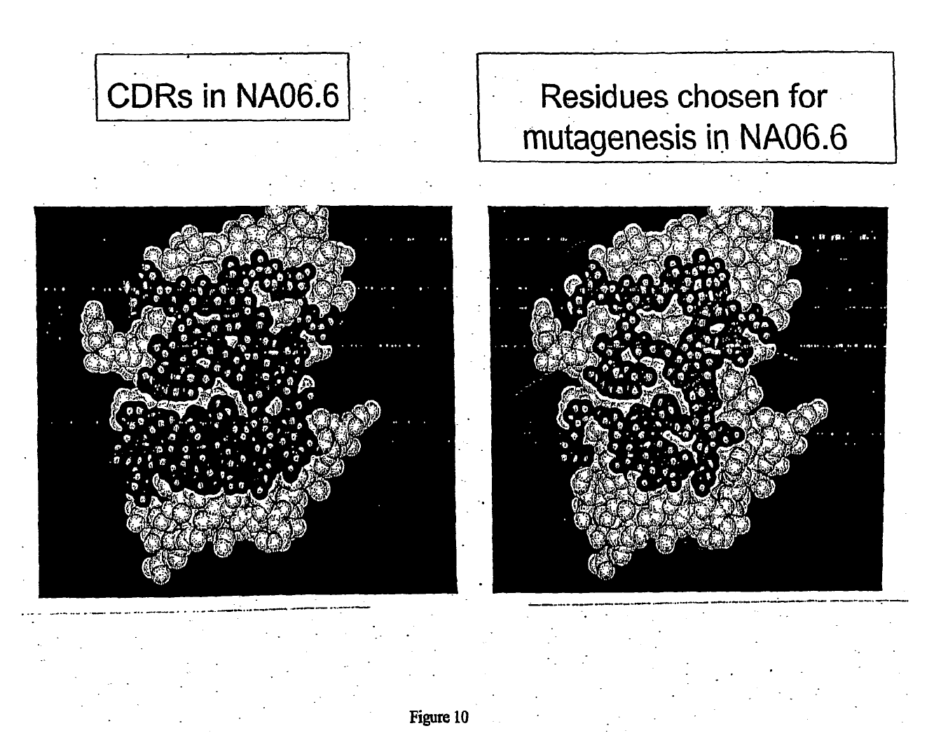

FIG. 10 shows the CDRs and the residues that were chosen for mutagenesis.

Table 4 shows the criteria and position of the 40 sites that were chosen for mutagenesis.

### Construction of Library NA08

A combinatorial library was constructed where the 40 selected positions were randomly replaced with aspartate or histidine. The substitutions were chosen as it has been reported that ionic interactions between histidine side chains and carboxyl groups form the structural basis for the pH-dependence of the interaction between IgG molecules and the Fc receptor [Vaughn, D. E. and P. J. Bjorkman (1998) Structure 6, 63-73., Structural basis of pH-dependent antibody binding by the neonatal Fc receptor].

The QuikChange multi site directed mutagenesis kit (QCMS; Stratagene Catalog #200514) was used to construct the combinatorial library NA08 using 40 mutagenic primers. The primers were designed so that they had 17 bases flanking each side of the codon of interest based on the template plasmid NA06.6 (CAB1:2). The codon of interest was changed to the degenerate codon SAT to encode for aspartate and histidine. All primers were designed to anneal to the same strand of the template DNA (i.e., all were forward primers in this case). The QCMS reaction was carried out as described in the QCMS manual with the exception of the primer concentration used; the manual recommends using 50-100 ng of each primer in the reaction, whereas significantly lower amounts of each primer were used in this library as this results in a lower parent template background. In particular, 0.4 μM of all primers together were used. The individual degenerate primer concentration in the final reaction was 0.01 μM (approximately 2.5 ng).

The QCMS reaction contained 50-100 ng template plasmid (NA06.6, 5178 bp), 1 μl of primer mix (10 μM stock of all primers to give the desired primer concentration mentioned above), 1 μl dNTPs (QCMS kit), 2.5 μl 10×QCMS reaction buffer, 18.5 μl deoinized water, and 1 μl enzyme blend (QCMS kit), for a total volume of 25 μl. The thermocycling program was 1 cycle at 95° C. for 1 min., followed by 30 cycles of 95° C. for 1 min., 55° C. for 1 min. and 65° C. for 10 minutes. DpnI digestion was performed by adding 1 μl DpnI (provided in the QCMS kit), incubating at 37° C. for 2 hours, adding of 0.5 μl DpnI and then incubating at 37° C. for an additional 2 hours. 1 μl of each reaction was transformed into 50 μl of TOP10 electrocompetent cells from Invitrogen. 250 μl of SOC was added after electroporation, followed by a 1 hr incubation with shaking at 37° C. Thereafter, 10-50 μl of the transformation mix was plated on LA plates with 5 ppm chloramphenicol (CMP) or LA plates with 5 ppm CMP and 0.1 ppm of cefotaxime (CTX) for selection of active BLA clones. The number of colonies obtained on both types of plates was comparable (652 on the CMP plate and 596 colonies on the CMP+CTX plate for 10 μl of the transformation mix plated). Active BLA clones from the CMP+CTX plates were used for screening, whereas random library clones from the CMP plates were sequenced to assess the quality of the library.

Primers for the reaction are shown in Table 4:

TABLE 4
Primers for CDRs:

| | | position | distance to | |
|---|---|---|---|---|
| Residue | CDRs | exposure | CDR3 | primer sequence |
| K | | 30 | 2 | 2 cttctggcttcaacattsatgactcctatatgcactg |
| D | H1 | 31 | 2 | 1 ctggcttcaacattaaasattcctatatgcactgggt |
| S | H1 | 32 | 1 | 1 gcttcaacattaaagacsattatatgcactgggtgag |
| Y | H1 | 33 | 2 | 1 tcaacattaaagactccsatatgcactgggtgaggca |
| H | H1 | 35 | 1 | 1 ttaaagactcctatatgsattgggtgaggcaggggcc |
| W | H2 | 50 | 2 | 1 gcctggagtggattggasatattgatcctgagaatgg |
| D | H2 | 52 | 2 | 2 agtggattggatggattsatcctgagaatggtgatac |
| E | H2 | 54 | 2 | 2 ttggatggattgatcctsataatggtgatactgaata |
| N | H2 | 55 | 2 | 2 gatggattgatcctgagsatggtgatactgaatatgc |
| D | H2 | 57 | 2 | 1 ttgatcctgagaatggtsatactgaatatgccccgaa |
| T | H2 | 58 | 1 | 1 atcctgagaatggtgatsatgaatatgccccgaagtt |
| E | H2 | 59 | 2 | 1 ctgagaatggtgatactsattatgccccgaagttcca |
| P | H2 | 62 | 2 | 1 gtgatactgaatatgccsataagttccagggcaaggc |
| K | H2 | 63 | 2 | 3 atactgaatatgccccgsatttccagggcaaggccac |
| Q | H2 | 65 | 2 | 2 aatatgccccgaagttcsatggcaaggccacttttac |
| E | | 98 | 1 | 0 ccgtctattattgtaatsatgggactccgactgggcc |
| G | | 99 | 1 | 0 tctattattgtaatgagsatactccgactgggccgta |
| T | H3 | 100 | 2 | 0 attattgtaatgaggggsatccgactgggccgtacta |
| P | H3 | 101 | 2 | 0 attgtaatgaggggactsatactgggccgtactactt |
| T | H3 | 102 | 2 | 0 gtaatgaggggactccgsatgggccgtactactttga |
| G | H3 | 103 | 2 | 0 atgaggggactccgactsatccgtactactttgacta |
| P | H3 | 104 | 2 | 0 aggggactccgactgggsattactactttgactactg |
| Y | H3 | 106 | 2 | 0 ctccgactgggccgtacsattttgactactggggcca |
| S | L1 | 162 | 2 | 2 taacctgcagtgccagcsatagtgtaagttacatgca |
| S | L1 | 163 | 2 | 1 cctgcagtgccagctcasatgtaagttacatgcactg |
| V | L1 | 164 | 1 | 1 gcagtgccagctcaagtsatagttacatgcactggtt |
| S | L1 | 165 | 2 | 1 gtgccagctcaagtgtasattacatgcactggttcca |
| Y | L1 | 166 | 2 | 1 ccagctcaagtgtaagtsatatgcactggttccagca |
| Y | | 183 | 1 | 0 ctcccaaactcgtgattsatagcacatccaacctggc |
| S | L2 | 184 | 2 | 0 ccaaactcgtgatttatsatacatccaacctggcttc |
| T | L2 | 185 | 1 | 1 aactcgtgatttatagcsattccaacctggcttctgg |
| S | L2 | 186 | 2 | 2 tcgtgatttatagcacasataacctggcttctggagt |
| N | L2 | 187 | 2 | 1 tgatttatagcacatccsatctggcttctggagtccc |
| A | L2 | 189 | 1 | 1 atagcacatccaacctgsattctggagtccctgctcg |
| S | L2 | 190 | 2 | 1 gcacatccaacctggctsatggagtccctgctcgctt |
| R | L3 | 225 | 2 | 2 cttattactgccagcaasattctagttacccactcac |
| S | L3 | 226 | 2 | 2 attactgccagcaaagasatagttacccactcacgt |
| S | L3 | 227 | 1 | 2 actgccagcaaagatctsattacccactcacgttcg |
| Y | L3 | 228 | 1 | 2 gccagcaaagatctagtsatccactcacgttcggtg |
| L | L3 | 230 | 1 | 2 aaagatctagttacccasatacgttcggtgctggcac |

### Sequencing of Variants

Variants were grown overnight with shaking at 37° C. in 5 mL cultures of LA containing 5 ppm of CMP. Miniprep DNA was prepared using a Qiagen kit and the BLA gene within each clone was sequenced using the M13 reverse and nsa154f primers.

| | |
|---|---|
| M13 reverse: | CAGGAAACAGCTATGAC |
| nsa154f: | GGACCACGGTCACCGTCTCCTC |

Screen pH-Dependent Binding

Library NA08 was plated onto agar plates with LA medium containing 5 mg/l chloramphenicol and 0.1 mg/l cefotaxime (Sigma). 552 colonies were transferred into a total of six 96-well plates containing 100 μl/well of LA medium containing 5 mg/l chloramphenicol and 0.1 mg/l cefotaxime. Four wells in each plate were inoculated with TOP10/NA06.6 as a reference. The plates were grown overnight at 37 C. The next day the cultures were used to inoculate fresh plates (production plates) containing 100 ul of the same medium using a transfer stamping tool and glycerol was added to the master plates which were stored at −70 C. The production plates were incubated in a humidified shaker at 37 C for 2 days. 100 ul of BPER (Pierce, Rockford, Ill.) per well was added to the production plates to release protein from the cells. The production plates were diluted 100-fold in PBST (PBS containing 0.125% Tween-20), and BLA activity was measured as above.

Binding to CEA (carcinoembryonic antigen, Biodesign Intl., Saco. Me.) was measured using the following procedure: 96-well plates were coated with 100 ul per well of 5 ug/ml of CEA in 50 mM carbonate buffer pH 9.6 overnight. The plates were washed with PBST and blocked for 1-2 hours with 300 ul of casein (Pierce, Rockford, Ill.). 100 n1 of sample from the production plate diluted 100-1000 fold was added to the CEA coated plate and the plates were incubated for 2 h at room temperature. Subsequently, the plates were washed four times with PBST and 200 ul nitrocefin assay buffer was added, and the BLA activity was measured as described above. CEA binding was measured in 50 mM phosphate buffer pH 6.5 and in a separate experiment in 50 mM phosphate buffer pH 7.4.

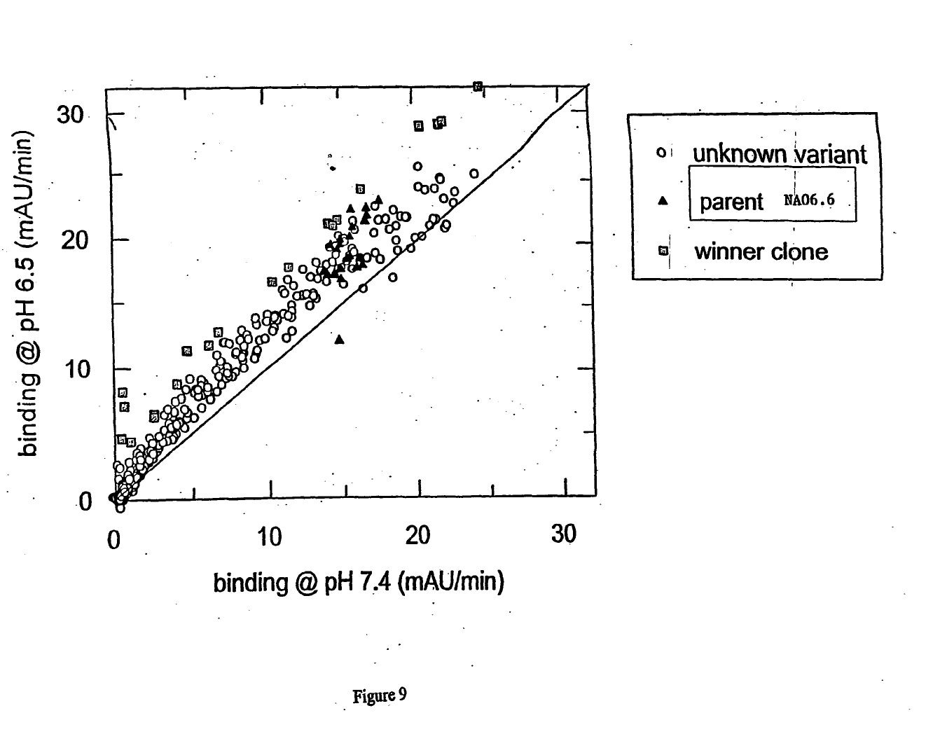

The BLA activity that was determined by the CEA-binding assay at pHs of 6.5 and 7.4, and the total BLA activity found in the lysate plates were compared and variants were identified which showed good binding to CEA at pH 6.5 but significantly weaker binding at pH 6.5. A comparison of the binding at pH6.5 versus pH 7.4 is shown in FIG. 9.

Winners were confirmed by culturing them in 5 ml of LB medium containing 5 mg/l chloramphenicol and 0.1 mg/l cefotaxime (Sigma) for 2 days at 37 C. Subsequently, the cultures were centrifuged and the pellet was suspended in 375 ul of BPER reagent to release the fusion protein. BLA activity was determined as above. One unit of activity was defined as the amount of BLA that leads to an absorbance increase of one mOD per minute. The samples were diluted based on their total content of BLA activity and the CEA-binding assay was performed as described above but adding various sample dilutions to each well.

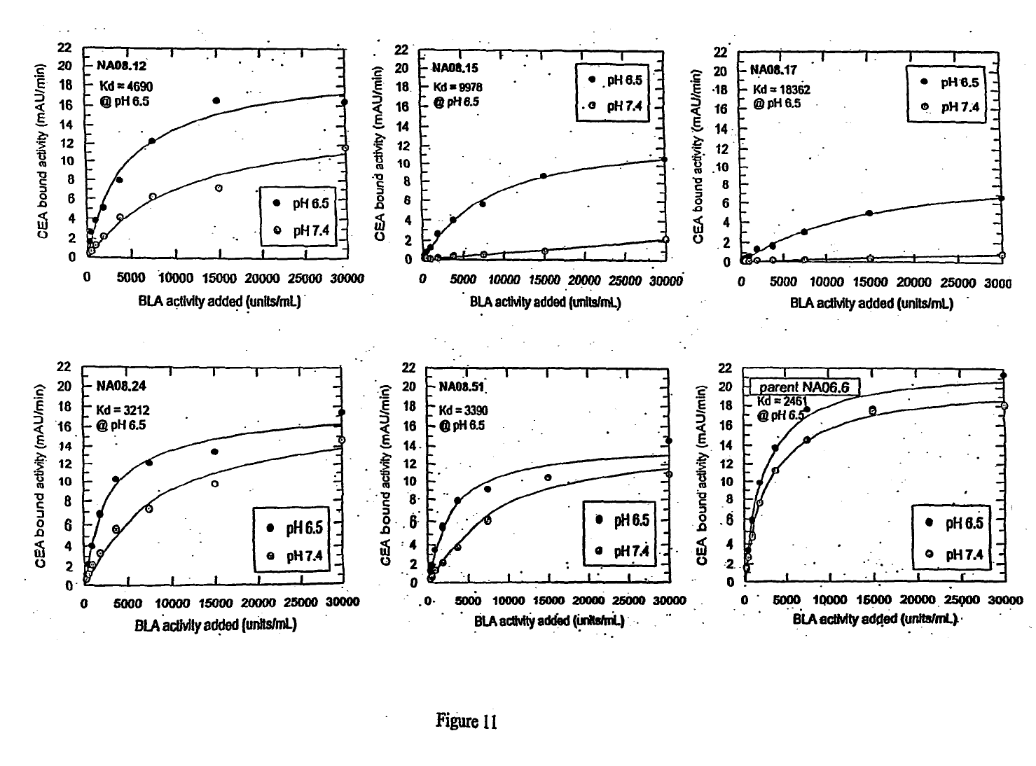

Binding curves for each sample that reflect the affinity of the variants to CEA can be obtained. FIG. 11 shows CEA-binding curves measured at pH 7.4 and pH 6.5 for several variants of interest. All 5 variants show increased pH-dependence of CEA binding. Whereas, the parent NA06.6 binds only slightly better at pH 6.5 compared to pH 7.4, some of the variants show much stronger binding to CEA at pH 6.5 compared to pH 7.4. Variant NA08.15 which shows very weak binding to CEA at pH 7.4 but significant binding at pH 6.5; the variant was designated CAB1.4.

Table 5, below, shows the mutations in variants with the greatest binding improvement

TABLE 5

| Clone | Mutations |
|---|---|
| NA08.1 | W50H, Y166A |
| NA08.3 | S190D, S226D |
| NA08.4 | S190D, T100D |
| NA08.9 | Y166A |
| NA08.12 | T102H, Y166A, S226D |
| NA08.13 | Q65H, S184D, S226D |
| NA08.14 | P101D |
| NA08.15 | S184D, S226D |
| NA08.17 | S184D, W50H |
| NA08.24 | T102D, S226D |
| NA08.45 | T102D, Y166A |
| NA08.51 | P104H, Y166A |
| NA08.64 | Q65D, Y166A |

### Example 3

### Mutagenesis of CAB1.4 Yielding CAB1.6

The codon for position T100 in the CDR3 of the heavy chain of CAB1.4 was subjected to saturation mutagenesis. For site saturation mutagenesis, complimentary oligos:

| | |
|---|---|
| ME 239 F: | ATTATTGTAATGAGGGGNNSCCGACTGGGCCGTACTA |
| ME 239 R: | TAGTACGGCCCAGTCGGSNNCCCCTCATTACAATAAT, |

were designed so a degenerate codon (NNS) would correspond with T100, flanked on either side by 17 base pairs of homology with CAB1.4. The oligo pair was used to carry out a QuickChange (Siratagene) reaction using CAB1.4 DNA as the template according to the manufacturers suggested protocol. After PCR cycling, the reaction mixture was digested with DpnI, and lull was used to transform 50 ul of Invitrogen TOP10 electrocompetent cells. The transformation was plated on LA+5 ppm CMP+0.1 ppm CTX to select for clones that carry the selective marker and still produce active BLA after mutagenesis. Plates were then used to pick clones for screening. After screening, clone ME184.1 (=CAB1.6) that had a T100L mutation (ACT-CTC) was chosen for further optimization.

### Example 4

### Mutagenesis of CAB1.6 Yielding SW149.5

Ten individual site saturation mini-libraries were created for 10 amino acid residues of the H₃CDR (G99, P101-Y109) of CAB1.6 molecule using plasmid pME184.1 as a template with regular QuikChange mutagenesis protocol (Stratagene). After screening for improved affinity, clone pSW129.5 from mini-library SW129 and clone pSW134.1 from mini-library SW134 were isolated. Clone pSW129.5 recruited the T102L mutation from primers ME270F and ME270R, as shown below. Clone pSW134.1 recruited the F107N mutation from primers ME275F and ME275R, as shown below. Clone pSW129.5 was used as a template for further mutagenesis and to isolate clone pSW149.5 as described below.

Several mutations at positions P104 and Y105 were also identified in this screen. To combine those mutations as well as the F107N mutation of clone pSW134.1 into pSW129.5 backbone, a limited randomized library was created with primers SW133F and SW133R using pSW129.5 as a template. Subsequently, clone pSW149.5 was selected based on improved expression and affinity.

The following primers were used, as described above:

ME270F
GTAATGAGGGGCTGCCGNNSGGGCCGTACTACTTTGA
ME270R
TCAAAGTAGTACGGCCCSNNCGGCAGCCCCTCATTAC
ME275F
CGACTGGGCCGTACTACNNSGACTACTGGGGCCAAGG
ME275R
CCTTGGCCCCAGTAGTCSNNGTAGTACGGCCCAGTCG
SW133F
GAGGGGCTCCCGCTCGGGRVCNTTTACAACGACTACTGGGGCCAAGG
SW133R
CCTTGGCCCCAGTAGTCGTTGTAAANGBYCCCGAGCGGGAGCCCCTC

### Example 5

### Mutagenesis of SW149.5 Yielding CAB1.7

Limited randomization of several amino acid residues of H2, L1 and L2 CDRs was achieved employing several degenerate primers. Residues targeted for limited randomization were: D57, T58, P62 and Q65 in the H2 CDR; S163, S165 and S166 in the L1 CDR and S186 and S190 in the L2 CDR. Screening of these variants allowed identification of positions in the protein likely to further improve its affinity for CEA. Library SW155 was created using primers SW134FP, SW135FP, SW136FP, SW137FP and SW138FP using the QuikChange multisite mutagenesis kit (Stratagene) as recommended by the manufacturer. The resulting library was screened and the best variant, clone pSW155.17 was selected as it showed significantly improved binding to CEA; the clone was designated CAB1.7.

The following primers were used to generate library SW155:

SW134FP
[Phosp]CTTCTGGCTTCAACATTACCGACTCCTATATGCACTG
SW135FP
[Phosp]GCCTGGAGTGGATTGGATTTATTGATCCTGAGAATG
SW136FP
[Phosp]GATCCTGAGAATGGTSWTRCTGAATATGCCCBGAAGTTCRNCG
GCAAGGCCACTTTTAC
SW137FP
[Phosp]CTGCAGTGCCAGCFCADCTGTAYMTDCCATGCACTGGTTCCAGC
SW138FP
[Phosp]CGTGATTTATGATACARVCAACCTGGCTRSTGGAGTCCCTGCT
CGCTTC

### Example 6

### Generation of CAB1.6i and CAB1.7i

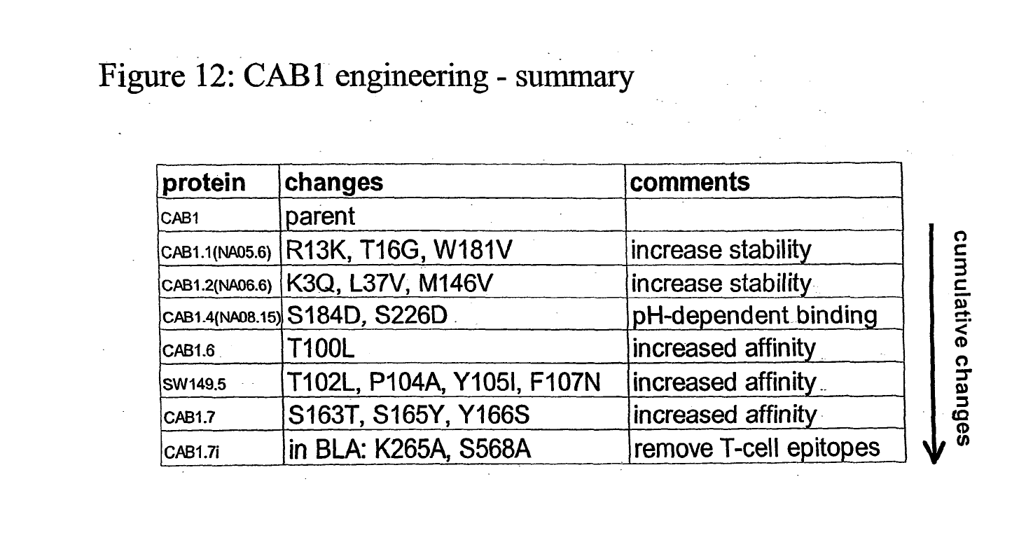

FIG. 12 shows the development of CAB1.6i and CAB1.7i and demonstrates the incorporation of mutations in the process.

In order to compare the target-binding properties of various CAB1 variants, we grew 5 ml cultures of TOP10F′ containing the corresponding expression plasmids, as provided above for 3 days at 25 C in LB medium containing 5 mg/l chloramphenicol. The cultures were centrifuged, and the resulting supernatant was discarded. The cell pellets were resuspended in 500 μL of B-PER reagent. This was incubated for 30 minutes. Lactamase concentration in each sample was determined using nitrocephin as substrate, as provided above.

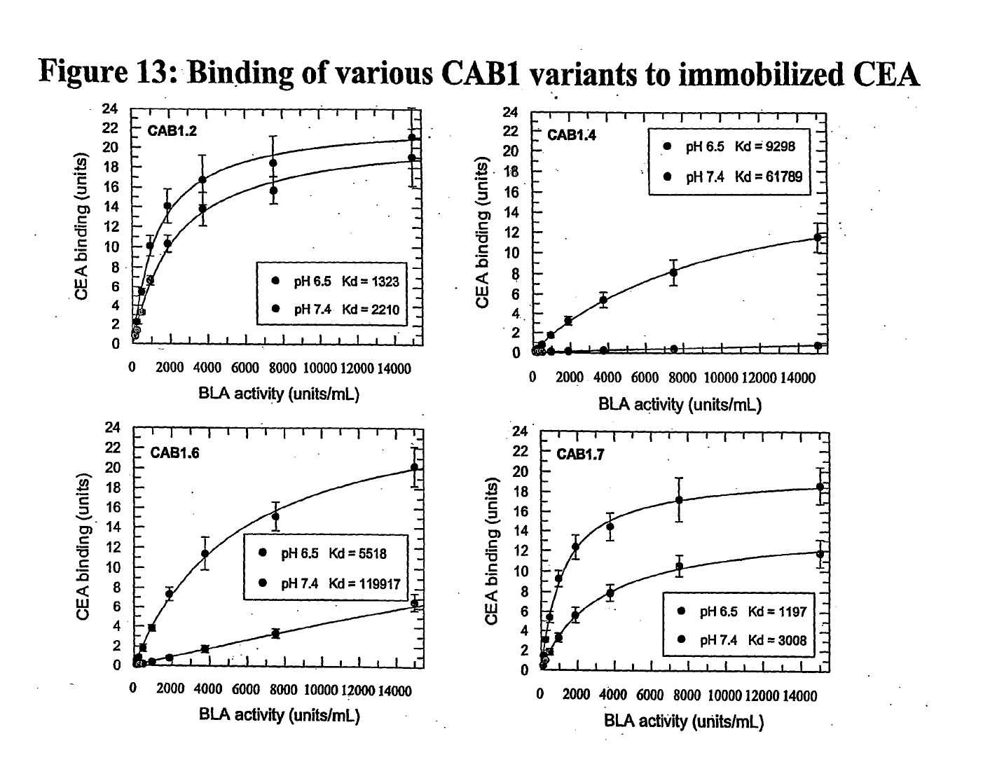

Binding of the samples to microtiter plates coated with CEA was studied in 50 mM phosphate buffer at pH 6.5 and pH 7.4, as provided above. Binding curves are shown in FIG. 13.

In a similar experiment, binding of variants to LS174T cells was measured. LS174T cells were inoculated in 96 well polystyrene plate at 1×10⁵ cells/well in a medium containing 70% DMEM, 30% F12, non-essential amino acids, L-Glut, and Sodium Pyruvate (all from Mediatech). The plate was incubated at 37° C. in a humidified CO2 incubator for 20 hours. The cells were then fixed with 4% formaldehyde in PBS (Polysciences, Warrington, Pa.). The plate was washed with PBST, and 1 mg/ml NaBH4 (Sigma) was added into each well to quench any reactive group. Then the plate was washed again with PBST. Binding of CAB molecules was continued the same way as binding to CEA antigen.

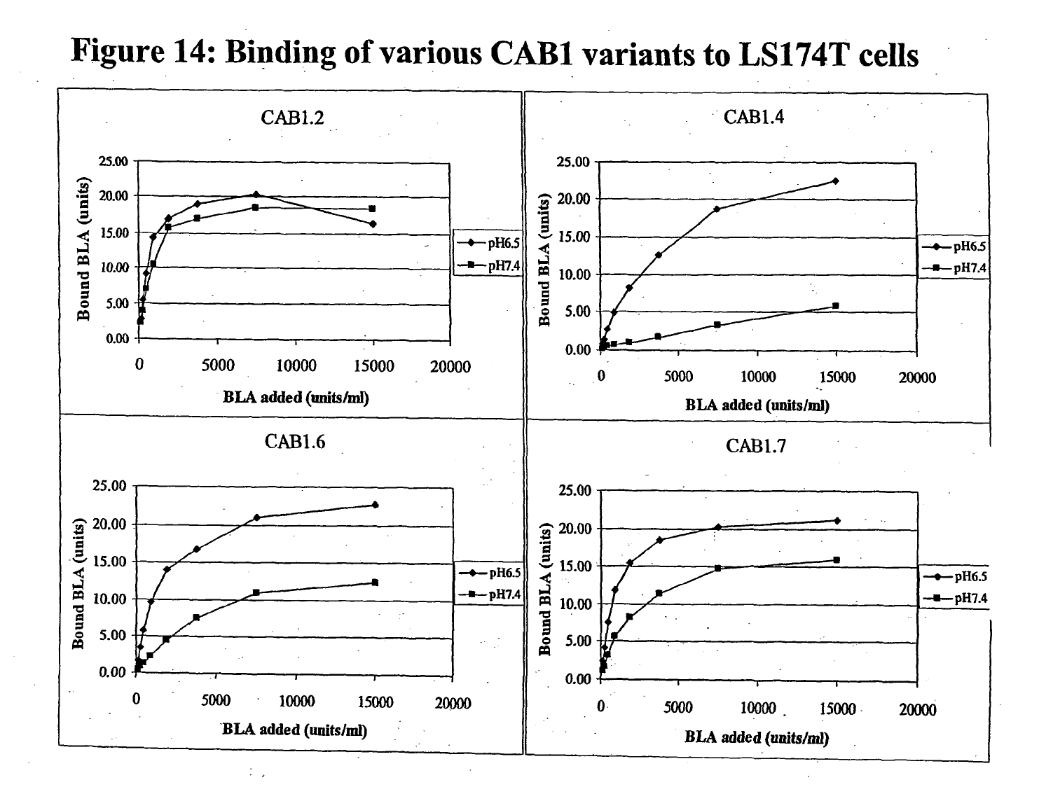

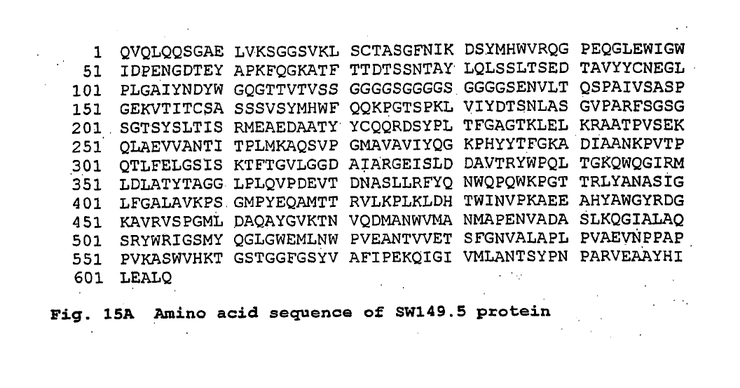

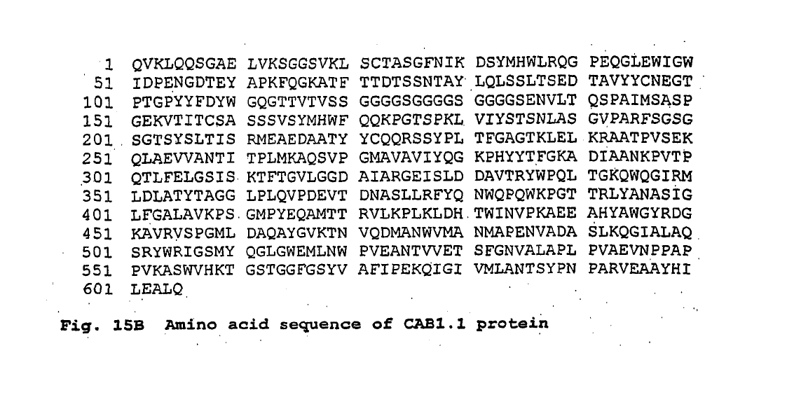

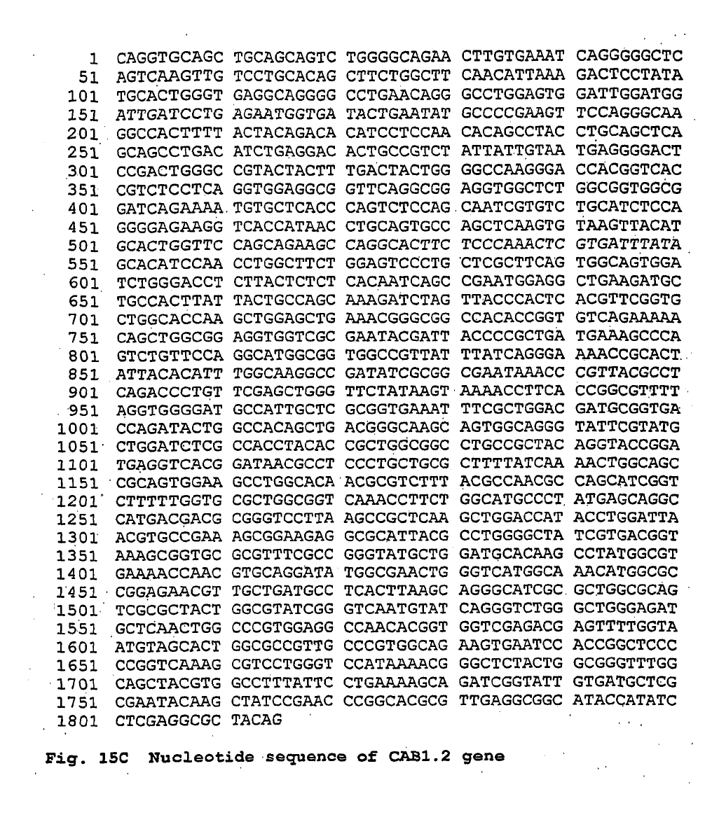

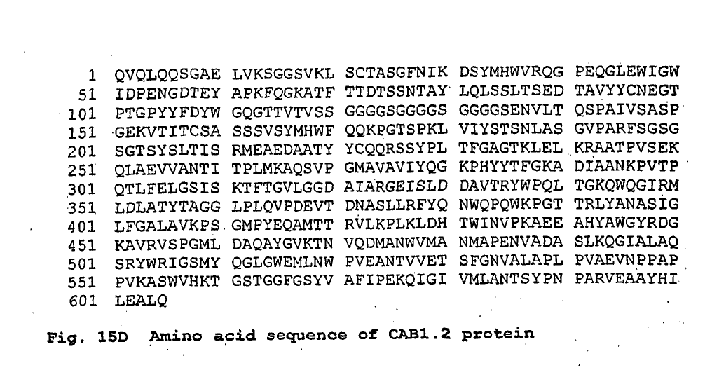

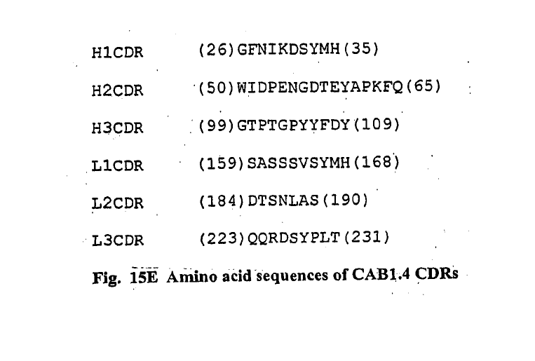

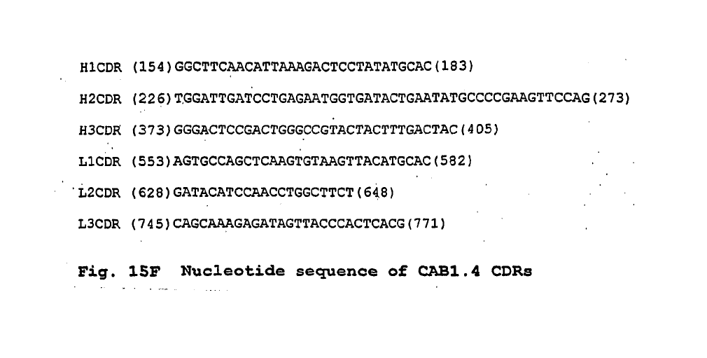

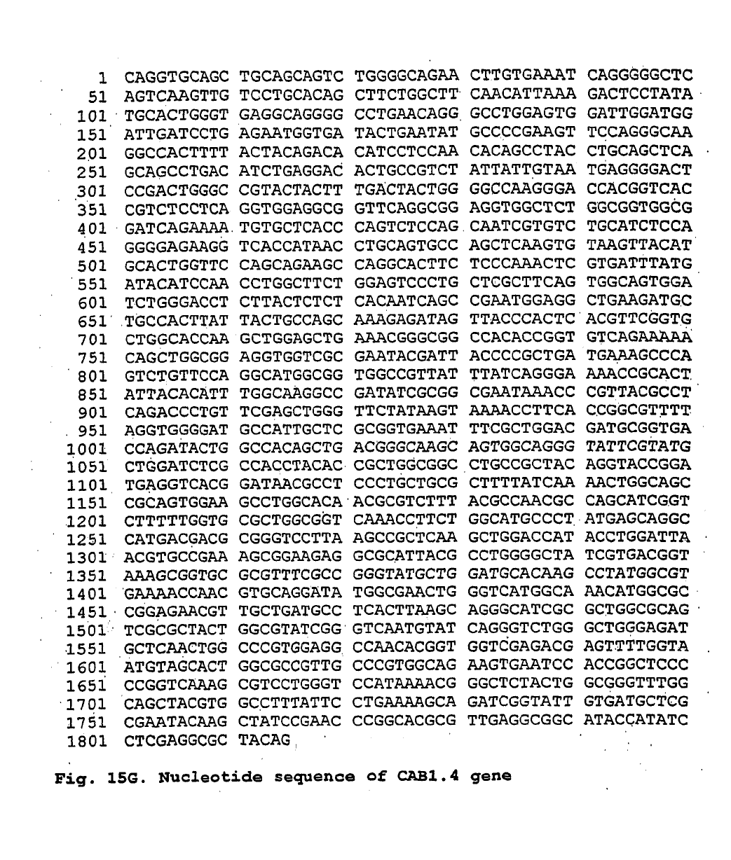

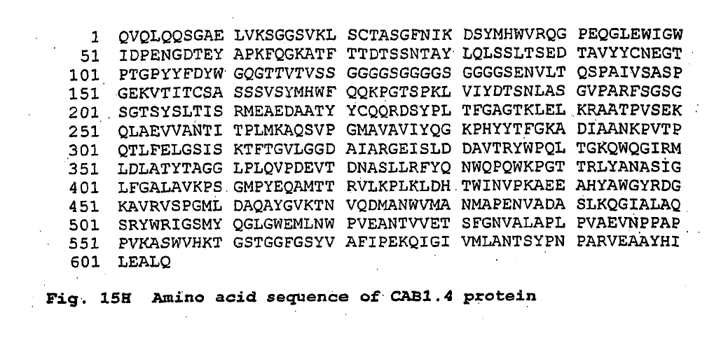

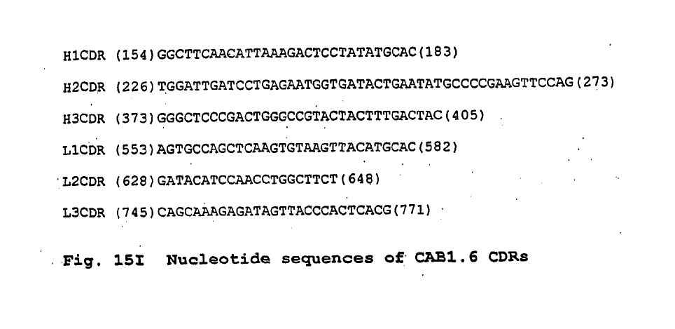

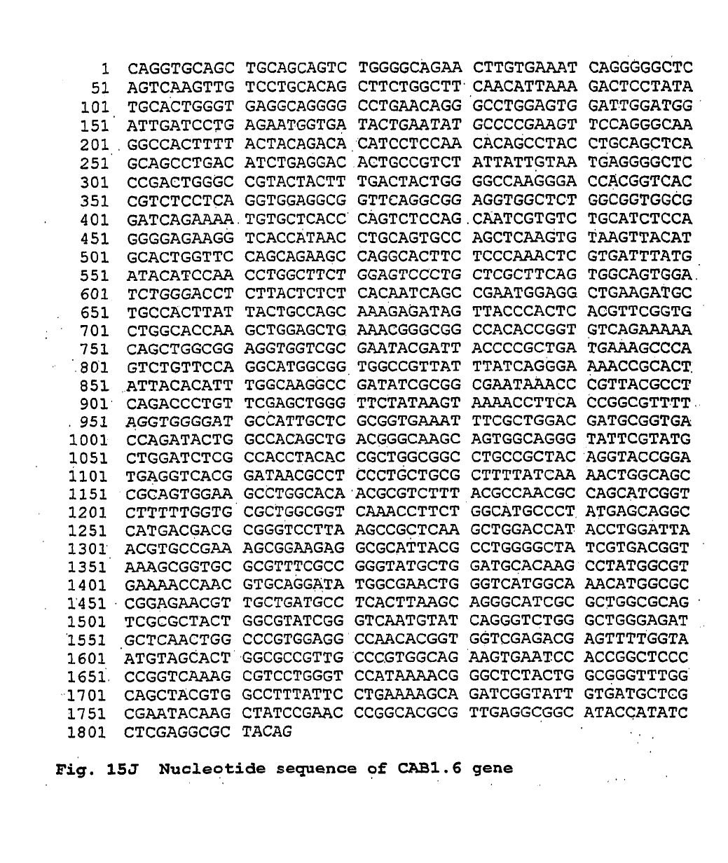

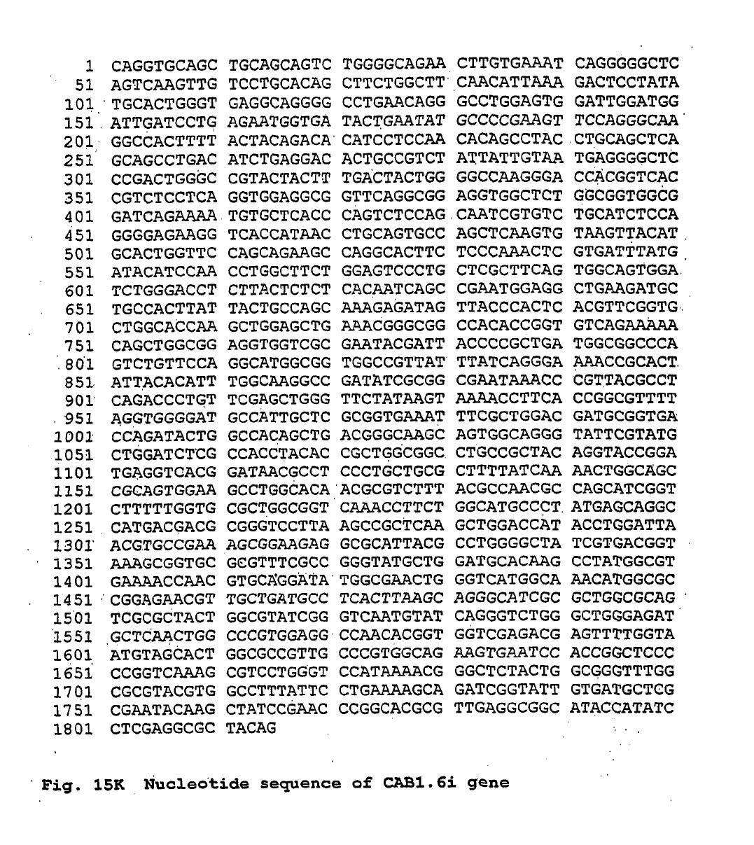

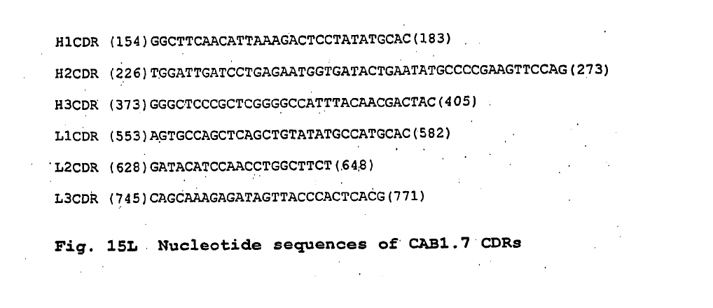

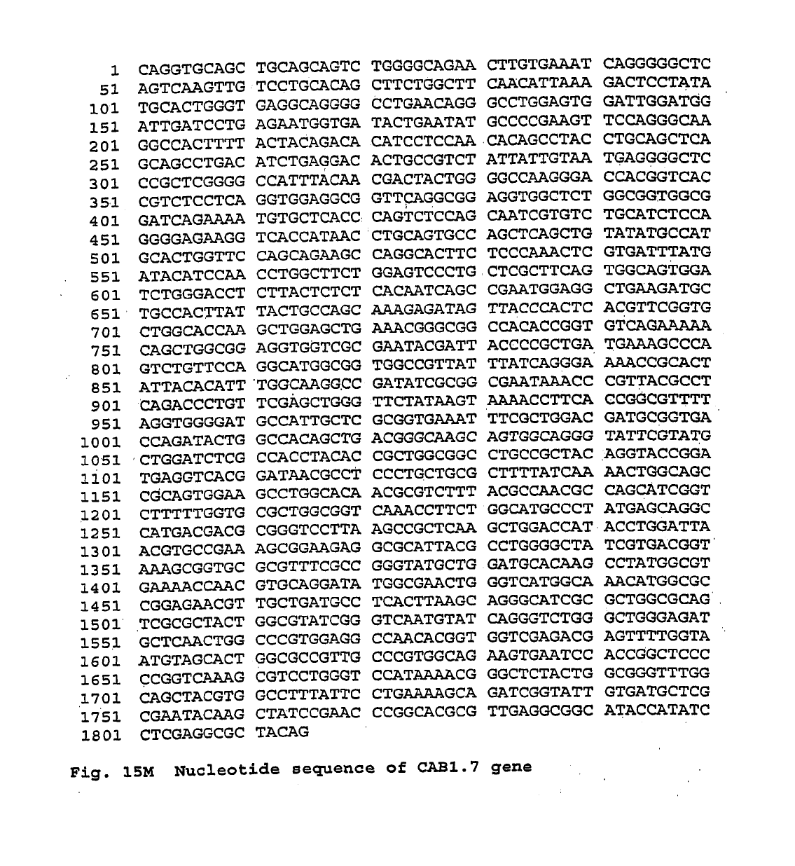

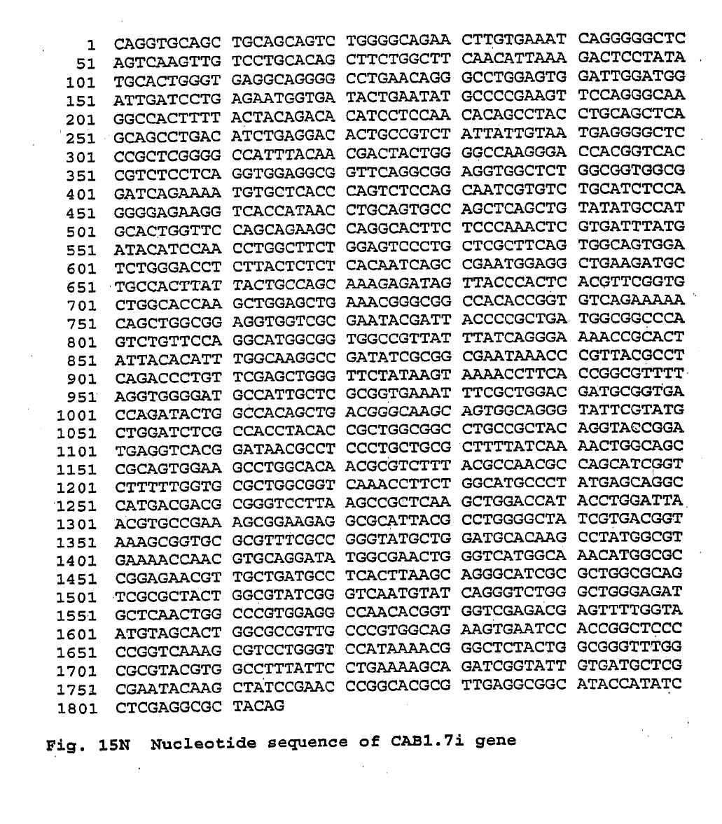

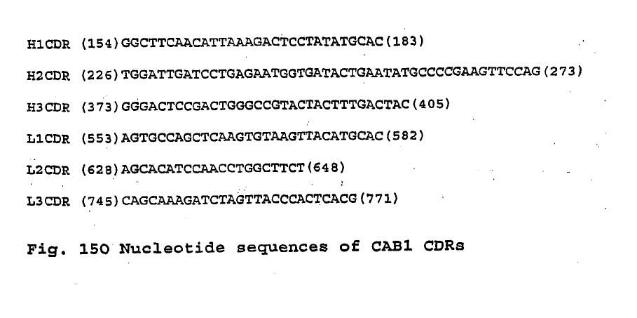

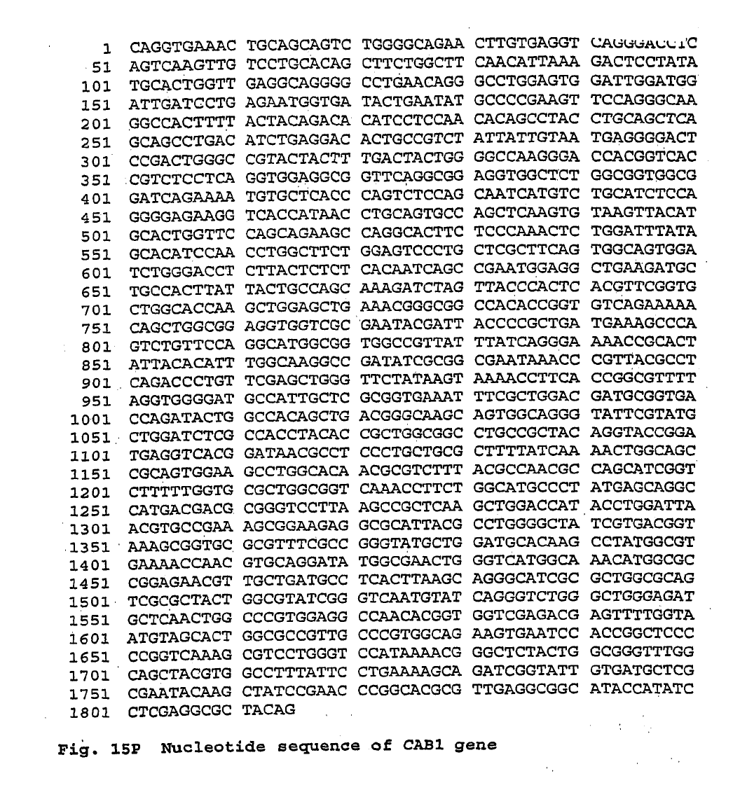

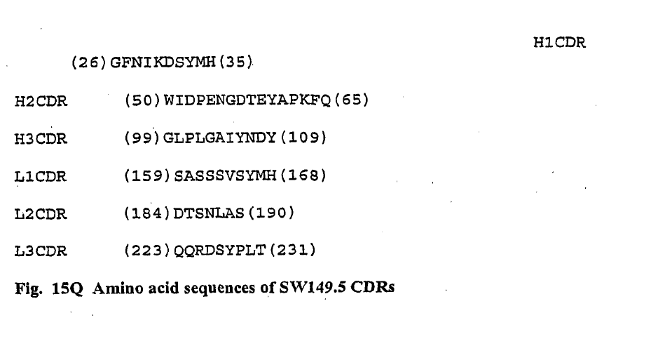

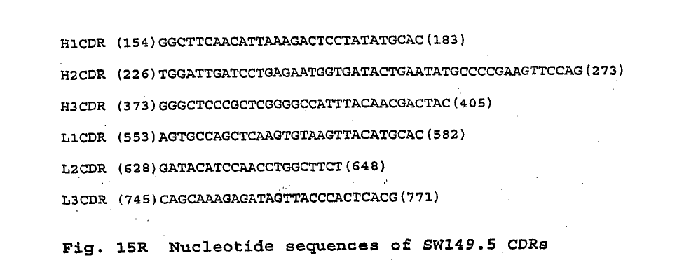

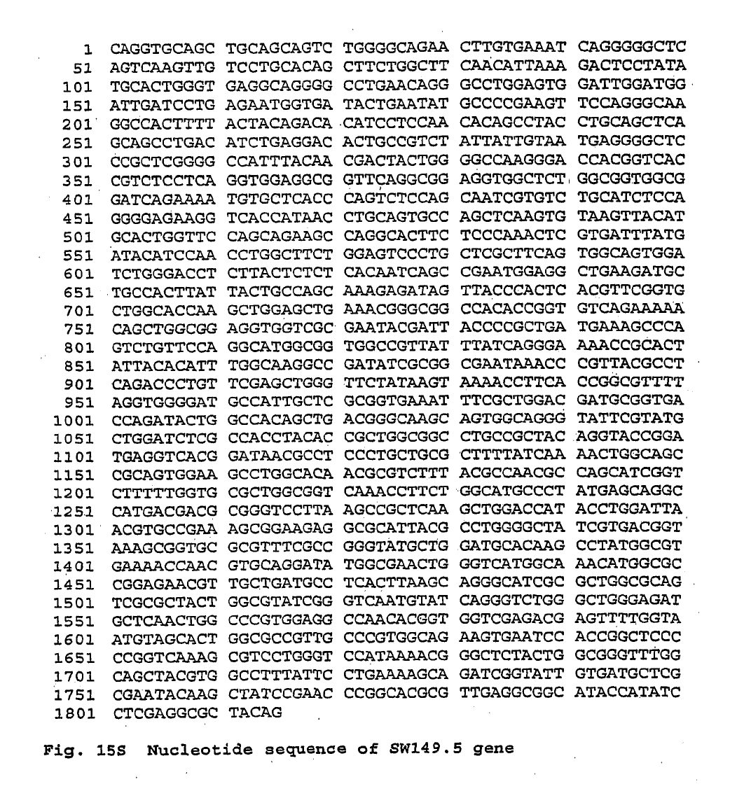

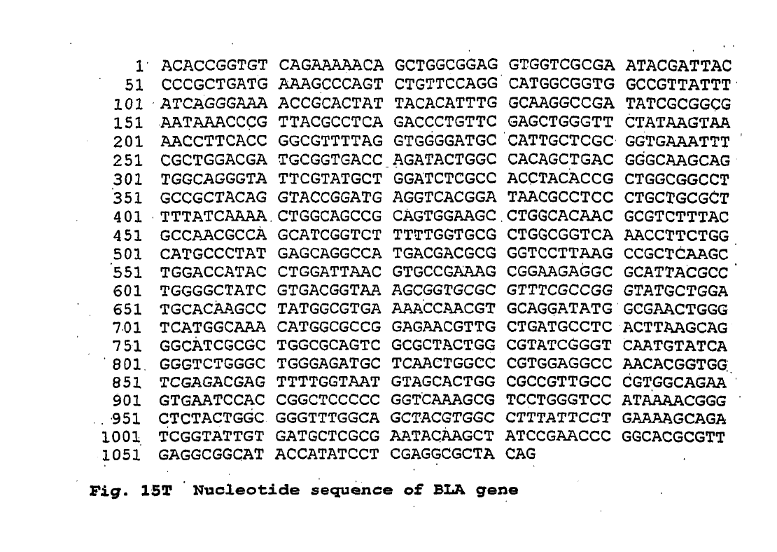

FIG. 14 shows binding curves for CAB1.2, CAB1.4, CAB1.6, and CAB1.6 to LS174T cells. CAB1.7 has a binding affinity at pH6.5 that closely resembles the binding curve of CAB1.2 at the same pH. In contrast, the binding curves at pH 7.4 show marked to differences. At pH 7.4, CAB1.7 shows significantly weaker binding to tumor cells as compared to CAB1.2. Surprisingly, CAB1.7 binding curves reach saturation levels that are also pH-dependent. This suggests, that at saturation, more molecules of CAB1.7 can bind to tumor cells at pH6.5 as compared to pH7.4.

### Example 7

### Epitope Removal of BLA

The i-mune assay was performed on the sequence for beta-lactamase as described (U.S. patent application Ser. No. 09/060,872, filed Apr. 15, 1998). Human population-based identification of CD4+ T cell peptide epitope determinants. (Journal of Immunological Methods, 281:95-108). Sixty-nine community donor peripheral blood cell samples were used. Four CD4+ T cell epitopes were identified. For each epitope peptide sequence, critical residue testing was performed. Critical residue testing included both an alanine scan of the peptide sequences, as well as specific amino acid modifications guided by functional and structural constraints. Peptide epitope sequences that reduced the level of proliferation to background levels were chosen and incorporated into a DNA construct of the beta-lactamase enzyme sequence. Modified enzyme protein variants were expressed and purified, then tested for their ability to induce cellular proliferation using human peripheral blood cells in vitro. The variant that induced the lowest level of cellular proliferation in vitro was selected for inclusion in CAB1.6 and CAB1.7.

### Example 8

### Construction of CAB1.6i and CAB1.7i

BLA genes in plasmids pME184.1 (CAB1.6) and pSW155.17 (CAB1.7) were mutated in order to introduce the de-immunized BLA (=BLAi) gene containing epitope-removing K265A and S568A mutations as described below. Using primers HR016F and HR017F with the QuikChange Multisite mutagenesis kit (Stratagene) as recommended by the manufacturer, the two mutations were incorporated into plasmid pME184.1 (CAB1.6) resulting in plasmid pSW175.3 (CAB1.6i). For construction of plasmid pSW169.3 (CAB1.7i), a 0.9-kb NruI fragment of the BLA gene in plasmid pSW155.17 was exchanged with another 0.9-kb NruI fragment from plasmid pCD1.1 which contains both mutations.

The following primers were used:

HR016F
[Phosp]GATTACCCCGCTGATGGCGGCCCAGTCTGTTCCAG
HR017F
[Phosp]CTACTGGCGGGTTTGGCGCGTACGTGGCCTTTATTCCTG

### Example 9

Pharmacokinetics and Tissue Distribution of CAB1.11i and CAB1.13i in T1918 Tumor Bearing Athymic Mice

Study design is outlined in Table 6. Fifty female mice, 18-22 g, approximately 6-8 weeks, from Taconic Labs, were implanted with tumor derived T1918 cells by subcutaneous injection suspended in DMEM media at 5×10⁷ cells/mL. Animals were anesthetized by isoflurane inhalation, and cells were implanted by subcutaneous injection of 100 uL cell suspension (approximately 5×10⁶ cells/mouse).

TABLE 6
Study Design

| | | | | | Dose | | |
|---|---|---|---|---|---|---|---|
| | | Test | Dose | Conc. | Volume | Timepoints | |
| Group | N/Sex | Article | (mg/kg) | (mg/mL) | (mL/kg) | (hours) | Tissues |
| 1 | 3/F | None | 0 | — | — | 0 | Plasma, |
| 2 | 12/F | CAB1.11i | 0.25 | 0.05 | 5 | 6, 12, 24 | tumor, |
| 3 | 12/F | CAB1.11i | 1 | 0.2 | 5 | and 48 | liver and |
| 4 | 12/F | CAB1.13i | 1 | 0.2 | 5 | | kidney |

After tumor implantation, animals were observed daily at minimum and moribund or distressed animals were euthanized. Tumors were measured twice weekly.

When tumors reached approximately ≧250 mm³, 39 animals were selected based on tumor size and growth rate and randomized into 4 groups. Three mice in Group 1 were administered nothing, and twelve animals in each of Groups 2-4 were administered a single IV bolus injection of CAB 1.11i or CAB 1.13i (1 mg/kg). CAB 1.11i and CAB1.13i were formulated in 0.05 mg/mL and/or 0.2 mg/mL, respectively, using PBS and injected within 60 minutes. Injections of approximately 100 uL/mouse were administered via the tail vein.

Mice were weighed on the day of dosing, and doses were based on the average weight of all animals. Mice were warmed with a heat lamp and heating pad and placed in is a restrainer. The tail was wiped with 70% alcohol and doses were administered by bolus intravenous injection via the tail vein. At 6, 12, 24 and 48 hours post dose administration, 3 animals from each group were anesthetized with isoflourane, and blood was collected by cardiac puncture into EDTA. The blood samples were centrifuged within 20 minutes of collection and the plasma fraction was collected and frozen in a −70° C. freezer.

Three animals from each group were euthanized at 6, 12, 24 and 48 hours post CAB administration for collection of plasma, tumor, liver and kidney, and analyzed for CAB concentration. The livers, kidneys and tumors from all animals was collected, rinsed, blotted, weighed and snap frozen in liquid nitrogen. Blood and tissue samples from the control group were collected at baseline only. The tissue samples were homogenized on ice in PBS with 15 ug/mL aprotinin (2 mL buffer:gram tissue). The homogenate was mixed with B-PER (1:1) and centrifuged. CAB concentrations in the tissue supernatant and plasma samples were determined by measuring BLA activity using a nitrocefin assay, as provided above.

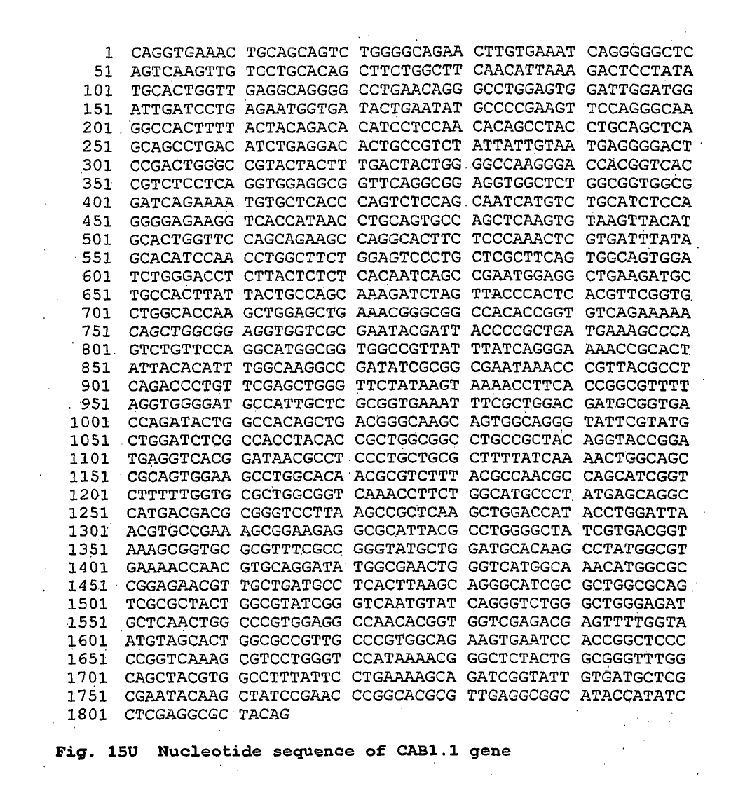

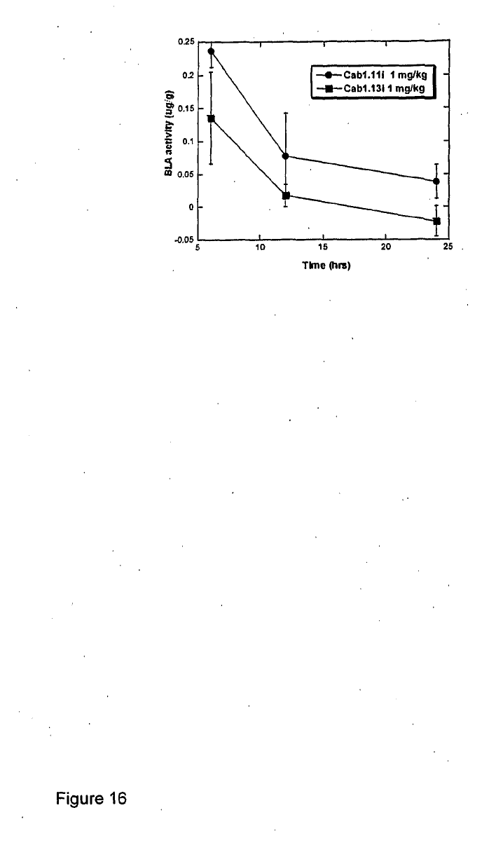

The results are shown in FIG. 16.

### Example 10

### Anti-Tumor Activity of C-Mel or Glutaryl-C-Mel when Administered 24 Hrs after CAB 1.2 in LS174T SCID Model

Female CB17-SCID mice (7-9 weeks, Taconic Labs) were challenged subcutaneously with 2×10⁶ LS174T cells suspended in serum free DMEM in a volume of 100 microliters (Medimmune ACUC protocol # ACF 037). When mean subcutaneous (SC) tumor volumes were approximately 100-150 mm³, animals were randomly distributed into treatment groups Animals without detectable tumors or excessively large tumors (volume >300 mm³) were excluded. Animals were dosed with CAB1.2 and/or prodrug according to study design (Table 7).

TABLE 7
Study Design

| | | | CAB1.2 | | | TOA | |
|---|---|---|---|---|---|---|---|
| Group | N/Sex | ROA | Dose | Prodrug | Dose | Prodrug | Observations |
| 1 | 10F | — | — | Untreated | — | — | Body weight: |
| 2 | 10F | IV | — | Glutaryl-C-Mel | 150 mg/kg | 24 hr | weekly |
| 4 | 10F | IV | 1 mg/kg | C-Mel | 150 mg/kg | 24 hr | Tumor |
| | | | Lot No. | | | | measurements |
| 5 | 10F | IV | 1 mg/kg | Glutaryl-C-Mel | 150 mg/kg | 24 hr | 2x/week |
| | | | Lot No. | | | | Cage side |
| 6 | 10F | IV | 1 mg/kg | Glutaryl-C-Mel | 75 mg/kg | 24 hr | observations |
| | | | | | | | Daily except |
| | | | | | | | weekend |

1. Preparation of Dosing Solutions

C-Mel is formulated as follows: A100 mg/mL stock solution of C-Mel in DMSO stored frozen at −70° C. is thawed, immediately added to 1.0 M Na bicarbonate at a C-Mel: 1.0 M NaHCO₃ ratio (V/V) of 3.5:1, vortexed, diluted in 5% aqueous sucrose solution to a final concentration of 15 mg/ml, filter sterilized through a 0.2 micron filter unit and placed in an ice bath until use.

Glutaryl-C-Mel is formulated as follows: the drug is weighed and dissolved in 3.0 eq of 1.0 M NaHC0₃. The solution is mixed well by vortex and diluted with 5% aqueous sucrose solution to 30 mg/mL final concentration. The solution is further diluted with PBS to 20 mg/mL and kept on ice packs until administered.

Tumor-bearing animals received 100 microliters of CAB1.2 formulated in PBS at a dose of 1 mg/kg as a single IV bolus injection via the tail vein. 24 hours after CAB1.2 administration, animals were be administered a single IV bolus of C-Mel or Glutaryl-C-Mel at 75 mg/kg or 150 mg/kg according to study design.

Toxicity was monitored by daily observations and once weekly weight determinations. Tumor measurements were taken twice weekly. Treatment groups whose average tumor volume exceeds 2000 mm³ were euthanized, and individual animals whose tumor was excessively large and/or necrotic were euthanized. Treatment groups were euthanized if fewer than 6 animals remained in the study, except to monitor individual animals that achieve a complete response for tumor regrowth. Data was collected, mean tumor volumes for all treatment groups determined±SEM, and plotted for analysis (days post tumor challenge vs tumor volume).

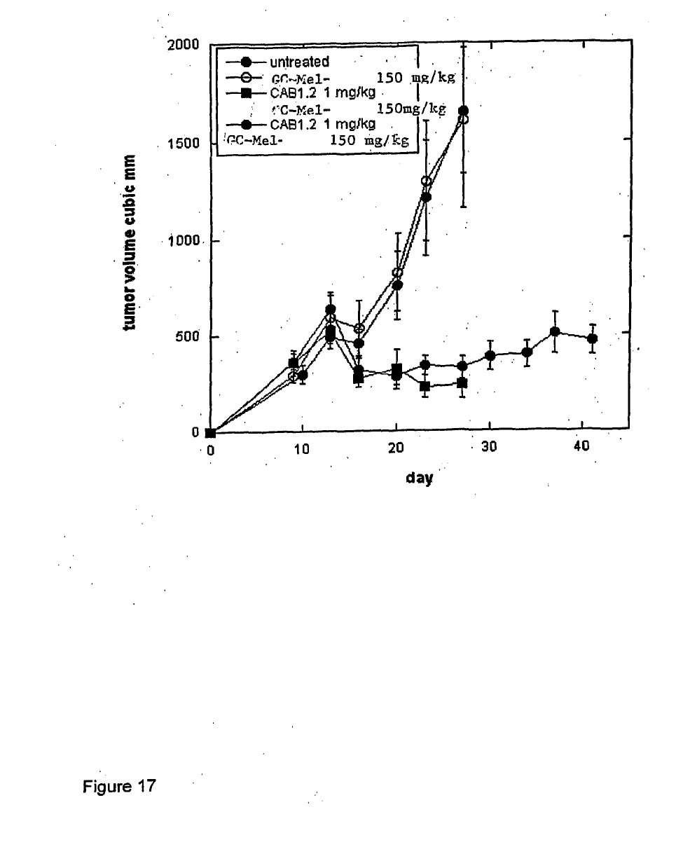

Tumor response is shown in FIG. 17, where the x-axis is time in days, and the y-axis is tumor volume measured in mm3.

FIG. 18 shows toxicity-survival. The x-axis shows time in days, and the y-axis shows the integer number of living mice.

FIG. 19 shows toxicity-body weight. The x-axis shows time in days, and the y-axis shows body weight percentage.

### Example 11

### Glutaryl-C-Mel-L-Phe-NH2 Efficacy and Toxicity Following CAB1.2 Administration in T-LS-174-T Tumor Bearing Athymic Mice

Study design is outlined in Table 8. Forty female Ncr mice, 18-22 g, from Taconic Labs, were implanted with tumor derived TLS174T cells by subcutaneous injection suspended in DMEM at 2×10⁷ cells/mL. Animals were anesthetized by isoflurane inhalation, and cells were implanted by subcutaneous injection of 100 uL cell suspension (approximately 2×10⁶ cells/mouse).

TABLE 8
Study Design

| | | | glutaryl- | Post-Cab | |
|---|---|---|---|---|---|
| | | | C-Mel-L- | Time of | |
| | | CAB1.2i | Phe-NH2 | Adminis- | |
| | | Dose | Dose | tration | |
| Group | N/Sex | (mg/kg) | (mg/kg) | (hours) | Observations |
| 1 | 5/F | 0 | 0 | NA | Body weight: |
| 2 | 5/F | 1 | 50 | 24 | Days 1, ~4 and 8 |
| 3 | 5/F | 1 | 100 | 24 | Cage side obser- |
| 4 | 5/F | 1 | 100 | 24 and 48 | vations: daily |
| | | | | | Tumor |
| | | | | | Measurements: |
| | | | | | twice weekly |

20

A stock solution was prepared by dissolving glutaryl-C-Mel-L-Phe-NH2 in DMSO to a concentration of 100 mg/mL in a sterile polystyrene tube with screw cap. The stock solution was diluted with sterile filtered NaHCO3 (1M) to achieve a 3:1 molar ratio of bicarbonate to drug and mixed well by using a vortex mixer. The solution was diluted to 10 mg/mL with 5% (w/v) sterile sucrose, resulting in a 10% DMSO concentration. The stock solution was prepared within 24 hours of dilution, diluted material and administered within 60 minutes of preparation.

When tumors reached approximately ≧250 mm³, 20 animals were selected based on tumor size and growth rate and assigned into 4 groups. Five mice each were administered nothing or CAB1.2i (1 mg/kg) followed by a single dose of glutaryl-C-Mel-L-Phe-NH2 (50 or 100 mg/kg) 24 hours after CAB administration or two doses of glutaryl-C-Mel-L-Phe-NH2 (100 mg/kg) at 24 and 48 hours post CAB administration. The CAB was formulated to 0.2 mg/mL and glutaryl-C-Mel-L-Phe-NH2 formulated to 10 mg/mL, as provided in Table 9.

TABLE 9
Test Article Concentrations and Dose Volumes

| | Formulated | |
|---|---|---|
| | Concentration | Dose Volume |
| Test Article | (mg/mL) | (mg/kg) |
| CAB1.2i | 0.2 | 5 |
| glutaryl-C- | 10 | 10 |
| Mel-L-Phe- | | |
| NH2 | | |

All test articles were injected within 60 minutes of dilution and formulation. Injections of approximately 100 uL/mouse (CAB1.2i) or 200 uL/mouse (glutaryl-C-Mel-L-Phe-NH2) were administered via the tail vein.

When tumors reached ≧250 mm³, animals were assigned to groups. Mice were weighed on the day of dosing (Day 1), and doses were based on the average weight of all animals. Mice were warmed with a heat lamp and heating pad and placed in a restrainer. The tail was wiped with 70% alcohol and doses were administered by bolus intravenous injection via the tail vein.

Subsequent body weights were determined on Day 8 and on an intermediate day depending on scheduling (Day 4 or 5). Animals were observed cage side for signs and symptoms of toxicity. Moribund or distressed mice were sacrificed and underwent necropsy. A necropsy was performed within 2 hours of discovery of any animals that were found dead. On Day 8, all animals were euthanized by CO₂ inhalation and underwent necropsy. Kidneys and tumors from all animals, as well as any abnormal tissues or organs, were formalin fixed for histopathology at the time of necropsy.

Tumors were measured twice weekly for 45 days. Treatment groups whose average tumor volume exceeded 2000 mm³ were euthanized, and individual animals whose tumor was excessively large and/or necrotic were euthanized. On Day 45, all remaining animals were euthanized by CO₂ inhalation.

### Example 12

### Anti-Tumor Activity of CAB 1.2 in LS184T Human Colorectal Model in SCID Mice

Female CB17-SCID mice (8-10 weeks, Taconic Labs) were challenged subcutaneously (SC) with 2×10⁶ LS174T cells suspended in serum free DMEM in a volume of 100 microliters (Medimmune ACUC protocol # ACF 037). When mean tumor volumes were approximately 100-150 mm³, animals were randomly distributed into treatment groups. Animals without detectable tumors or excessively large tumors (volume >300 mm³) were excluded. In this study, CAB1.2 and a p97-specific ADEPT construct (P97ADEPT) that does not bind significantly to LS174T cells were administered on days 8, 14, and 21 post-tumor cell inoculation, followed by C-Mel dosing.

Tumor-bearing animals received CAB1.2 in PBS intravenously (IV) at doses of 1 or 2.5 mg/kg in an injection volume of 100 microliters in PBS. C-Mel stored at −70° C. as a 100 mg/ml frozen solution in DMSO) was formulated fresh (15 mg/ml, in a 5:4 ratio of 0.1M Na bicarbonate:PBS) and administered at a dose of 150 mg/kg on an average weight basis, IV bolus, via the tail vein in an injection volume of 200 microliters to all treatment groups receiving prodrug. Melphalan was formulated fresh (2 mg/ml in 20% DMSO/PBS) and administered intraperitoneally in an injection volume of 100 microliters. Treatment groups are listed in the Table 10, below. Briefly, animals administered CAB1.2 at 2.5 mg/kg were administered C-Mel 18 or 36 hours after CAB1.2 treatment. Animals administered CAB1.2 at 1 mg/kg were administered C-Mel 24 hours after CAB1.2 treatment. Control groups were as follows: untreated, 2.5 mg/kg CAB1.2 alone, 10 mg/kg melphalan, C-Mel alone, 2.5 mg/kg P97ADEPT followed by C-Mel 18 hours later, 1.5 mg/kg Beta-lactamase (BLA, 1.5 mg/kg, which was equimolar to the 2.5 mg/kg CAB1.2 BLA concentrations used), followed by C-Mel 18 hours later. Treatments were repeated once weekly for 3 cycles. Tumor measurements (in millimeters) were taken twice weekly; the investigator measuring the tumors was blinded to the treatment groups.

TABLE 10

| | | CAB1.2 dose or | C-Mel dose | C-Mel |
|---|---|---|---|---|
| | | P97ADEPT dose | or melphalan | administration |
| Group | N/Sex | or BLA dose | dose | time |
| 1 | 10 F | 2.5 mg/kg | 150 mg/kg | 18 h |
| 2 | 10 F | 2.5 mg/kg | 150 mg/kg | 36 h |
| 3 | 10 F | 1 mg/kg | 150 mg/kg | 24 h |
| 4 | 10 F | Untreated control | — | — |
| 5 | 10 F | 2.5 mg/kg | — | — |
| 6 | 10 F | — | 150 mg/kg | —^{a} |
| 7 | 10 F | — | 10 mg/kg | — |
| | | | melphalan | |
| 8 | 10 F | 2.5 mg/kg | 150 mg/kg | 18 h |
| | | P97ADEPT | | |
| 9 | 10 F | 1.5 mg/kg | 150 mg/kg | 18 h |
| | | BLA | | |

Animals were monitored daily for general appearance, animal weights taken once weekly and tumor measurements taken twice weekly. Data was collected and mean tumor volumes for all treatment groups determined±SEM, and plotted for analysis (days post tumor challenge vs tumor volume).

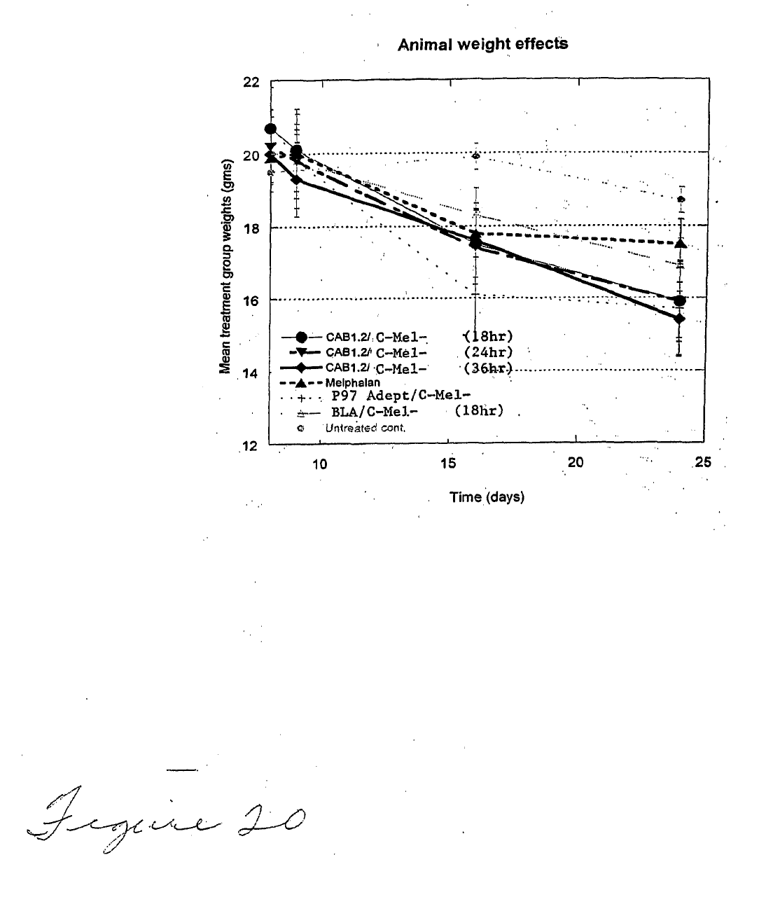

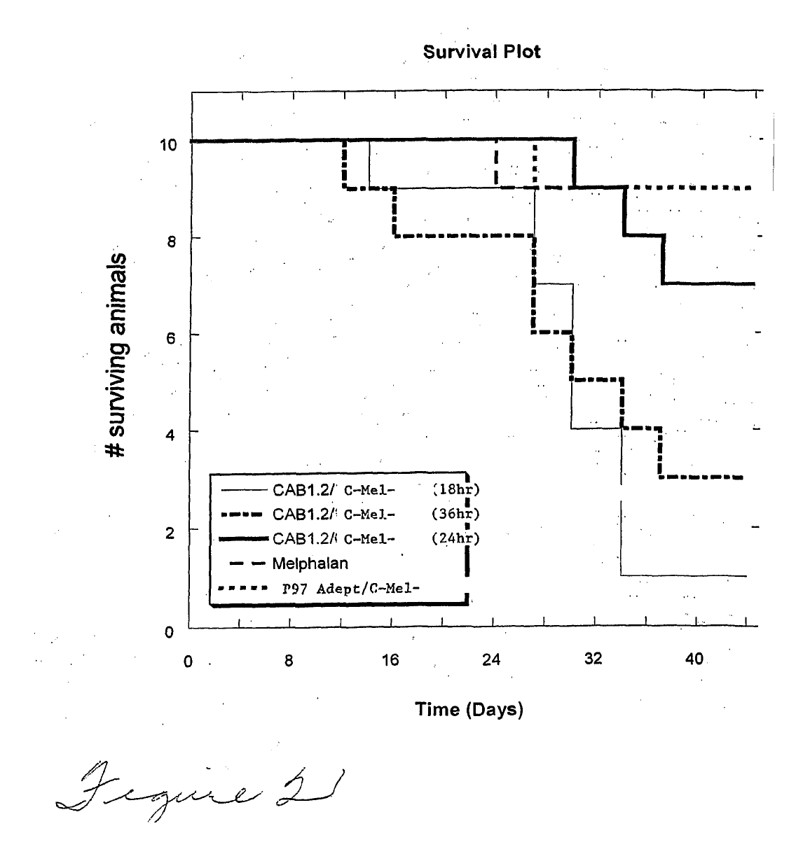

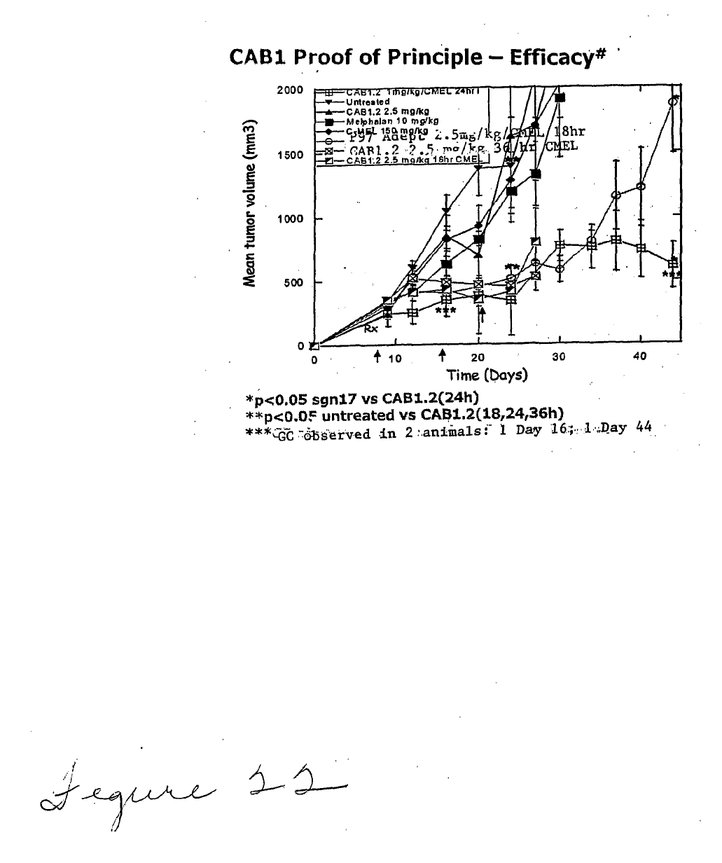

Efficacy results of the study are depicted in FIGS. 21-23. On day 8, when mice received CAB1.2 protein for the first time, mean tumor volumes for the treatment groups were approximately 177 mm³. Tumor doubling time at this phase of the study was approximately 24-26 hours, and when C-Mel was administered 18-36 hours later, the volumes of the tumors in the CAB1.2 and P97ADEPT treatment groups had nearly doubled to approximately 315 mm³. Typically treatment groups receiving CAB1.2 plus C-Mel at 18, 24, or 36 hours after CAB1.2 administration showed greater tumor growth inhibition for the duration of the study when compared to untreated, Melphalan, C-Mel, BLA or CAB1.2 control groups. At day 24 post-tumor cell challenge, tumor growth inhibition rates for CAB1.2 treatment groups administered C-Mel 18, 24 or 36 hours later were 70, 75, and 68% of the untreated control group, respectively (p<0.05, two-tailed T-Test, assuming unequal variances for each separate analysis). Through days 15-34, a similar degree of tumor growth inhibition was noted when C-Mel was administered 18 hours after P97ADEPT dosing (˜63% growth inhibition versus untreated control on day 24, p<0.05) suggesting nonspecific intratumoral retention of the BLA fusion protein. At later times, there was a separation in tumor growth inhibition rates for the CAB1.2/C-Mel treatment groups versus the P97ADEPT/C-Mel treatment group, specifically, when comparing the 24 hour CAB1.2/C-Mel treatment group versus the P97ADEPT/C-Mel treatment group at day 44, there was a three-fold difference in mean tumor volumes that was significant (611 mm³+176 vs 1871 mm³±379, respectively, p<0.05). Using the C-Mel treatment group as a comparator on day 44, the 24 hour CAB1.2/C-Mel and 18 hour P97ADEPT/C-Mel treatment groups gave growth inhibition rates of 89 and 68%, respectively (p<0.05 vs C-Mel treatment group). The remaining animals in the CAB1.2/C-Mel 18 and 36 hour treatment groups gave results that were consistent with the CAB1.2/C-Mel 24 hour treatment group as well (87 and 89% growth inhibition, respectively). There were two animals in the 24 hour CAB1.2/C-Mel treatment group that had apparent complete regressions of measurable tumor mass (noted at day 16 and day 44). At day 34 the BLA/C-Mel treatment group had significant antitumor activity (65% tumor growth inhibition vs untreated control, p<0.05). The study was terminated on day 44.

Treatment-related toxicity as demonstrated by weight loss (FIG. 21) and animal deaths (FIG. 22) was noted in various treatment groups, including those receiving CAB1.2 plus C-Mel. The average weights of individual treatment groups taken on day 9, when the first dose of C-Mel was administered, was used as the baseline weight of tumor-bearing animals for weight loss determinations within each treatment group. The treatment group receiving C-Mel 18 hours post-CAB1.2 had a 13% weight loss at day 16 and 21% weight loss at day 24, after the third round of CAB1.2/C-Mel treatment was completed. One animal was lost at day 14 due to procedural error during treatment. Toxicity-related deaths were noted as follows: 2 animals found dead on day 27, 3 animals each found dead on days 30 and 34, for a total of 8/9 toxicity-related deaths on the study for the 18 hr CAB1.2/C-Mel treatment group. The treatment group receiving C-Mel 36 hours post-CAB12 had an 8% weight loss at day 16 and 20% weight loss at day 24. One animal was sacrificed on day 12 due to excessive necrosis at the tumor site. Toxicity-related deaths were as follows: One animal found dead on day 16, 2 animals found dead on day 27, 1 animal each found dead on days 30, 34 and 37, for a total of 6/9 toxicity-related deaths on the study for the 36 hr CAB1.2/C-Mel treatment group. The treatment group receiving C-Mel 24 hours post-CAB1.2 had 12% weight loss at day 16 and 19.7% weight loss at day 24. Toxicity-related deaths were as follows: One animal each found dead on days 30, 34, and 37, for a total of 3/10 toxicity-related deaths on the study for the 24 hr CAB1.2/C-Mel treatment group. The melphalan treatment group had 11% weight loss on day 16 and 12.5% weight loss on day 24. Toxicity-related deaths were as follows: One animal each found dead on days 24, 34 and 37, for a total of 3/10 toxicity-related deaths on study for the melphalan alone treatment group. None of the remaining treatment groups had significant toxicity relative to the active treatment groups, although the BLA/C-Mel treatment group did have 16.7% weight loss noted at day 24.

CAB1.2 used in combination with C-Mel had significant tumor growth inhibitory activity in the LS174T tumor model, with >80-90% tumor growth inhibition and some tumor regressions noted in individual animals. The aggressive dose and schedule used in some of the treatment groups resulted in toxicity, which was not unexpected. Three cycles of treatment were toxic, with significant weight loss and animal deaths noted, but acceptable toxicity was noted with two cycles of therapy, particularly in the CAB1.2/C-Mel 24 hour treatment group that received 1 mg/kg of CAB1.2. Melphalan, given at a maximum tolerated dose (MTD), resulted in 30% animal deaths, which was similar to the number of deaths noted in the 24 hr CAB1.2/C-Mel treatment group, yet melphalan had significantly less efficacy than the 24 hr CAB1.2/C-Mel treatment group, with 34 vs 86% inhibition, respectively, at day 34 (p<0.05 for CAB1.2/C-Mel, p=not significant for Melphalan vs untreated control). Also, no tumor regressions occurred in the melphalan treatment group, whereas 2/10 animals in the 24 hr CAB1.2/C-Mel treatment group had complete regressions of palpable tumor mass. The MTD dose and schedule of Melphalan used in this study cannot be exceeded without causing excessive animal deaths without further improvement in tumor response. The P97ADEPT/C-Mel and BLA/C-Mel treatment groups also had significant antitumor activity, suggesting nonspecific tumor retention of these molecules at the 18 hour timepoint of C-Mel administration, but were not as effective over the duration of the study as the 24 hour CAB1.2/C-Mel treatment group.

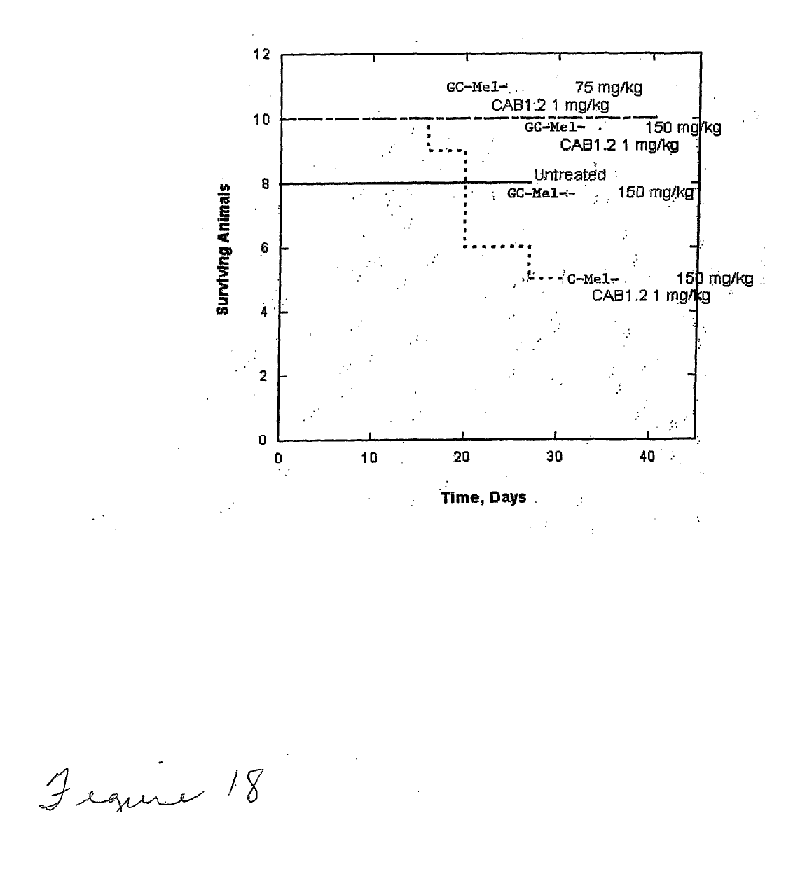

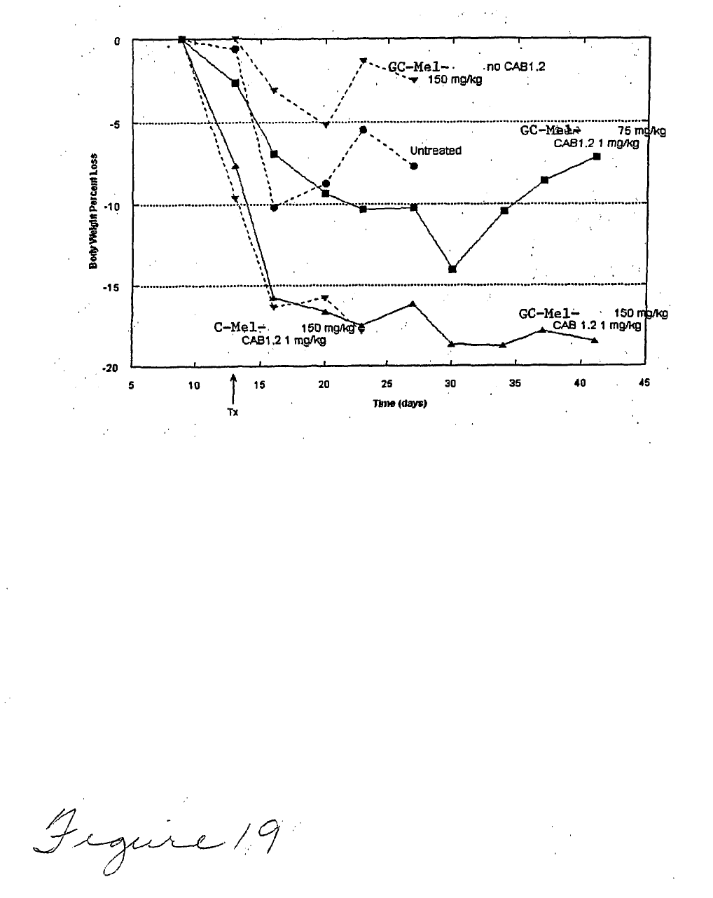

FIG. 20 shows animal weight effects after administration of CAB1.2/prodrug combinations compared with controls. The x-axis shows time in days, and the y-axis shows treatment group weight as measured in grams.

FIG. 21 plots survival of CAB1.2/prodrug combinations compared with controls. The x-axis shows time in days, and the y-axis shows the number of surviving animals.

FIG. 22 shows efficacy of the CAB1.2/prodrug combinations compared with controls. The x-axis shows time in days; and the y-axis shows tumor-volume measured in mm3. Animals received CAB1.2 and controls, as provided in the Examples, on days 8, 14 and 21. Complete responses were noted in one animal each on days 16 and 44 for Group3 (CAB1.2/C-Mel, 24 hr). Tumor volume values for these animals were scored as 0 mm3 for mean tumor volume calculations. Groups are as follows: Group 1: CAB1.2/C-Mel (2.5 mg/kg, 18 hr); Group 2: CAB1.2/C-Mel (2.5 mg/kg, 36 hr); Group 3: CAB1.2/C-Mel (1 mg/kg, 24 hr): Group 4: Untreated control; Group 5 CAB1.2 alone (2.5 mg/kg); Group 6 GCR9885 alone; Group 7 Melphalan (10 mg/kg); Group 8 P97ADEPT/C-Mel (2.5 mg/kg, 18 hr); Group 9 BLA/C-Mel (1.5 mg/kg, 18 hr).

### Example 13

### Antitumor activity of CAB1.13i and CAB1.11i in the Tumor Derived T-LS174T Human Colorectal Tumor Model in Athymic Mice

The study was performed to compare the efficacy of CAB1.13i and CAB1.11i followed by administration of glutaryl-C-Mel in tumor derived T-LS174T tumor bearing female athymic mice.

CAB1.11i was diluted to 0.05 mg/mL and 0.2 mg/mL, and CAB1.13 was diluted to 0.2 mg/mL using PBS. Doses were administered within 60 minutes of dilution.

Glutaryl-C-Mel is weighed and dissolved in 3.0 eq of 1.0 M NaHCO3. The solution is mixed well by vortex and diluted with 5% aqueous sucrose solution to 30 mg/mL final concentration. The solution is further diluted with PBS to 20 mg/mL and kept on ice packs until administered.

Study design is outlined in Table 11. Seventy female mice were implanted with tumor derived TLS174T cells by subcutaneous injection suspended in DMEM at 2×10⁷ cells/mL. Animals were anesthetized by isoflurane inhalation, and cells were implanted by subcutaneous injection of 100 uL cell suspension (approximately 2×10⁶ cells/mouse).

When tumors reached approximately ≧250 mm³, 50 animals were selected based on tumor size and growth rate and assigned into 5 groups. Ten mice each were administered nothing, or CAB1.11i (1 or 0.25 mg/kg), or CAB1.13i (1 mg/kg) followed by glutaryl-C-Mel (150 mg/kg) 24 hours after CAB administration. The CAB was formulated to 0.05 mg/mL and/or 0.2 mg/mL and glutaryl-C-Mel was formulated to 30 mg/mL. All test articles were injected within 60 minutes of dilution and formulation. Injections of approximately 100 uL/mouse were administered via the tail vein.

TABLE 11
Study Design

| | | | Dose | | Dose | |
|---|---|---|---|---|---|---|
| Group | N/Sex | CAB | (mg/kg) | Prodrug¹ | (mg/kg) | Observations |
| 1 | 10F | untreated | — | — | — | Body weight: |
| 2 | 10F | CAB1.13i | 1 | glutaryl-C- | 150 | weekly |
| | | | | Mel | | Cage side |
| 3 | 10F | CAB1.11i | 0.25 | glutaryl-C- | 150 | observations: daily |
| | | | | Mel | | Tumor |
| 4 | 10F | CAB1.11i | 1 | glutaryl-C- | 150 | Measurements: |
| | | | | Mel | | twice weekly |
| 5 | 10F | — | — | glutaryl-C- | 150 | |
| | | | | Mel | | |

¹Administered 24 hours post CAB dose

After tumor implantation, animals were observed daily at minimum and moribund or distressed animals were euthanized. Tumors were measured twice weekly, and body weights were recorded weekly.

When tumors reached ≧250 mm³, animals were assigned to groups. Mice were weighed on the day of dosing, and doses were based on the average weight of all animals. Mice were warmed with a heat lamp and heating pad and placed in a restrainer. The tail was wiped with 70% alcohol and doses were administered by bolus intravenous injection via the tail vein.

Treatment groups whose average tumor volume exceeded 2000 mm³ were euthanized, and individual animals whose tumor were excessively large and/or necrotic were euthanized. A treatment group was euthanized if fewer than 6 animals remained in the study, except to monitor individual animals that achieved a complete response for tumor regrowth. Moribund or distressed mice were sacrificed.

On Day 45, remaining mice were euthanized by CO2 inhalation and underwent necropsy. Abnormal tissues or organs were formalin fixed for histopathology. Tumors were collected from all animals into formalin for histopathology.

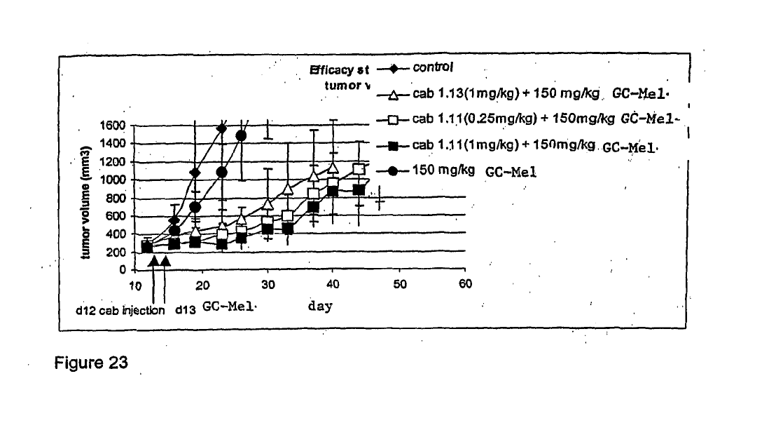

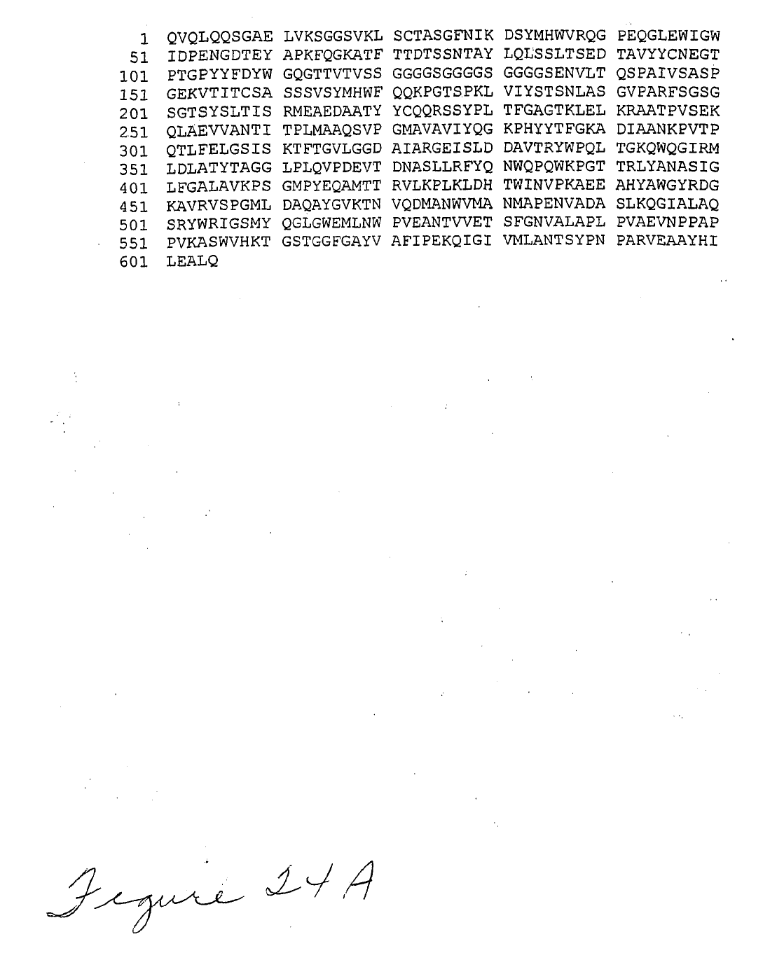

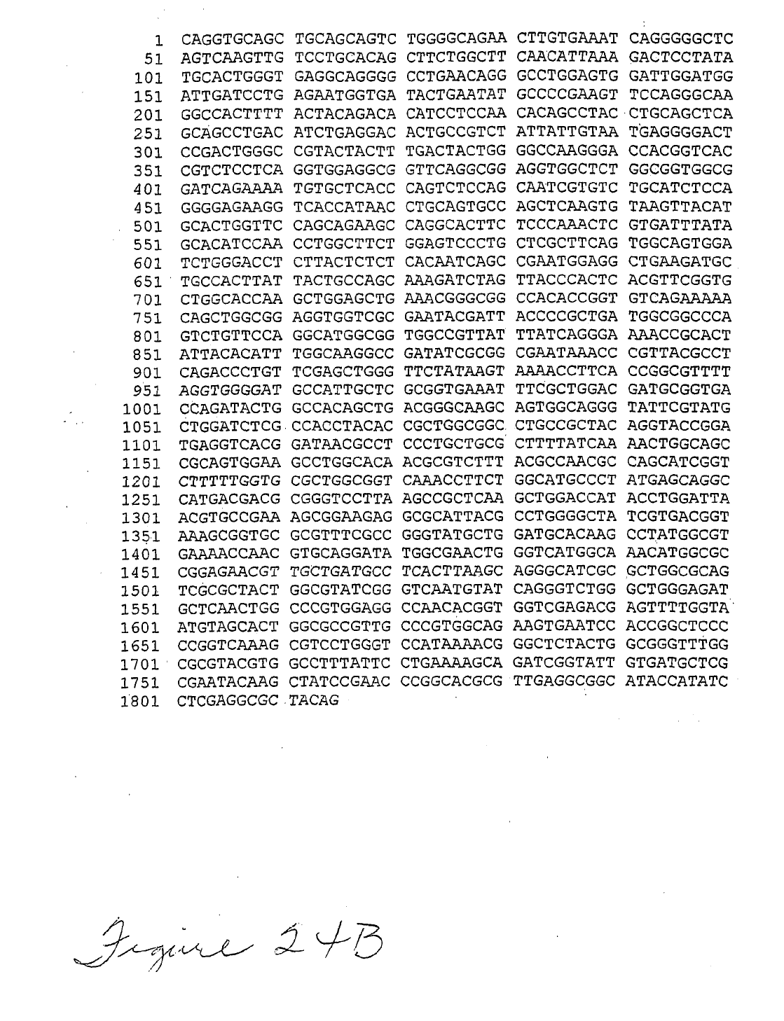

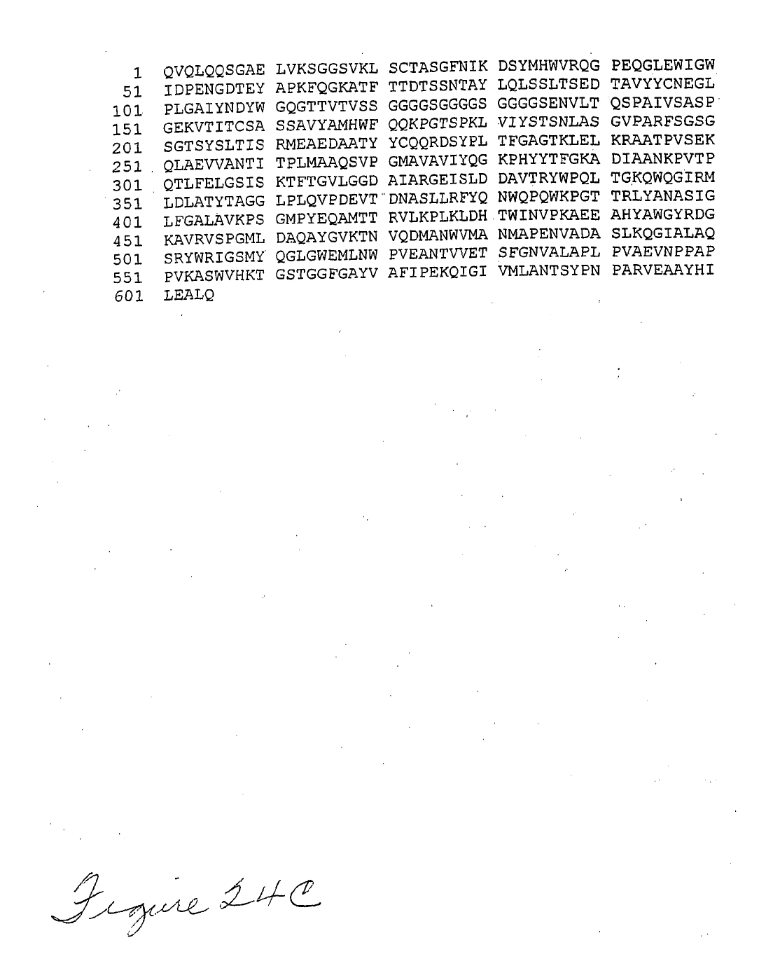

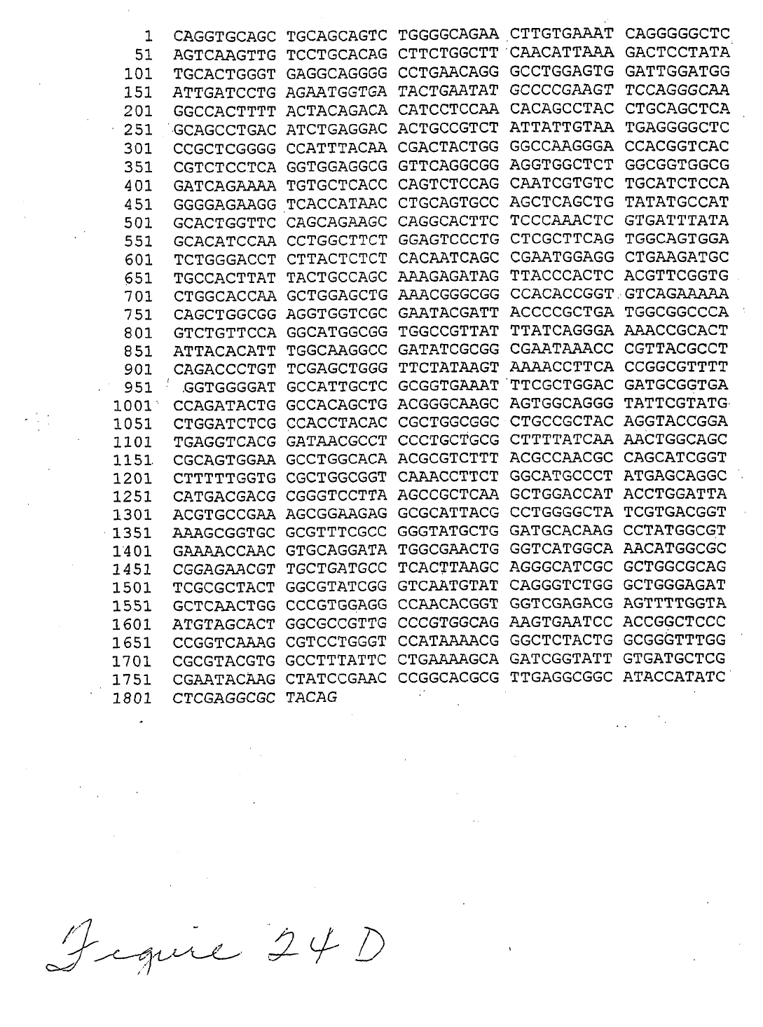

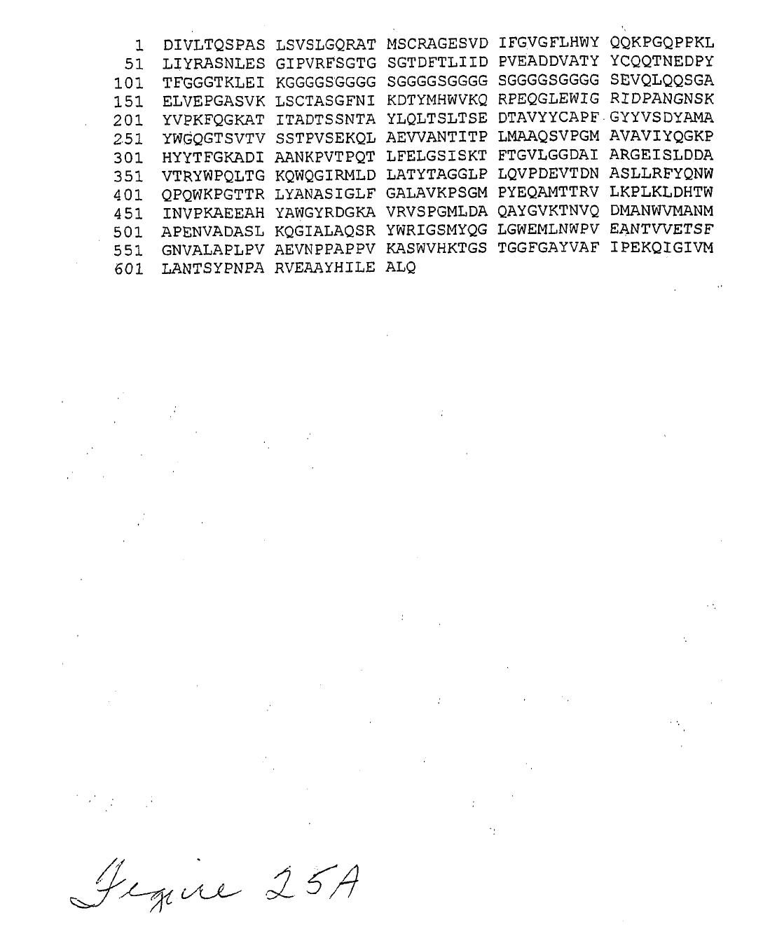

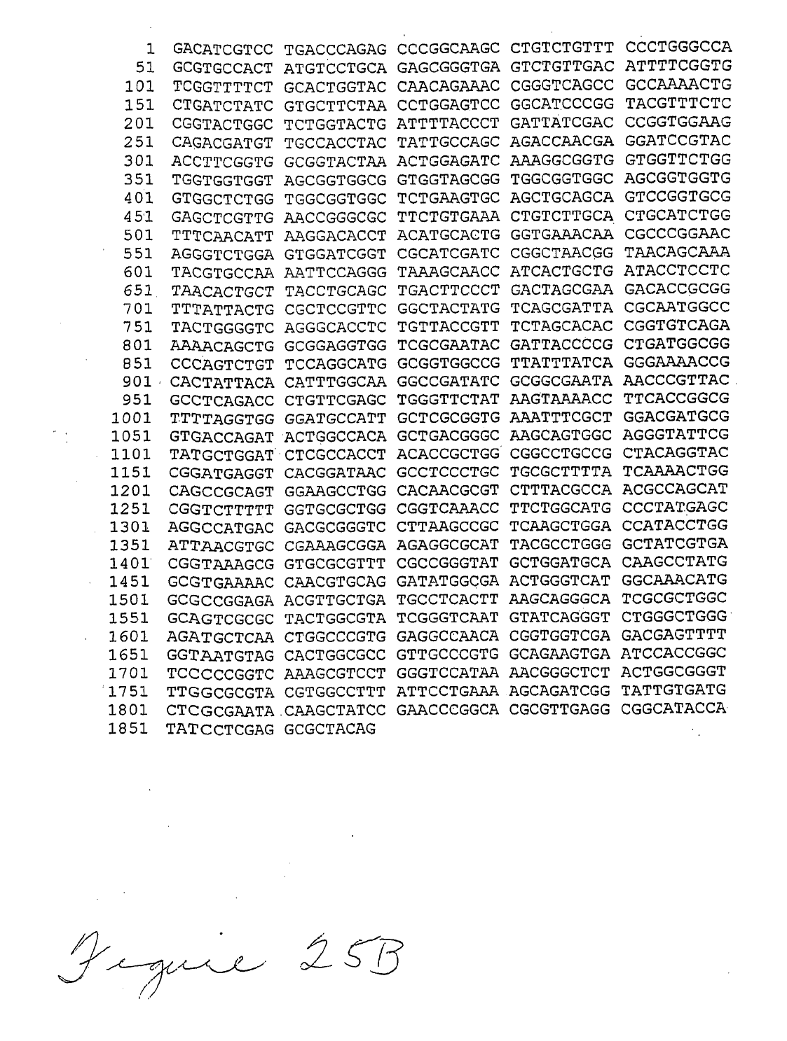

Body weight and mean tumor volumes±SD for all treatment groups were calculate, and plotted for analysis (percent body loss and days post tumor challenge vs tumor volume). Results are shown in FIG. 23.

### Example 14

### Construction of a Ropo2 Antibody

An antibody specific for BLA, Ropo2, was constructed as described. BLA was suspended in PBS Buffer (1 mg/ml), emulsified by mixing with an equal volume of Complete Freund's. Adjuvant (Total volume of 0.6 ml) and injected into three to four subcutantous dorsal sites for primary immunization. Subsequent immunizations were performed using Incomplete Freund's Adjuvant at a dose of 200 ug/rabbit. For collection, animals were bled from the articular artery. The blood was allowed to clot and serum was collected by centrifugation. Serum was stored at −20 C.

### Example 15

### Tumor Panel IHCs to Assess Distribution of Target Antigen and Binding Specificity

Frozen tissue samples used in this study were obtained from Ardais' BIGR® Library (Ardais). Genencor provided preparations of CABs as well as the rabbit polyclonal anti-BLA antibody, Ropo2. IHC analysis was used and as a positive control, a cytokeratin antibody (Dako Cytomation) was used. Please see Table 12.

TABLE 12

| Antibody | Source | Concentration | | Species |
|---|---|---|---|---|
| CAB 1.2i with 15-mer | | 1.4 | mg/ml | N/A |
| CAB 1.11i | | 1.0 | mg/ml | N/A |
| CAB 1.2i with 30-mer | | 3.0 | mg/ml | N/A |
| CAB 1.14i | | 1.8 | mg/ml | N/A |
| Ropo 2 αBLA | | 436 | μg/ml | Rabbit |
| Cytokeratin | Dako Cytomation | 0.2 | mg/ml | Mouse |

Frozen samples were removed at temperatures between −80° C. and placed in −20° C. for 2 hours. The cryostat was set at −20° C. and section samples were cut at 5 μm thickness. Sections were placed on Plus Slides and stored in a microscope slide box on dry ice while sectioning. Sections were air dried at room temperature for 30 minutes. Sections were placed in acetone at room temperature for 10 minutes. Sections were rinsed in Wash Buffer (Dako Cytomation, Code# S3006, Lot# 044312) 2-3×5 min at room temperature.

IHC was performed on a Dako autostainer. Antibodies were diluted in Antibody Diluent (Dako Cytomation, Code# S0809, Lot# 123113) to the following concentrations: CAB antibodies to 0.2 μg/ml and Ropo 2 antibody to 0.1 μg/ml. Samples were incubated with approximately ˜200 μl Peroxidase Block for 5 minutes at room temperature. Antibodies were rinsed with wash buffer for 2×5 minutes. Samples were incubated with approximately ˜200 μl Protein Block (Dako Cytomation, Code #X0909, Lot# 103183) for 10 minutes. ˜200 μl CAB antibody was added for 30 minutes at room temperature. Samples were washed with Wash Buffer 2×5 minutes. Approximately ˜200 μl Ropo 2 antibody was added and incubation occurred for 30 minutes at room temperature. Samples were rinsed with Wash Buffer for 2×5 minutes. ˜200 μl Secondary Antibody from Detection System was added and incubated for 30 minutes. The samples were rinsed with wash buffer for 2×5 minutes. Samples were incubated in ˜200 μl Chromagen (DAB+ provided in Detection System (Envision+ System, HRP (DAB) Rabbit)—Dako Cytomation, Code# K4011, Lot# 11367)) for 5 minutes. The samples were washed with distilled water for 5 minutes. The samples were counterstained with Hematoxylin (Richard Allen, Code# 7211, Lot# 35053), which provides a blue nuclear stain, for 30 seconds. The samples were rinsed for 5 minutes. Samples were dipped twice in a Bluing Reagent (Richard Allen, Code# 7301, Lot# 19540). Samples were rinsed with distilled water for 5 minutes. Samples were dehydrated in 95% Ethanol 2×2 minutes, 100% Ethanol 2×2 minutes and cleared in Xylene. Samples were mounted with Medium (Richard Allen, Code# 4111, Lot# 18071), and a coverslips were added.

In this IHC study, the four CAB antibodies CAB 1.2i, 15-mer linker, CAB 1.2i, 30-mer linker, CAB 1.11i and Cab 1.14i were analyzed against a tissue panel consisting of 5 lung, 3 colon, and 5 pancreatic tumor samples.

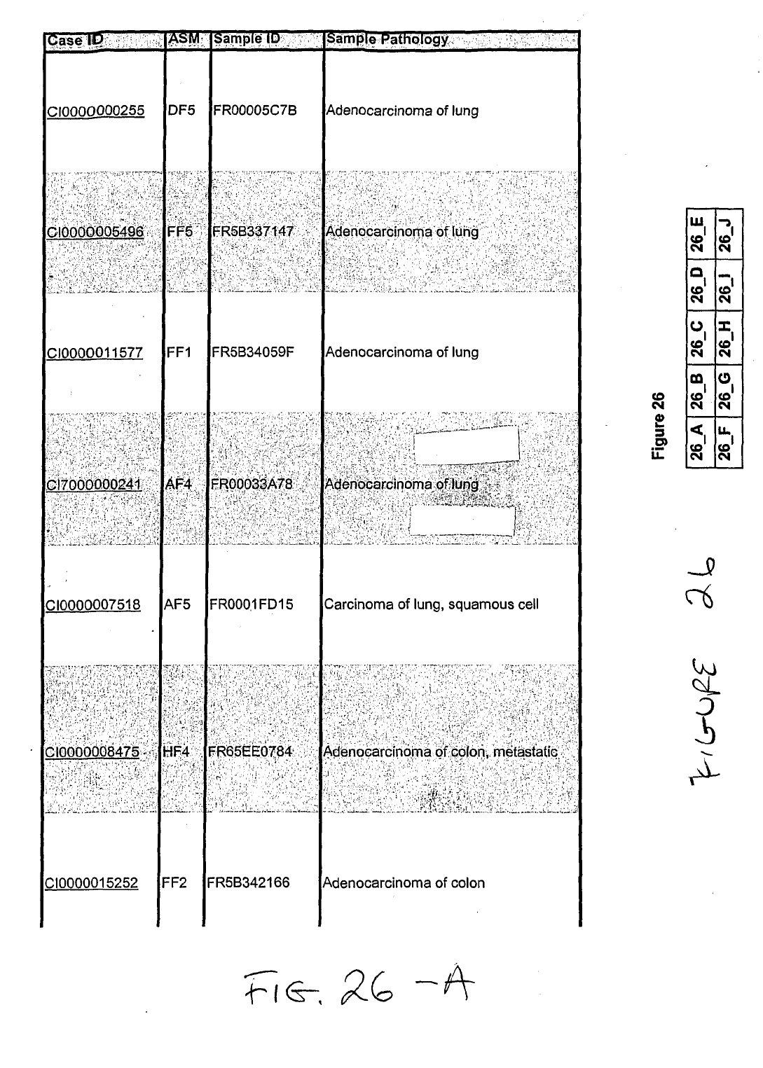

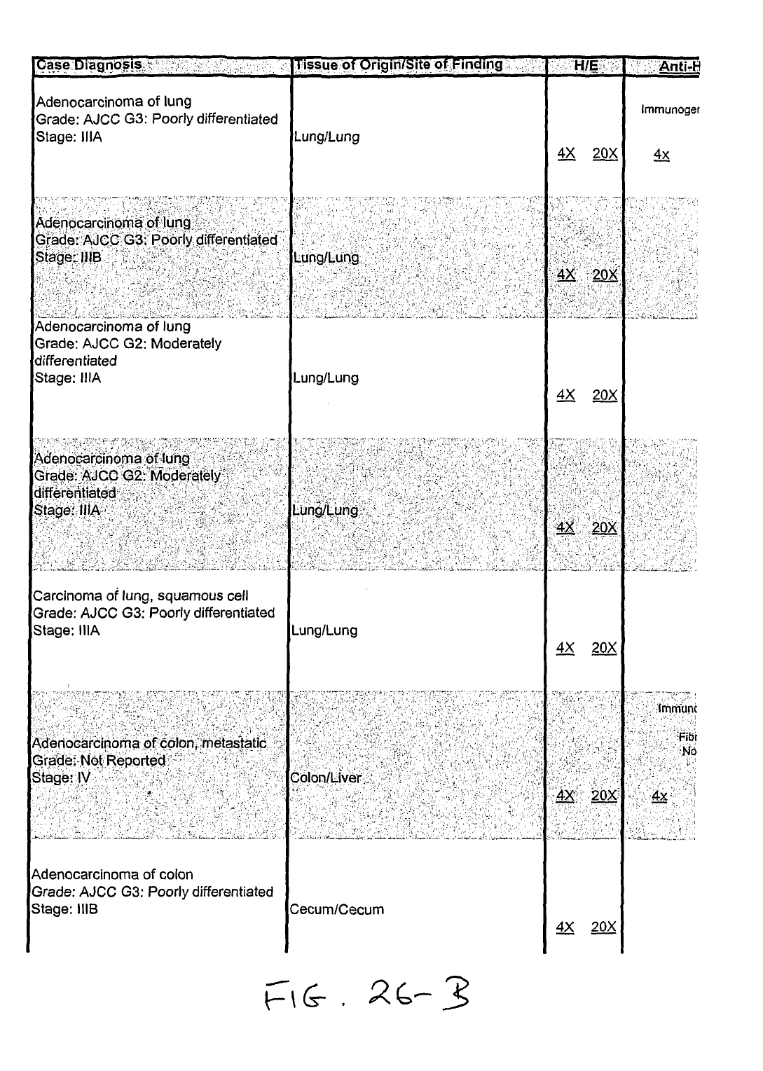

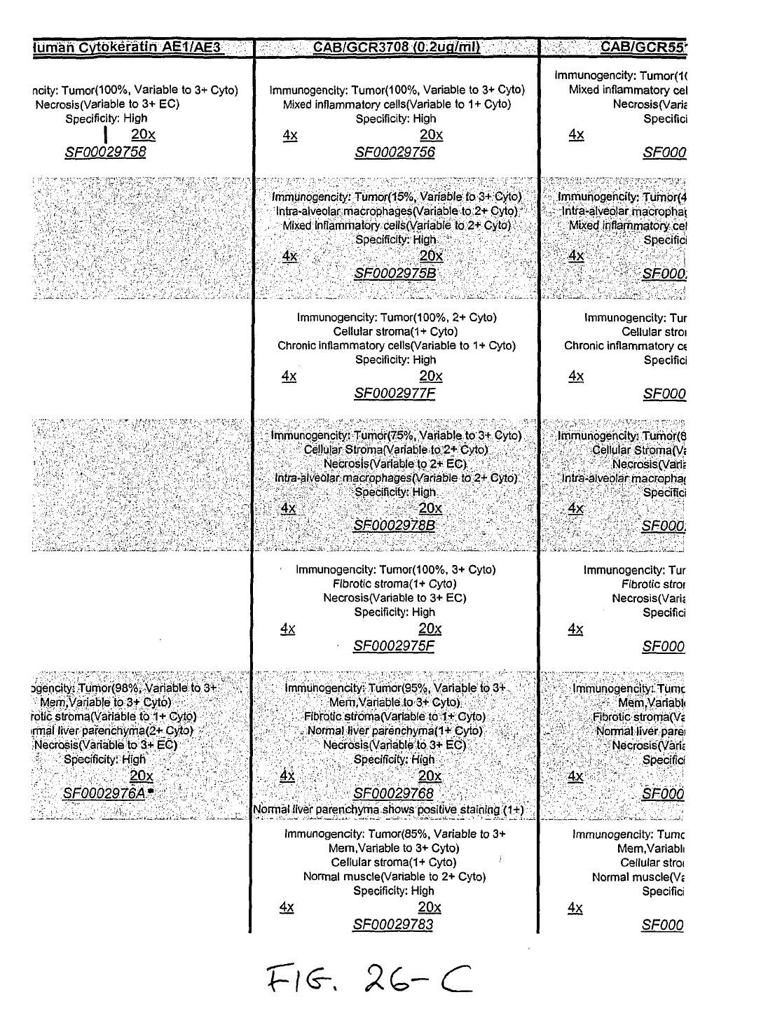

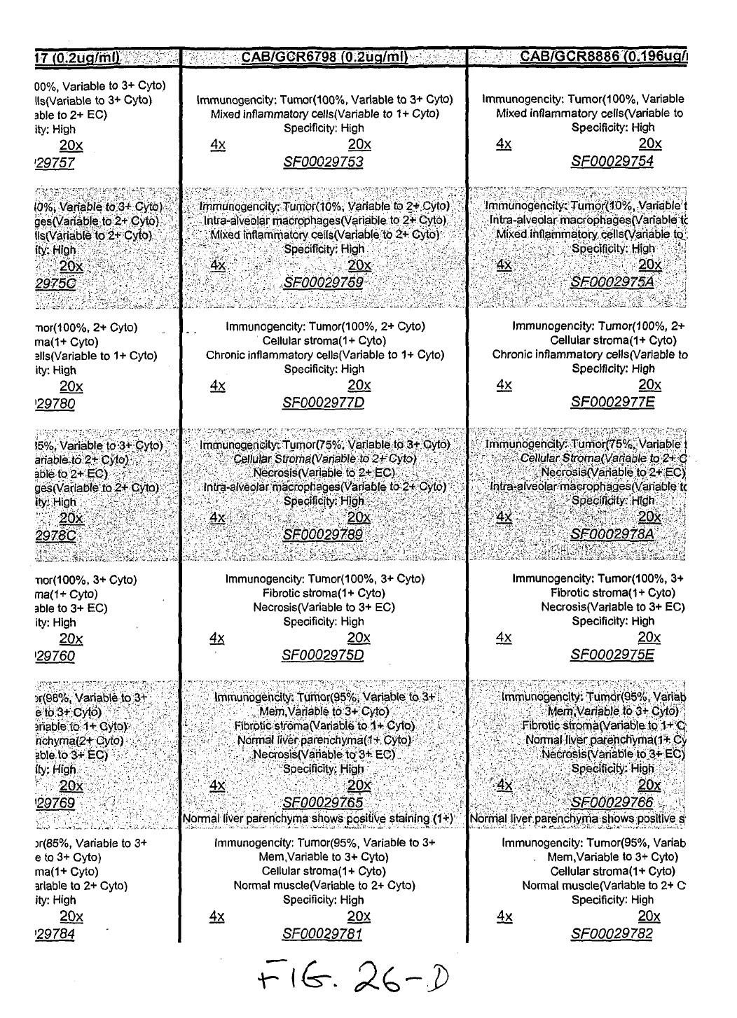

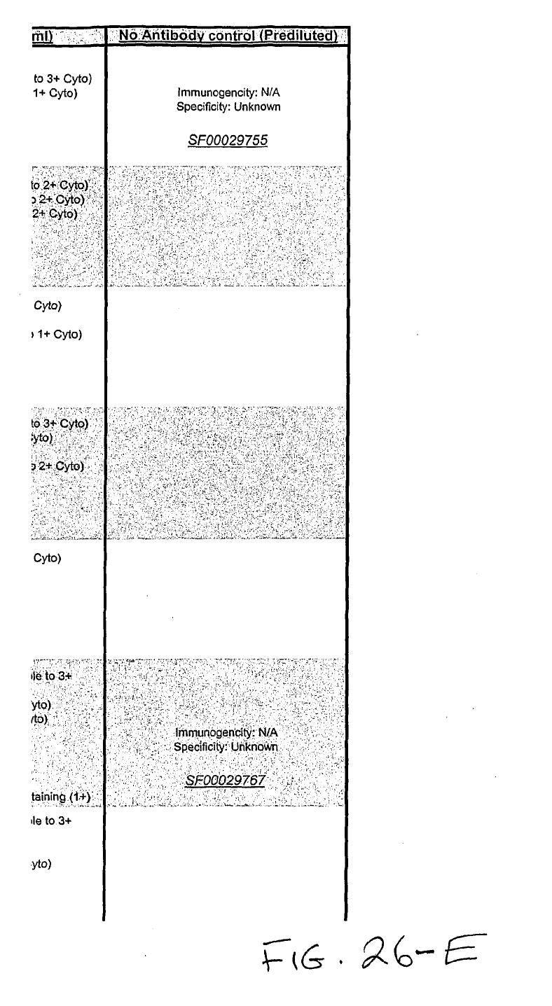

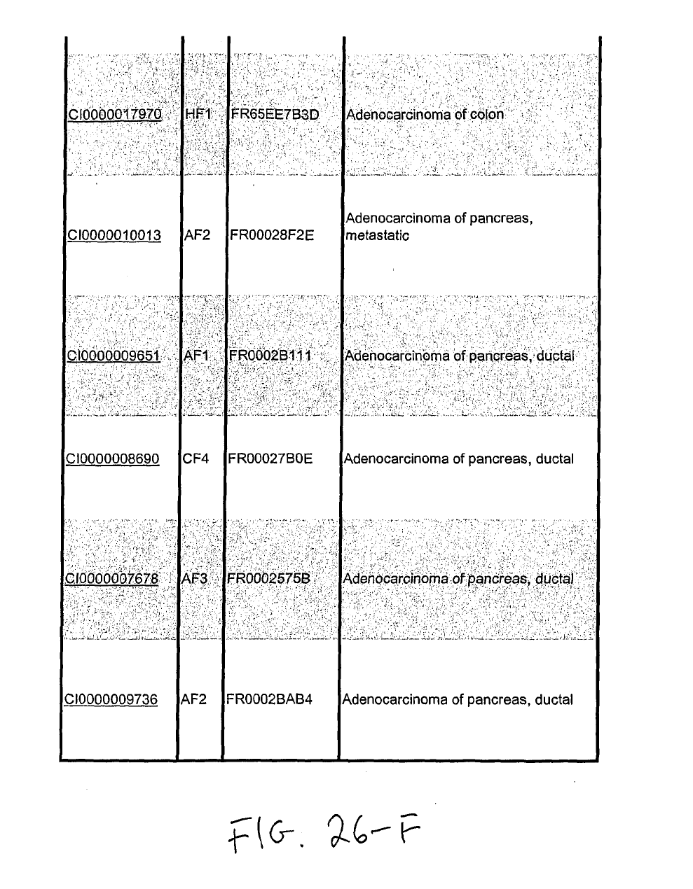

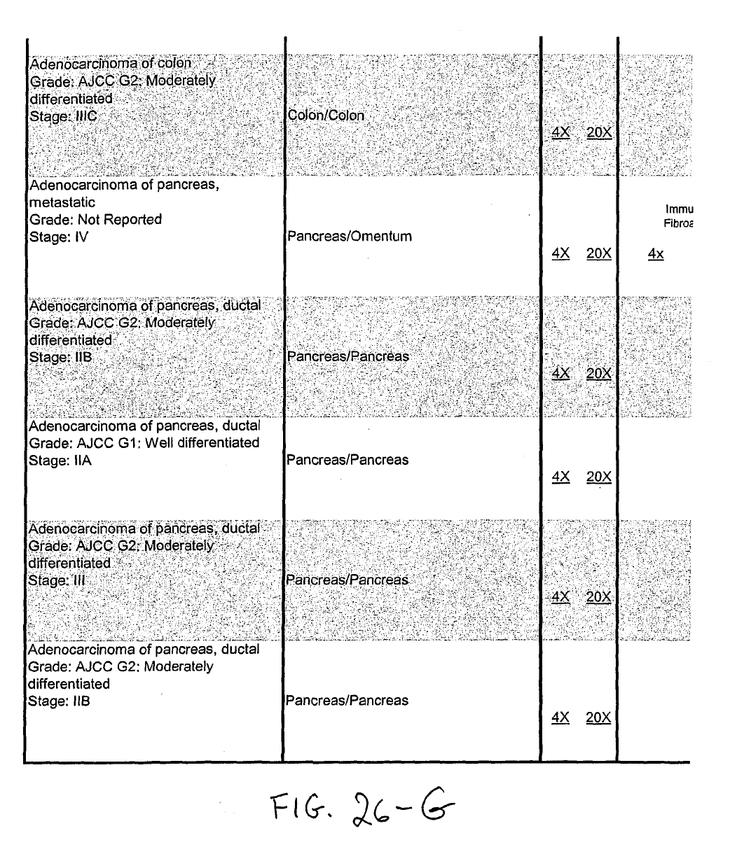

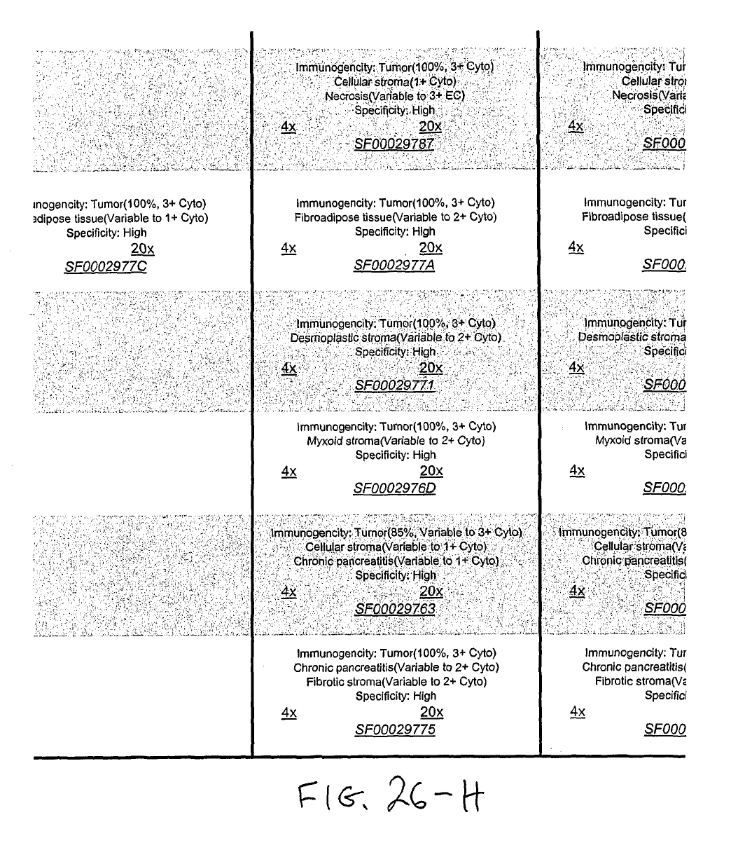

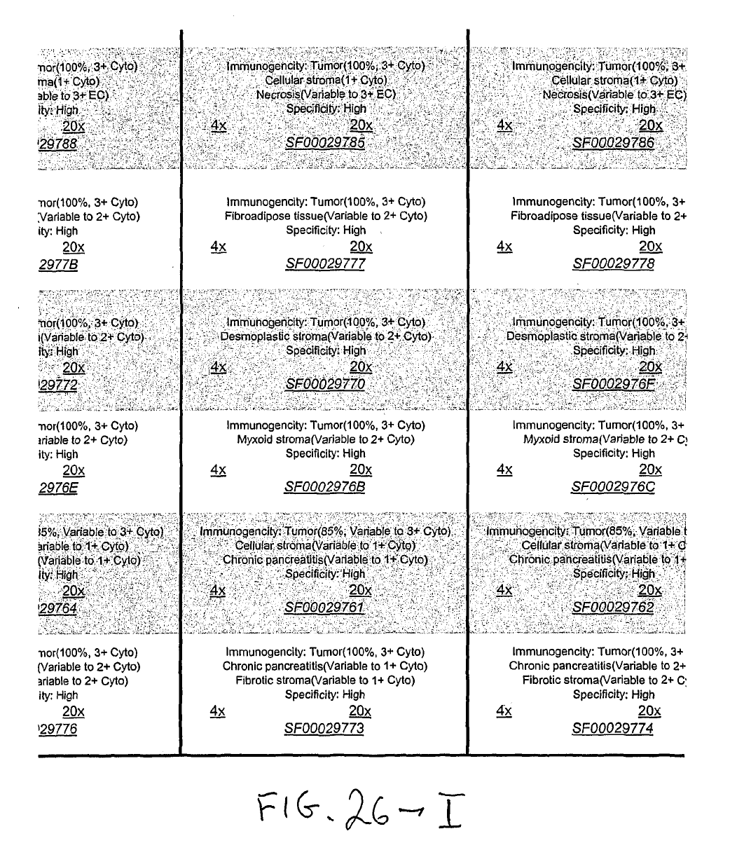

FIG. 26 shows the full results of the study. The first column details the case so diagnosis; the second column details the tissue of origin and site of finding; the fourth column shows staining with the anti-human cytokeratin AE1/AE3, columns five through eight show staining against the four antibodies, CAB 1.2i with a 15-mer linker, CAB 1.2i with a 30-mer linker, CAB 1.11i and CAB1.14i.

The four antibodies showed robust immunostaining (intensity of 2-3+) in all of the tumor samples tested and were very similar if not identical in their staining patterns. All samples with the exception of one, CI000005496-FF5, demonstrated staining in greater than 75% of tumor cells present. Minimal, pale (1-2+) staining, which is sometimes seen with frozen tissue sections, was also observed in stromal cells, including fibroblasts and occasional mixed inflammatory cells. Necrotic cells and intra-alveolar macrophages (seen in samples of lung tissue) consistently showed positive staining.

Adjacent normal tissue present in the samples was largely negative, with no positive staining seen lung or pancreatic tissue. Normal liver tissue seen in sample CI0000008475, a case of colon cancer metastatic to the liver, showed pale staining that was limited to the sinusoidal regions with 3 of the antibodies (CAB 1.2i 15-mer linker, CAB 1.11i, and CAB 1.2i, 30-mer linker). The fourth antibody (CAB 1.14i) showed stronger, more diffuse staining of 90% of normal liver parenchyma.

In comparing the staining characteristics of the four antibodies tested, there was only minimal variability observed. Of the four antibodies tested, CAB 1.14i appeared to show slightly more background staining.

The cytokeratin antibody, which was used on selected samples to ensure that the tissue antigens were properly preserved, showed strong positive staining of epithelial cells. There was no staining seen in the ‘no-primary antibody’ controls.

### Example 16

### Antitumor Activity of CAB 1.2i, 15-mer, CAB 1.2i 30-mer CAB 1.14i and Cab 1.11i Followed by Administration of GC-Mel in the Tumor-Derived TLS174T Tumor Bearing Female Athymic Mice

### Formulation:

Dosing solutions were prepared on the day of dosing, within 60 minutes of administration. An aliquot of each formulated dosing solution was retained and stored at −70° C. prior to analysis. CABs were analyzed for protein concentration and BLA activity. GC-Mel and Mel were analyzed for compound concentration.

### Preparation of GC-Mel

Bulk drug was weighed and dissolved in 3.0 eq of 1.0 M NaHCO₃. Solutions were mixed well by vortex and diluted with 5% aqueous sucrose solution to 30 mg/mL final concentration, as above. Animals received 100 μL formulated dosing solution.

### Preparation of Mel

Bulk drug was weighed and dissolved in 20% DMSO in acidified PBS (pH 4.0) to 2 mg/mL final concentration. Animals received 100 uL each formulated dosing solution.

### Species/Strain/Age/Number/Source

One hundred and fifty female Ncr athymic mice, 18-22 g, approximately 6-8 weeks, from Taconic Labs were implanted with TLS174T human colorectal tumors. One hundred animals were selected for dose administration based on tumor size and growth rate.

### Study Design

Study design is outlined in Table 13. Mice were implanted with TLS174T cells (Study Day 0) and when tumors reached approximately ≧250 mm³, 100 animals were selected based on tumor size and growth rate and sorted into 10 groups resulting in similar mean tumor size between groups. Ten mice each were administered CAB 1.2i, 15-mer, CAB 1.14i or CAB 1.11i (1 or 0.25 mg/kg) or CAB 1.2i, 30-mer (0.25 mg/ml) followed by GC-Mel (150 mg/kg) 24 hours after CAB administration. Ten mice each were administered vehicle, Mel (10 mg/kg) or GC-Mel (150 mg/kg).

TABLE 13

| | | | | GC-Mel | |
|---|---|---|---|---|---|
| | | Test | Dose | Dose² | |
| Group | N/Sex | Article | (mg/kg) | (mg/kg) | Observations |
| 1 | 10/F | Vehicle¹ | — | — | Body weight: weekly |
| 2 | 10/F | Mel | 10 | — | Cage side |
| 3 | 10/F | CAB 1.2i | 0.25 | 150 | observations: daily |
| 4 | 10/F | — | — | 150 | Tumor Measurements: |
| 5 | 10/F | CAB 1.2i, | 0.25 | 150 | twice weekly |
| | | 15-mer | | | |
| 6 | 10/F | CAB 1.2i, | 1 | 150 | |
| | | 15-mer | | | |
| 7 | 10/F | CAB 1.11i | 0.25 | 150 | |
| 8 | 10/F | CAB 1.11i | 1 | 150 | |
| 9 | 10/F | CAB 1.14i | 0.25 | 150 | |
| 10 | 10/F | CAB 1.14i | 1 | 150 | |

¹Five animals will be administered 1:10 dilutions in PBS of 20 mM sodium citrate, 150 mM NaCl, pH 6.0 and five animals will be administered 20% DMSO in acidified PBS (pH 4.0)
²GC-Mel administered 24 hours post-CAB administration

### Tumor Implantation

One hundred and fifty female mice were implanted with TLS174T cells by subcutaneous injection suspended in DMEM at 2×10⁷ cells/mL. Animals were anesthetized by isofluorane inhalation, and cells were implanted by subcutaneous injection of 100 μL cell suspension (approximately 2×10⁶ cells/mouse). The day of implantation was designated as Study Day 0.

### Dosing, Observations and Sample Collection:

After tumor implantation, animals were observed daily at minimum and moribund or distressed animals were euthanized. Tumors were measured twice weekly, and body weights were recorded weekly.

When tumors reached ≧250 mm³, animals were assigned to groups. Mice were weighed on the day of dosing, and doses were based on the average weight of all animals. Mice were warmed with a heating lamp and heating pad and placed in a restrainer. The tail was wiped with 70% alcohol and doses were administered by bolus intravenous injection via the tail vein.

Treatment groups whose average tumor volume exceeded 1500 mm³ were euthanized, and individual animals whose tumor was excessively large and/or necrotic were euthanized. A treatment group was euthanized if fewer than 6 animals remain in the study, except to monitor individual animals that achieved a complete response for tumor regrowth.

On Day 45, remaining mice were euthanized by CO₂ inhalation and underwent necropsy. Abnormal tissues or organs were formalin fixed for histopathology. Tumors were collected from all animals into formalin for histopathology.

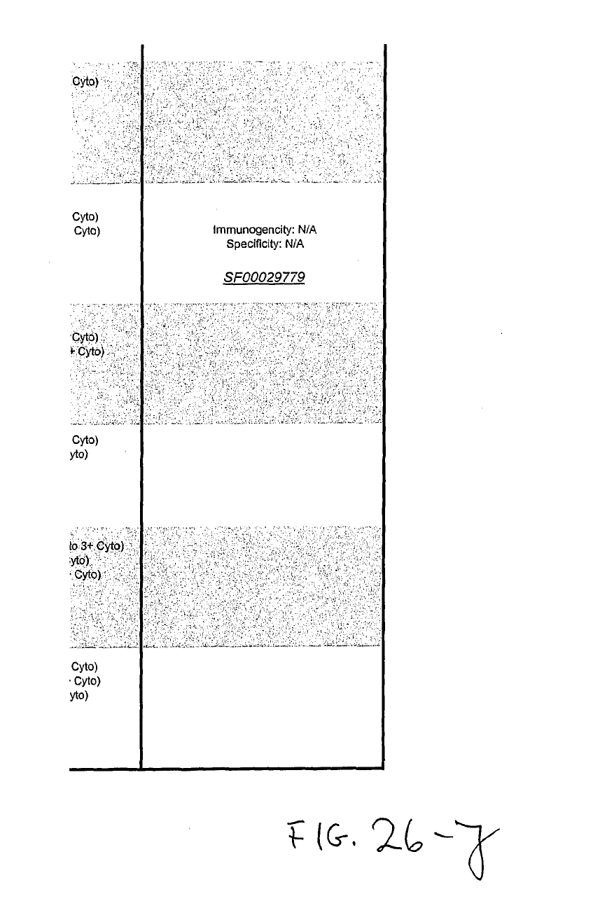

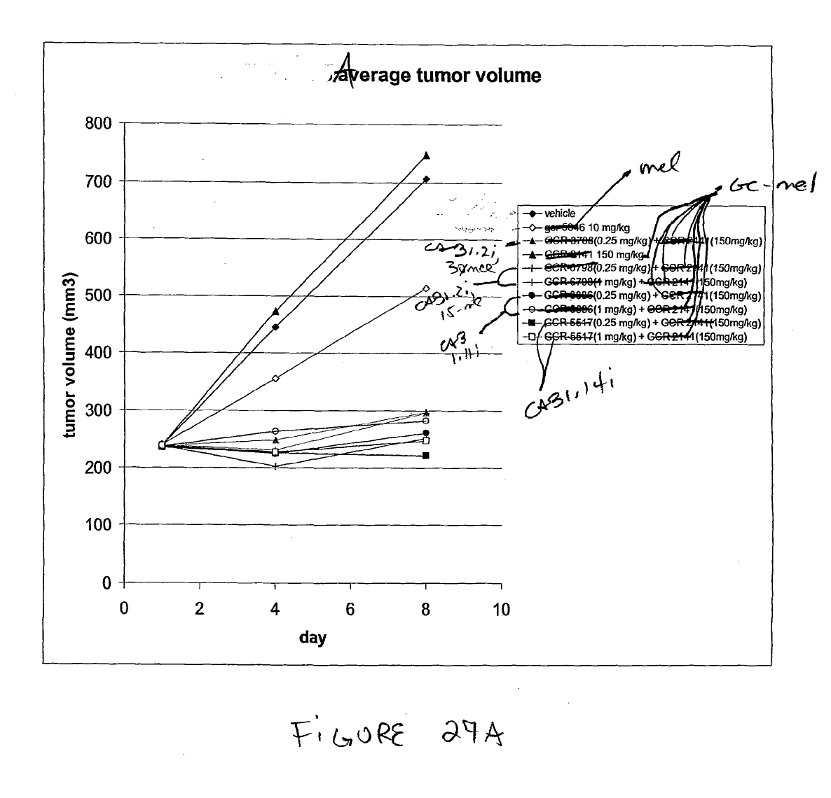

Results can be seen in FIG. 27. The CABs, followed by administration of prodrug, showed a decrease in tumor volume. However, the same grow, showed some weight loss.

### Example 17

### Pharmacokinetics and Tissue Distribution of GC-Mel at 24 hr Intervals Following Administration of CAB 1.14i or CAB 1.2i, 15-mer, in TLS174T Xenograft Bearing NCR Nude Mice

### Formulation:

Dosing solutions were prepared on the day of dosing and used within two hours of formulation. Dose concentrations were based on the average weight of all mice and formulated to deliver 100 μL/mouse.

### Species/Strain/Age/Number/Source:

Ncr athymic mice, approximately 8-10 weeks of age, from Taconic Labs were implanted with TLS174T human colorectal tumors. TLS174T is a cell line established from LS174T passaged through mice. LS174T cell line was originally purchased from ATCC. TLS174T cells are routinely tested negative for mycoplasma contamination. One hundred and twenty six animals were selected for dose administration based on tumor size and growth rate.

### Study Design:

The study design is outlined in Table 14. Animals were administered nothing, CAB 1.14i (1 mg/kg) or 1.2i, 15-mer (1 mg/kg) by intravenous injection. At 24, 48, 72 or 96 hours after CAB administration, animals received a single bolus dose of GC-Mel (100 mg/kg). Plasma, tumor, liver and kidney were collected over 60 minutes post GC-Mel administration for analysis of GC-Mel concentrations.

TABLE 14
Study Design

| | | | | GC-Mel | | | |
|---|---|---|---|---|---|---|---|
| | | Test | Dose | Dose | | Timepoints² | Sample |
| Group | N/Sex | Article | (mg/kg) | (mg/kg) | TOA¹ | (min) | Collection |
| 1 | 3/F | None | — | — | — | 0 | Plasma, |
| 2 | 15/F | CAB 1.14i | 1 | 100 | 24 | 2, 5, 15, 30, | Tumor, |
| 3 | 15/F | CAB 1.14i | 1 | 100 | 48 | 60 | Liver, and |
| 4 | 15/F | CAB 1.14i | 1 | 100 | 72 | | Kidney |
| 5 | 15/F | CAB 1.14i | 1 | 100 | 96 | | |
| 6 | 3/F | None | — | — | — | 0 | |
| 7 | 15/F | CAB1.2i, | 1 | 100 | 24 | 2, 5, 15, 30, | |
| | | 15-mer | | | | 60 | |
| 8 | 15/F | CAB1.2i, | 1 | 100 | 48 | | |
| | | 15-mer | | | | | |
| 9 | 15/F | CAB1.2i, | 1 | 100 | 72 | | |
| | | 15-mer | | | | | |
| 10 | 15/F | CAB1.2i, | 1 | 100 | 96 | | |
| | | 15-mer | | | | | |

¹Time of administration, post CAB1.2i, 15-mer, or CAB 1.14i administration
²Collected post GC-Mel administration

### Tumor Implantation:

Mice were implanted with TLS174T cells by subcutaneous injection suspended in DMEM at 2×10⁷ cells/mL. Animals were anesthetized by isoflurane inhalation, and cells were implanted by subcutaneous injection of 100 μL cell suspension (approximately 2×10⁶ cells/mouse).

### Dosing, Observations and Sample Collection:

After tumor implantation, animals were observed daily at minimum and moribund or distressed animals were euthanized. Tumors were measured twice weekly, and body weights was recorded weekly.

When tumors reached approximately 200-400 mm³, animals were assigned to groups. Mice were weighed on the day of dosing, and doses were based on the average weight of all animals. Mice were warmed with a heating lamp and heating pad, placed in a restrainer and doses were administered by bolus intravenous injection via the tail vein.

All mice were anesthetized by isofluorane inhalation at the time of sample collection. Blood was collected by cardiac puncture into tubes containing EDTA and placed on ice. Tubes were centrifuged at 13,000 RPM for two minutes. The plasma fraction was removed into a pre-labeled microfuge tubes and placed on dry ice. All plasma samples were stored at −70° C. prior to analysis.

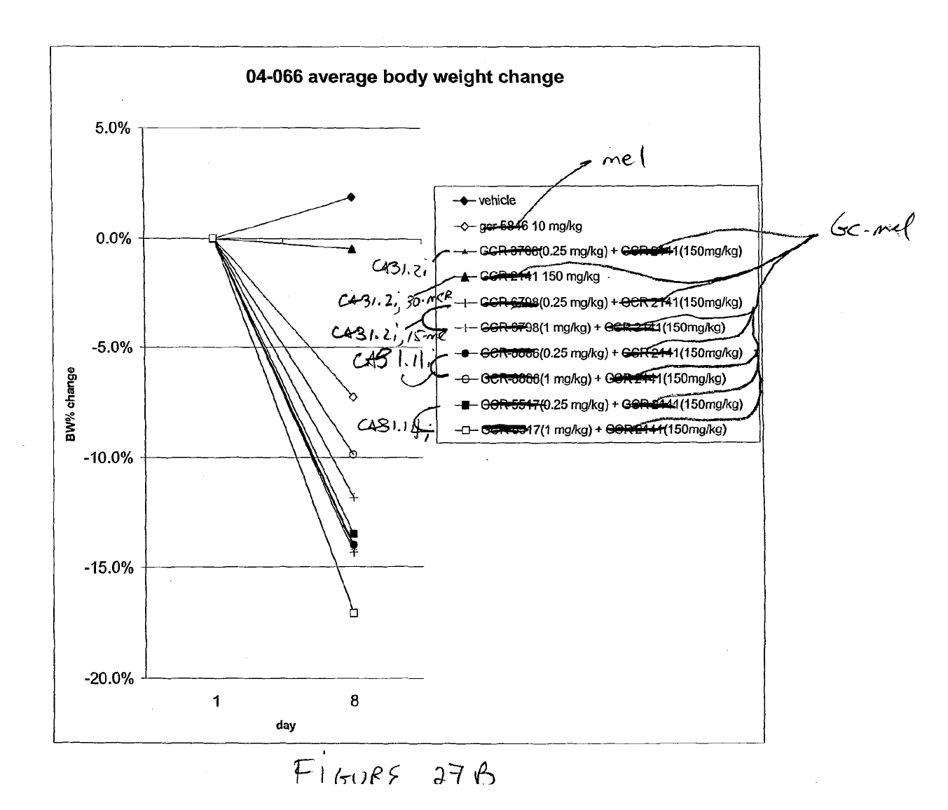

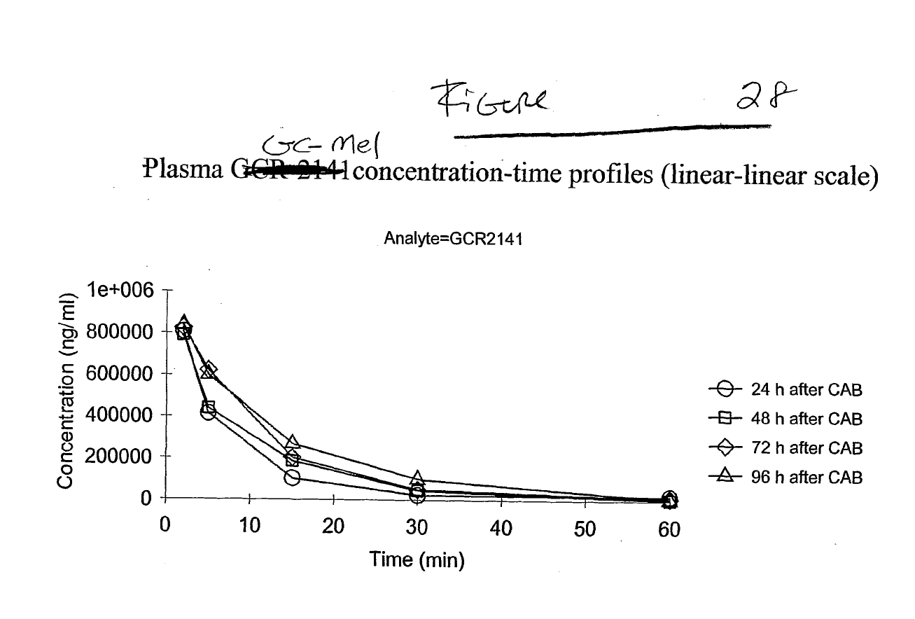

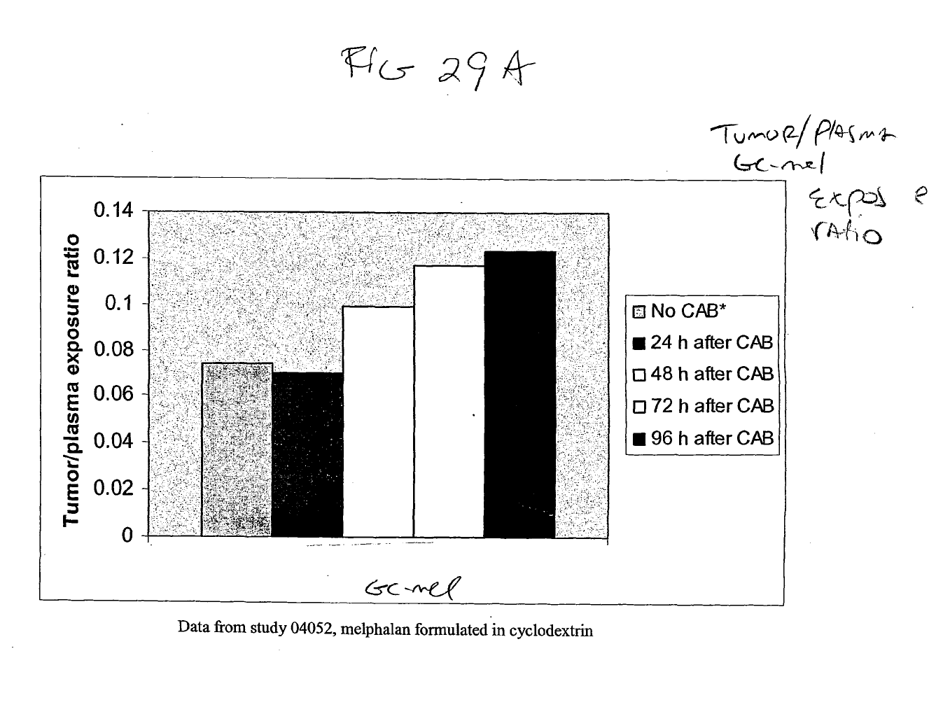

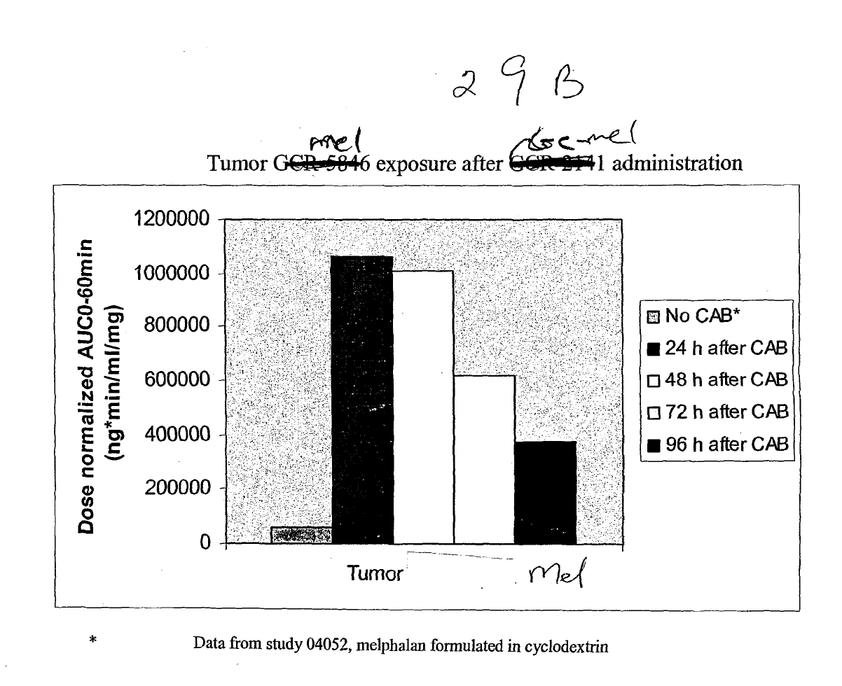

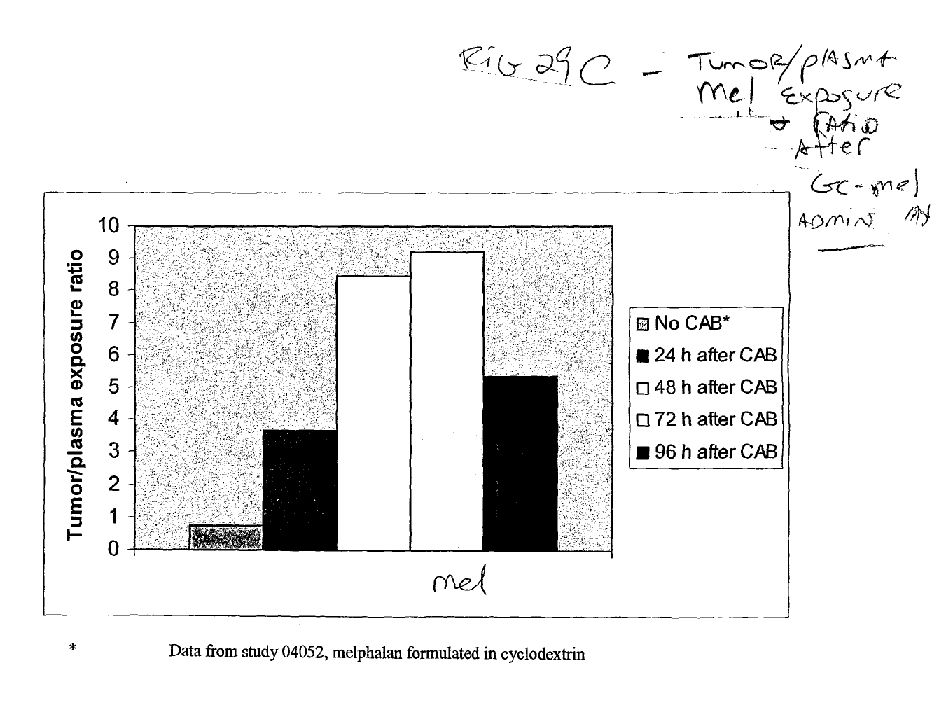

Plasma samples were assayed for plasma GC-Mel concentration by LC/MS/MS. The results are shown in FIGS. 28-30. Having described the preferred embodiments of the present invention, it will appear to those ordinarily skilled in the art that various modifications may be made to the disclosed embodiments, and that such modifications are intended to be within the scope of the present invention.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference. Specifically, Attorney Docket Number(s) 839 et seq (e.g., 839-2P) are herein incorporated by reference, herein, in their entirety, including any drawings.

The invention illustratively described herein suitably may be practiced in the absence of any element or elements, limitation or limitations that is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A CAB molecule comprising an amino acid sequence having the sequence set forth in SEQ ID NO:1.

2. A CAB molecule comprising an amino acid sequence modified from the amino acid sequence set forth in SEQ ID NO:1, wherein the modification is at least one position selected from the group consisting of positions 100, 102, 104, 105, 107, 163, 165, 166, 184 and 226, wherein position numbering is with respect to SEQ ID NO:1 as shown in FIG. 1.

3. The CAB molecule according to claim 2, wherein the modifications are at positions 100, 184 and 226.

4. The CAB molecule according to claim 2, wherein the modifications are at positions 100, 102, 104, 105, 107, 163, 165, 166, 184 and 226.

5. The CAB molecule according to claim 2, wherein the modification is at least one selected from the group consisting of T100L, T102L, P104A, Y105, F107N, S163A, S165Y, Y166A, S184D and S226D.

6. The CAB molecule according to claim 5, wherein the CAB molecule comprises the following modifications: T100L, S184D and S226D.

7. A CAB molecule, the CAB molecule comprising an amino acid sequence having the sequence set forth in SEQ ID NO:2 as shown in FIG. 2.

8. The CAB molecule according to claim 7 further comprising at least one modification selected from the group consisting of 3, 13, 16, 37, 100, 102, 104, 105, 107, 146, 163, 165, 166, 181, 184, 226, 265 and 568, where position numbering is with respect to SEQ ID NO:2, as shown in FIG. 2.

9. The CAB molecule according to claim 8, further comprising at least one modification selected from the group consisting of K3Q, R13K, T16G, L37V, T100L, T102L, P104A, Y105, F107N, M146V, S163A, S165Y, Y166A, W181V, S184D, S226D, K265A and S568A.

10. The CAB molecule according to claim 9, further comprising SEQ ID NO:7, SEQ ID NO:8, SEQ ID NO:9 or SEQ ID NO:10.

11. The CAB molecule according to claim 9, further comprising the sequence set forth in SEQ ID NO:7.

12. The CAB molecule according to claim 9, further comprising the sequence set forth in SEQ ID NO:8.

13. The CAB molecule according to claim 9, further comprising the sequence set forth in SEQ ID NO:9.

14. The CAB molecule according to claim 9, further comprising the sequence set forth in SEQ ID NO:10.

15. A nucleic acid encoding a CAB molecule comprising an amino acid sequence having the sequence set forth in SEQ ID NO:1.

16. A nucleic acid encoding a CAB molecule having an amino acid sequence modified from the amino acid sequence set forth in SEQ ID NO:1, wherein the modification is at least one position selected from the group consisting of positions 100, 102, 104, 105, 107, 163, 165, 166, 184 and 226, wherein position numbering is with respect to SEQ ID NO:1 as shown in FIG. 1.

17. The nucleic acid according to claim 16, wherein the modifications are at positions 100, 184 and 226.

18. The nucleic according to claim 16, wherein the modifications are at positions 100, 102, 104, 105, 107, 163, 165, 166, 184 and 226.

19. The nucleic acid according to claim 18, wherein the modification is at least one selected from the group consisting of T100L, T102L, P104A, Y105, F107N, S163A, S165Y, Y166A, S184D and S226D.

20. The nucleic acid according to claim 19, wherein the CAB molecule comprises the following modifications: T100L, S184D and S226D.

21. A nucleic acid encoding a CAB molecule, the CAB molecule comprising an amino acid sequence having the sequence set forth in SEQ ID NO:2.

22. The nucleic acid according to claim 21, further comprising at least one modification selected from the group consisting of 3, 13, 16, 37, 100, 102, 104, 105, 107, 146, 163, 165, 166, 181, 184, 226, 265 and 568, where position numbering is with respect to SEQ ID NO:2, as shown in FIG. 2.

23. The nucleic acid according to claim 22, further comprising at least one modification selected from the group consisting of K3Q, R13K, T16G, L37V, T100L, T102L, P104A, Y105, F107N, M146V, S163A, S165Y, Y166A, W181V, S184D, S226D, K265A and S568A.

24. The nucleic acid according to claim 23, further comprising SEQ ID NO:7, SEQ ID NO:8, SEQ ID NO:9 or SEQ ID NO:10.

25. The nucleic acid according to claim 23, further comprising the sequence set forth in SEQ ID NO:7.

26. The nucleic acid according to claim 23, further comprising the sequence set forth in SEQ ID NO:8.

27. The nucleic acid according to claim 23, further comprising the sequence set forth in SEQ ID NO:9.

28. The nucleic acid according to claim 23, comprising the sequence set forth in SEQ ID NO:10.


SEQUENCE LISTING (133 sequences)

SEQ ID NO: 1 - protein, 231 aa, Artificial Sequence
  CDRs of CAB1 protein
XXXXXXXXXXXXXXXXXXXXXXXXXGFNIKDSYMHXXXXXXXXXXXXXXWIDPENGDTEYAPKFQXXXXXXXXXXXXXXXXXXXXXXXXXXXXXXXXXGTPTGPYYFDYXXXXXXXXXXXXXXXXXXXXXXXXXXXXXXXXXXXXXXXXXXXXXXXXXSASSSVSYMHXXXXXXXXXXXXXXXSTSNLASXXXXXXXXXXXXXXXXXXXXXXXXXXXXXXXXQQRSSYPLT

SEQ ID NO: 2 - protein, 605 aa, Artificial Sequence
  CAB1 protein
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 3 - protein, 361 aa, Artificial Sequence
  BLA protein
TPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 4 - protein, 3 aa, Unknown
  skipped
AAA

SEQ ID NO: 5 - protein, 231 aa, Artificial Sequence
  CDRs of CAB1.6 protein variant
XXXXXXXXXXXXXXXXXXXXXXXXXGFNIKDSYMHXXXXXXXXXXXXXXWIDPENGDTEYAPKFQXXXXXXXXXXXXXXXXXXXXXXXXXXXXXXXXXGLPTGPYYFDYXXXXXXXXXXXXXXXXXXXXXXXXXXXXXXXXXXXXXXXXXXXXXXXXXSASSSVSYMHXXXXXXXXXXXXXXXDTSNLASXXXXXXXXXXXXXXXXXXXXXXXXXXXXXXXXQQRDSYPLT

SEQ ID NO: 6 - protein, 231 aa, Artificial Sequence
  CDRs of CAB1.7 protein variant
XXXXXXXXXXXXXXXXXXXXXXXXXGFNIKDSYMHXXXXXXXXXXXXXXWIDPENGDTEYAPKFQXXXXXXXXXXXXXXXXXXXXXXXXXXXXXXXXXGLPLGAIYNDYXXXXXXXXXXXXXXXXXXXXXXXXXXXXXXXXXXXXXXXXXXXXXXXXXSASSAVYAMHXXXXXXXXXXXXXXXDTSNLASXXXXXXXXXXXXXXXXXXXXXXXXXXXXXXXXQQRDSYPLT

SEQ ID NO: 7 - protein, 605 aa, Artificial Sequence
  CAB 1.6 protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPTGPYYFDYWGQGTTVTVSSGGGGSGGGGSGGGGSENVLTQSPAIVSASPGEKVTITCSASSSVSYMHWFQQKPGTSPKLVIYDTSNLASGVPARFSGSGSGTSYSLTISRMEAEDAATYYCQQRDSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 8 - protein, 605 aa, Artificial Sequence
  CAB1.6i protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPTGPYYFDYWGQGTTVTVSSGGGGSGGGGSGGGGSENVLTQSPAIVSASPGEKVTITCSASSSVSYMHWFQQKPGTSPKLVIYDTSNLASGVPARFSGSGSGTSYSLTISRMEAEDAATYYCQQRDSYPLTFGAGTKLELKRAATPVSEKQLAEVVANTITPLMAAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGAYVAFIPEKQIGIVMLANTSYPNPARVEAAYHILEALQ

SEQ ID NO: 9 - protein, 605 aa, Artificial Sequence
  CAB1.7 protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPLGAIYNDYWGQGTTVTVSSGGGGSGGGGSGGGGSENVLTQSPAIVSASPGEKVTITCSASSAVYAMHWFQQKPGTSPKLVIYDTSNLASGVPARFSGSGSGTSYSLTISRMEAEDAATYYCQQRDSYPLTFGAGTKLELDRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 10 - protein, 605 aa, Artificial Sequence
  CAB1.7i protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPLGAIYNDYWGQGTTVTVSSGGGGSGGGGSGGGGSENVLTQSPAIVSASPGEKVTITCSASSAVYAMHWFQQKPGTSPKLVIYDTSNLASGVPARFSGSGSGTSYSLTISRMEAEDAATYYCQQRDSYPLTFGAGTKLELKRAATPVSEKQLAEVVANTITPLMAAQSVPGMAVAVIYQGKPHYYTFGKAKIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGAYVAFIPEKQIGIVMLANTSYPNPARVEAAYHILEALQ

SEQ ID NO: 11 - protein, 244 aa, Artificial Sequence
  CAB1 protein fragment
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAA

SEQ ID NO: 12 - DNA, 5178 bp, Artificial Sequence
  synthetic pME27.1 plasmid sequence
aggaattatcatatgaaatacctgctgccgaccgctgctgctggtctgctgctcctcgctgcccagccggccatggcccaggtgaaactgcagcagtctggggcagaacttgtgaggtcagggacctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggcdcacggtcaccgtctcctcaggtggaggcggttcaggcggaggtggctctggcggtggcggatcagaaaatgtgctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacagtaggaattcgagctccgtcgacaagcttgcggccgcactcgagatcaaacgggctagccagccagaactcgccccggaagaccccgaggatgtcgagcaccaccaccaccaccactgagatccggctgctaacaaagcccgaaaggaagctgagttggctgctgccaccgctgagcaataactagcataaccccttggggcctctaaacgggtcttgaggggtttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgtggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcacgatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagacagatcgctgagataggtgcctcactgattaagcattggtaactgtcagaccaagtttactcatatatactttagattgatttaaaacttcattttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctttctagattaagg

SEQ ID NO: 13 - protein, 120 aa, Artificial Sequence
  CAB1 heavy chain sequence
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 14 - protein, 15 aa, Artificial Sequence
  CAB1 linker sequence
GGGGSGGGGSGGGGS

SEQ ID NO: 15 - protein, 110 aa, Artificial Sequence
  CAB1 light chain sequence
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAT

SEQ ID NO: 16 - protein, 360 aa, Artificial Sequence
  BLA protein fragment
PVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 17 - protein, 605 aa, Artificial Sequence
  SW149.5 protein
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPLGAIYNDYWGQGTTVTVSSGGGGSGGGGSGGGGSENVLTQSPAIVSASPGEKVTITCSASSSVSYMHWFQQKPGTSPKLVIYDTSNLASGVPARFSGSGSGTSYSLTISRMEAEDAATYYCQQRDSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 18 - protein, 605 aa, Artificial Sequence
  CAB1.1 protein variant
QVKLQQSGAELVKSGGSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLVIVSTSNLASGVPARFSGSGSGTSYSLTISRMEAEDAATYYCQQRSSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 19 - DNA, 1815 bp, Artificial Sequence
  CAB1.2 variant coding sequence
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtcgtcacccagtctccagcaatcgtgtctgcatctccaggggagaaggtcaccataacctgcagtgccagctcaagtgtaagttacatgcactggttccagcagaagccaggcacttctcccaaactcgtgatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatcgggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 20 - protein, 605 aa, Artificial Sequence
  CAB1.2 protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIVSASPGEKVTITCSASSSVSYMHWFQQKPGTSPKLVIYSTSNLASGVPARFSGSGSGTSYSLTISRMEAEDAATYYCQQRSSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 21 - protein, 231 aa, Artificial Sequence
  CDRs of CAB1.4 protein variant
XXXXXXXXXXXXXXXXXXXXXXXXXGFNIKDSYMHXXXXXXXXXXXXXXWIDPENGDTEYAPKFQXXXXXXXXXXXXXXXXXXXXXXXXXXXXXXXXXGTPTGPYYFDYXXXXXXXXXXXXXXXXXXXXXXXXXXXXXXXXXXXXXXXXXXXXXXXXXSASSSVSYMHXXXXXXXXXXXXXXXDTSNLASXXXXXXXXXXXXXXXXXXXXXXXXXXXXXXXXQQRDSYPLT

SEQ ID NO: 22 - DNA, 771 bp, Artificial Sequence
  sequence encoding CDRs of CAB1.4 variant
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttcaacattaaagactcctatatgcacnnnnnnnnnnnnnnnnnnnnnnnnnnnnnnnnnnnnnnnnnntggattgatcctgagaatggtgatactgaatatgccccgaagttccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggactccgactgggccgtactactttgactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtgccagctcaagtgtaagttacatgcacnnnnnnnnnnnnnnnnnnnnnnnnnnnnnnnnnnnnnnnnnnnnngatacatccaacctggcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagcaaagagatagttacccactcacg

SEQ ID NO: 23 - DNA, 1815 bp, Artificial Sequence
  sequence encoding CAB1.4 variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcaagtgtaagttacatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcgctggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 24 - protein, 605 aa, Artificial Sequence
  CAB1.4 protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIVSASPGEKVTITCSASSSVSYMHWFQQKPGTSPKLVIYDTSNLASGVPARFSGSGSGTSYSLTISRMEAEDAATYYCQQRDSYPLTFGAGTKLELKRAATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 25 - DNA, 771 bp, Artificial Sequence
  sequence encoding CDRs of CAB1.6 variant
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttcaacattaaagactcctatatgcacnnnnnnnnnnnnnnnnnnnnnnnnnnnnnnnnnnnnnnnnnntggattgatcctgagaatggtgatactgaatatgccccgaagttccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggctcccgactgggccgtactactttgactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtgccagctcaagtgtaagttacatgcacnnnnnnnnnnnnnnnnnnnnnnnnnnnnnnnnnnnnnnnnnnnnngatacatccaacctggcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagcaaagagatagttacccactcacg

SEQ ID NO: 26 - DNA, 1815 bp, Artificial Sequence
  sequence encoding CAB1.6 protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgactgggccgtactactttgactactggggccaagggaccacggtcaccgtctcctcaggtggaggcggttcaggcggaggtggctctggcggtggcggatcagaaaatgtcgtcacccagtctccagcaatcgtgtctgcatctccaggggagaaggtcaccataacctgcagtgccagctcaagtgtaagttacatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 27 - DNA, 1815 bp, Artificial Sequence
  sequence encoding CAB1.6i protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgactgggccgtactactttgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcaagtgtaacttacatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatggcggcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgccctgggcagaagtgaatccaccggctcccccggtcaaagcgtcctgggtccataaaacgggctctactggcgggtttggcgcgtacgtggcctttattcctgaaaagcagatcggtattgtgatgctcgcgaatacaagctatccgaacccggcacgcgttgaggcggcataccatatcctcgaggcgctacag

SEQ ID NO: 28 - DNA, 771 bp, Artificial Sequence
  sequence encoding CDRs of CAB1.7 protein variant
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttcaacattaaagactcctatatgcacnnnnnnnnnnnnnnnnnnnnnnnnnnnnnnnnnnnnnnnnnntggattgatcctgagaatggtgatactgaatatgccccgaagttccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggctcccgctcggggccatttacaacgactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtgccagctcagctgtatatgccatgcacnnnnnnnnnnnnnnnnnnnnnnnnnnnnnnnnnnnnnnnnnnnnngatacatccaacctggcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagcaaagagatagttacccactcacg

SEQ ID NO: 29 - DNA, 1815 bp, Artificial Sequence
  sequence encoding CAB1.7 protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgctcggggccatttacaacgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcagctgtatatgccatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 30 - DNA, 1815 bp, Artificial Sequence
  sequence encoding CAB1.7i protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgctcggggccatttacaacgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcagctgtatatgccatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatggcggcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcgcgtacgtggcctttattcctgaaaagcagatcggtattgtgatgctcgcgaatacaagctatccgaacccggcacgcgttgaggcggcataccatatcctcgaggcgctacag

SEQ ID NO: 31 - DNA, 771 bp, Artificial Sequence
  sequence encoding CDRs of CAB1 protein
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttcaacattaaagactcctatatgcacnnnnnnnnnnnnnnnnnnnnnnnnnnnnnnnnnnnnnnnnnntggattgatcctgagaatggtgatactgaatatgccccgaagttccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggactccgactgggccgtactactttgactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtgccagctcaagtgtaagttacatgcacnnnnnnnnnnnnnnnnnnnnnnnnnnnnnnnnnnnnnnnnnnnnnagcacatccaacctggcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagcaaagatctagttacccactcacg

SEQ ID NO: 32 - DNA, 1815 bp, Artificial Sequence
  sequence encoding CAB1 protein
caggtgaaactgcagcagtctggggcagaacttgtgaggtcagggacctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgcggatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 33 - protein, 231 aa, Artificial Sequence
  CDRs of SW149.5 protein
XXXXXXXXXXXXXXXXXXXXXXXXXGFNIKDSYMHXXXXXXXXXXXXXXWIDPENGDTEYAPKFQXXXXXXXXXXXXXXXXXXXXXXXXXXXXXXXXXGLPLGAIYNDYXXXXXXXXXXXXXXXXXXXXXXXXXXXXXXXXXXXXXXXXXXXXXXXXXSASSSVSYMHXXXXXXXXXXXXXXXDTSNLASXXXXXXXXXXXXXXXXXXXXXXXXXXXXXXXXQQRDSYPLT

SEQ ID NO: 34 - DNA, 771 bp, Artificial Sequence
  sequence encoding CDRs of SW149.4 protein
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttcaacattaaagactcctatatgcacnnnnnnnnnnnnnnnnnnnnnnnnnnnnnnnnnnnnnnnnnntggattgatcctgagaatggtgatactgaatatgccccgaagttccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggctcccgctcggggccatttacaacgactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtgccagctcaagtgtaagttacatgcacnnnnnnnnnnnnnnnnnnnnnnnnnnnnnnnnnnnnnnnnnnnnngatacatccaacctggcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagcaaagagatagttacccactcacg

SEQ ID NO: 35 - DNA, 1815 bp, Artificial Sequence
  sequence encoding SW149.5 protein
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgctcggggccatttacaacgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcaagtgtaagttacatgcactggttccagcagaagccaggcacttctcccaaactcgtgatttatgatacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 36 - DNA, 1083 bp, Artificial Sequence
  sequence encoding BLA protein
acaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 37 - DNA, 1815 bp, Artificial Sequence
  sequence encoding CAB1.1 protein variant
caggtgaaactgcagcagtctggggcagaacttgtgaaatcagggggctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcatgtctgcatctccaggggagaaggtcaccataacctgcagtgccagctcaagtgtaagttacatgcactggttccagcagaagccaggcacttctcccaaactcgtgatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacgggcggccacaccggtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctgagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacag

SEQ ID NO: 38 - protein, 605 aa, Artificial Sequence
  CAB1.2i protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIVSASPGEKVTITCSASSSVSYMHWFQQKPGTSPKLVIYSTSNLASGVPARFSGSGSGTSYSLTISRMEAEDAATYYCQQRSSYPLTFGAGTKLELKRAATPVSEKQLAEVVANTITPLMAAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGAYVAFIPEKQIGIVMLANTSYPNPARVEAAYHILEALQ

SEQ ID NO: 39 - DNA, 1815 bp, Artificial Sequence
  sequence encoding CAB1.2i protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcaagtgtaagttacatgcactggttccagcagaagccaggcacttctcccaaactcgtgatttatagcacatccaacctggcttctggagtccctgctcgcttcagtggcagtggatctgggacctcttactctctcacaatcagccgaatggaggctgaagatgctgccacttattactgccagcaaagatctagttacccactcacgttcggtgctggcaccaagctggagctgaaacggggcgccacaccggtgtcagaaaaacagctggcggaggtggtcgcgaatacgattaccccgctgatggcggcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactgccgccgttgcccgtggcagaagtgaatccaccggctcccccggtcaaagcgtcctgggtccataaaacgggctctactggcgggtttggcgcgtacgtggcctttattcctgaaaagcagatcggtattgtgatgctcgcgaatacaagctatccgaacccggcacgcgttgaggcggcataccatatcctcgaggcgctacag

SEQ ID NO: 40 - protein, 605 aa, Artificial Sequence
  CAB1.13i protein variant
QVQLQQSGAELVKSGGSVKLSCTASGFNIKDSYMHWVRQGPEQGLEWIGWIDPENGDTEYAPKFQGKATFTTDTSSNTAYLQLSSLTSEDTAVYYCNEGLPLGAIYNDYWGQGTTVTVSSGGGGSGGGGSGGGGSENVLTQSPAIVSASPGEKVTITCSASSAVYAMHWFQQKPGTSPKLVIYSTSNLASGVPARFSGSGSGTSYSLTISRMEAEDAATYYCQQRDSYPLTFGAGTKLELKRAATPVSEKQLAEVVANTITPLMAAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGAYVAFIPEKQIGIVMLANTSYPNPARVEAAYHILEALQ

SEQ ID NO: 41 - DNA, 1814 bp, Artificial Sequence
  sequence encoding CAB1.13i protein variant
caggtgcagctgcagcagtctggggcagaacttgtgaaatcagggggctcagtcaagttgtcctgcacagcttctggcttcaacattaaagactcctatatgcactgggtgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggctcccgctcggggccatttacaacgactactggggccaagggaccacggtcaccgtctcctcaggtggaggcggttcaggcggaggtggctctggcggtggcggatcagaaaatgtgctcacccagtctccagcaatcgtgtctgcatctccaggggagaaggtcaccataacctgcagtgccagctcagctgtatatgccatgcactggttccagcagaagccaggcacttctcccaaactcgtgatttatagcacatccaacctggcttctggagtccctgctcgcttcagtggcagtggatctgggacctcttactctctcacaatcagccgaatggaggctgaagatgctgccacttattactgccagcaaagagatagttacccactcacgttcggtgctggcaccaagctggagctgaaacgggcggccacaccggtgtcagaaaaacagctggcggaggtggtcgcgaatacgattaccccgctgatggcggcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcgcgtacgtggcctttattcctgaaaagcagatcggtattgtgatgctcgcgaatacaagctatccgaacccggcacgcgttgaggcggcataccatatcctcgaggcgctacag

SEQ ID NO: 42 - protein, 623 aa, Artificial Sequence
  CAB1.13i protein variant
DIVLTQSPASLSVSLGQRATMSCRAGESVDIFGVGFLHWYQQKPGQPPKLLIYRASNLESGIPVRFSGTGSGTDFTLIIDPVEADDVATYYCQQTNEDPYTFGGGTKLEIKGGGGSGGGGSGGGGSGGGGSGGGGSGGGGSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSTPVSEKQLAEVVANTITPLMAAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGAYVAFIPEKQIGIVMLANTSYPNPARVEAAYHILEALQ

SEQ ID NO: 43 - DNA, 1869 bp, Artificial Sequence
  sequence encoding CAB1.11i protein variant
gacatcgtcctgacccagagcccggcaagcctgtctgtttccctgggccagcgtgccactatgtcctgcagagcgggtgagtctgttgacattttcggtgtcggttttctgcactggtaccaacagaaaccgggtcagccgccaaaactgctgatctatcgtgcttctaacctggagtccggcatcccggtacgtttctccggtactggctctggtactgattttaccctgattatcgacccggtggaagcagacgatgttgccacctactattgccagcagaccaacgaggatccgtacaccttcggtggcggtactaaactggagatcaaaggcggtggtggttctggtggtggtggtagcggtggcggtggtagcggtggcggtggcagcggtggtggtggctctggtggcggtggctctgaagtgcagctgcagcagtccggtgcggagctcgttgaaccgggcgcttctgtgaaactgtcttgcactgcatctggtttcaacattaaggacacctacatgcactgggtgaaacaacgcccggaacagggtctggagtggatcggtcgcatcgatccggctaacggtaacagcaaatacgtgccaaaattccagggtaaagcaaccatcactgctgatacctcctctaacactgcttacctgcagctgacttccctgactagcgaagacaccgcggtttattactgcgctccgttcggctactatgtcagcgattacgcaatggcctactggggtcagggcacctctgttaccgtttctagcacaccggtgtcagaaaaacagctggcggaggtggtcgcgaatacgattaccccgctgatggcggcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcgcgtacgtggcctttattcctgaaaagcagatcggtattgtgatgctcgcgaatacaagctatccgaacccggcacgcgttgaggcggcataccatatcctcgaggcgctacag

SEQ ID NO: 44 - DNA, 37 bp, Artificial Sequence
  synthetic oligonucleotide
cggccatggcccaggtgcagctgcagcagtctggggc

SEQ ID NO: 45 - DNA, 37 bp, Artificial Sequence
  synthetic oligonucleotide
ctggggcagaacttgtgaaatcagggacctcagtcaa

SEQ ID NO: 46 - DNA, 37 bp, Artificial Sequence
  synthetic oligonucleotide
gggcagaacttgtgaggccggggacctcagtcaagtt

SEQ ID NO: 47 - DNA, 37 bp, Artificial Sequence
  synthetic oligonucleotide
aacttgtgaggtcagggggctcagtcaagttgtcctg

SEQ ID NO: 48 - DNA, 37 bp, Artificial Sequence
  synthetic oligonucleotide
gcacagcttctggcttcaccattaaagactcctatat

SEQ ID NO: 49 - DNA, 37 bp, Artificial Sequence
  synthetic oligonucleotide
cagcttctggcttcaactttaaagactcctatatgca

SEQ ID NO: 50 - DNA, 37 bp, Artificial Sequence
  synthetic oligonucleotide
cttctggcttcaacattagcgactcctatatgcactg

SEQ ID NO: 51 - DNA, 37 bp, Artificial Sequence
  synthetic oligonucleotide
actcctatatgcactgggtgaggcaggggcctgaaca

SEQ ID NO: 52 - DNA, 37 bp, Artificial Sequence
  synthetic oligonucleotide
tgcactggttgaggcaggcgcctgaacagggcctgga

SEQ ID NO: 53 - DNA, 37 bp, Artificial Sequence
  synthetic oligonucleotide
ggttgaggcaggggcctggccagggcctggagtggat

SEQ ID NO: 54 - DNA, 37 bp, Artificial Sequence
  synthetic oligonucleotide
ccccgaagttccagggccgtgccacttttactacaga

SEQ ID NO: 55 - DNA, 37 bp, Artificial Sequence
  synthetic oligonucleotide
cgaagttccagggcaagttcacttttactacagacac

SEQ ID NO: 56 - DNA, 37 bp, Artificial Sequence
  synthetic oligonucleotide
tccagggcaaggccactattactacagacacatcctc

SEQ ID NO: 57 - DNA, 37 bp, Artificial Sequence
  synthetic oligonucleotide
gcaaggccacttttactcgcgacacatcctccaacac

SEQ ID NO: 58 - DNA, 37 bp, Artificial Sequence
  synthetic oligonucleotide
ttactacagacacatccaaaaacacagcctacctgca

SEQ ID NO: 59 - DNA, 37 bp, Artificial Sequence
  synthetic oligonucleotide
ctgccgtctattattgtgcggaggggactccgactgg

SEQ ID NO: 60 - DNA, 37 bp, Artificial Sequence
  synthetic oligonucleotide
ccgtctattattgtaatcgcgggactccgactgggcc

SEQ ID NO: 61 - DNA, 37 bp, Artificial Sequence
  synthetic oligonucleotide
ctggcggtggcggatcacagaatgtgctcacccagtc

SEQ ID NO: 62 - DNA, 37 bp, Artificial Sequence
  synthetic oligonucleotide
gcggtggcggatcagaaagcgtgctcacccagtctcc

SEQ ID NO: 63 - DNA, 38 bp, Artificial Sequence
  synthetic oligonucleotide
gaaaatgtgctcacccagccgccagcaatcatgtctgc

SEQ ID NO: 64 - DNA, 37 bp, Artificial Sequence
  synthetic oligonucleotide
tgctcacccagtctccaagcatcatgtctgcatctcc

SEQ ID NO: 65 - DNA, 37 bp, Artificial Sequence
  synthetic oligonucleotide
cccagtctccagcaatcgtgtctgcatctccagggga

SEQ ID NO: 66 - DNA, 37 bp, Artificial Sequence
  synthetic oligonucleotide
tgtctgcatctccagggcagaaggtcaccataacctg

SEQ ID NO: 67 - DNA, 37 bp, Artificial Sequence
  synthetic oligonucleotide
ctgcatctccaggggagaccgtcaccataacctgcag

SEQ ID NO: 68 - DNA, 37 bp, Artificial Sequence
  synthetic oligonucleotide
taagttacatgcactggtaccagcagaagccaggcac

SEQ ID NO: 69 - DNA, 37 bp, Artificial Sequence
  synthetic oligonucleotide
gcacttctcccaaactcgtgatttatagcacatccaa

SEQ ID NO: 70 - DNA, 37 bp, Artificial Sequence
  synthetic oligonucleotide
tggcttctggagtccctgatcgcttcagtggcagtgg

SEQ ID NO: 71 - DNA, 37 bp, Artificial Sequence
  synthetic oligonucleotide
ctcgcttcagtggcagtaaatctgggacctcttactc

SEQ ID NO: 72 - DNA, 37 bp, Artificial Sequence
  synthetic oligonucleotide
gtggatctgggacctctgcgtctctcacaatcagccg

SEQ ID NO: 73 - DNA, 37 bp, Artificial Sequence
  synthetic oligonucleotide
ctctcacaatcagccgactggaggctgaagatgctgc

SEQ ID NO: 74 - DNA, 37 bp, Artificial Sequence
  synthetic oligonucleotide
gaatggaggctgaagatgaagccacttattactgcca

SEQ ID NO: 75 - DNA, 37 bp, Artificial Sequence
  synthetic oligonucleotide
aggctgaagatgctgccgattattactgccagcaaag

SEQ ID NO: 76 - DNA, 37 bp, Artificial Sequence
  synthetic oligonucleotide
acccactcacgttcggtggcggcaccaagctggagct

SEQ ID NO: 77 - DNA, 37 bp, Artificial Sequence
  primer
cttctggcttcaacattsatgactcctatatgcactg

SEQ ID NO: 78 - DNA, 37 bp, Artificial Sequence
  primer
ctggcttcaacattaaasattcctatatgcactgggt

SEQ ID NO: 79 - DNA, 37 bp, Artificial Sequence
  primer
gcttcaacattaaagacsattatatgcactgggtgag

SEQ ID NO: 80 - DNA, 37 bp, Artificial Sequence
  primer
tcaacattaaagactccsatatgcactgggtgaggca

SEQ ID NO: 81 - DNA, 37 bp, Artificial Sequence
  primer
ttaaagactcctatatgsattgggtgaggcaggggcc

SEQ ID NO: 82 - DNA, 37 bp, Artificial Sequence
  primer
gcctggagtggattggasatattgatcctgagaatgg

SEQ ID NO: 83 - DNA, 37 bp, Artificial Sequence
  primer
agtggattggatggattsatcctgagaatggtgatac

SEQ ID NO: 84 - DNA, 37 bp, Artificial Sequence
  primer
ttggatggattgatcctsataatggtgatactgaata

SEQ ID NO: 85 - DNA, 37 bp, Artificial Sequence
  primer
gatggattgatcctgagsatggtgatactgaatatgc

SEQ ID NO: 86 - DNA, 37 bp, Artificial Sequence
  primer
ttgatcctgagaatggtsatactgaatatgccccgaa

SEQ ID NO: 87 - DNA, 37 bp, Artificial Sequence
  primer
atcctgagaatggtgatsatgaatatgccccgaagtt

SEQ ID NO: 88 - DNA, 37 bp, Artificial Sequence
  primer
ctgagaatggtgatactsattatgccccgaagttcca

SEQ ID NO: 89 - DNA, 37 bp, Artificial Sequence
  primer
gtgatactgaatatgccsataagttccagggcaaggc

SEQ ID NO: 90 - DNA, 37 bp, Artificial Sequence
  primer
atactgaatatgccccgsatttccagggcaaggccac

SEQ ID NO: 91 - DNA, 37 bp, Artificial Sequence
  primer
aatatgccccgaagttcsatggcaaggccacttttac

SEQ ID NO: 92 - DNA, 37 bp, Artificial Sequence
  primer
ccgtctattattgtaatsatgggactccgactgggcc

SEQ ID NO: 93 - DNA, 37 bp, Artificial Sequence
  primer
tctattattgtaatgagsatactccgactgggccgta

SEQ ID NO: 94 - DNA, 37 bp, Artificial Sequence
  primer
attattgtaatgaggggsatccgactgggccgtacta

SEQ ID NO: 95 - DNA, 37 bp, Artificial Sequence
  primer
attgtaatgaggggactsatactgggccgtactactt

SEQ ID NO: 96 - DNA, 37 bp, Artificial Sequence
  primer
gtaatgaggggactccgsatgggccgtactactttga

SEQ ID NO: 97 - DNA, 37 bp, Artificial Sequence
  primer
atgaggggactccgactsatccgtactactttgacta

SEQ ID NO: 98 - DNA, 37 bp, Artificial Sequence
  primer
aggggactccgactgggsattactactttgactactg

SEQ ID NO: 99 - DNA, 37 bp, Artificial Sequence
  primer
ctccgactgggccgtacsattttgactactggggcca

SEQ ID NO: 100 - DNA, 37 bp, Artificial Sequence
  primer
taacctgcagtgccagcsatagtgtaagttacatgca

SEQ ID NO: 101 - DNA, 37 bp, Artificial Sequence
  primer
cctgcagtgccagctcasatgtaagttacatgcactg

SEQ ID NO: 102 - DNA, 37 bp, Artificial Sequence
  primer
gcagtgccagctcaagtsatagttacatgcactggtt

SEQ ID NO: 103 - DNA, 37 bp, Artificial Sequence
  primer
gtgccagctcaagtgtasattacatgcactggttcca

SEQ ID NO: 104 - DNA, 37 bp, Artificial Sequence
  primer
ccagctcaagtgtaagtsatatgcactggttccagca

SEQ ID NO: 105 - DNA, 37 bp, Artificial Sequence
  primer
ctcccaaactcgtgattsatagcacatccaacctggc

SEQ ID NO: 106 - DNA, 37 bp, Artificial Sequence
  primer
ccaaactcgtgatttatsatacatccaacctggcttc

SEQ ID NO: 107 - DNA, 37 bp, Artificial Sequence
  primer
aactcgtgatttatagcsattccaacctggcttctgg

SEQ ID NO: 108 - DNA, 37 bp, Artificial Sequence
  primer
tcgtgatttatagcacasataacctggcttctggagt

SEQ ID NO: 109 - DNA, 37 bp, Artificial Sequence
  primer
tgatttatagcacatccsatctggcttctggagtccc

SEQ ID NO: 110 - DNA, 37 bp, Artificial Sequence
  primer
atagcacatccaacctgsattctggagtccctgctcg

SEQ ID NO: 111 - DNA, 37 bp, Artificial Sequence
  primer
gcacatccaacctggctsatggagtccctgctcgctt

SEQ ID NO: 112 - DNA, 37 bp, Artificial Sequence
  primer
cttattactgccagcaasattctagttacccactcac

SEQ ID NO: 113 - DNA, 36 bp, Artificial Sequence
  primer
attactgccagcaaagasatagttacccactcacgt

SEQ ID NO: 114 - DNA, 36 bp, Artificial Sequence
  primer
actgccagcaaagatctsattacccactcacgttcg

SEQ ID NO: 115 - DNA, 36 bp, Artificial Sequence
  primer
gccagcaaagatctagtsatccactcacgttcggtg

SEQ ID NO: 116 - DNA, 37 bp, Artificial Sequence
  primer
aaagatctagttacccasatacgttcggtgctggcac

SEQ ID NO: 117 - DNA, 17 bp, Artificial Sequence
  primer
caggaaacagctatgac

SEQ ID NO: 118 - DNA, 22 bp, Artificial Sequence
  primer
ggaccacggtcaccgtctcctc

SEQ ID NO: 119 - DNA, 37 bp, Artificial Sequence
  synthetic oligonucleotide
attattgtaatgaggggnnsccgactgggccgtacta

SEQ ID NO: 120 - DNA, 37 bp, Artificial Sequence
  synthetic oligonucleotide
tagtacggcccagtcggsnncccctcattacaataat

SEQ ID NO: 121 - DNA, 37 bp, Artificial Sequence
  primer
gtaatgaggggctgccgnnsgggccgtactactttga

SEQ ID NO: 122 - DNA, 37 bp, Artificial Sequence
  primer
tcaaagtagtacggcccsnncggcagcccctcattac

SEQ ID NO: 123 - DNA, 37 bp, Artificial Sequence
  primer
cgactgggccgtactacnnsgactactggggccaagg

SEQ ID NO: 124 - DNA, 37 bp, Artificial Sequence
  primer
ccttggccccagtagtcsnngtagtacggcccagtcg

SEQ ID NO: 125 - DNA, 47 bp, Artificial Sequence
  primer
gaggggctcccgctcgggrvcntttacaacgactactggggccaagg

SEQ ID NO: 126 - DNA, 47 bp, Artificial Sequence
  primer
ccttggccccagtagtcgttgtaaangbycccgagcgggagcccctc

SEQ ID NO: 127 - DNA, 37 bp, Artificial Sequence
  primer
cttctggcttcaacattaccgactcctatatgcactg

SEQ ID NO: 128 - DNA, 36 bp, Artificial Sequence
  primer
gcctggagtggattggatttattgatcctgagaatg

SEQ ID NO: 129 - DNA, 59 bp, Artificial Sequence
  primer
gatcctgagaatggtswtrctgaatatgcccbgaagttcrncggcaaggccacttttac

SEQ ID NO: 130 - DNA, 44 bp, Artificial Sequence
  primer
ctgcagtgccagctcadctgtaymtdccatgcactggttccagc

SEQ ID NO: 131 - DNA, 49 bp, Artificial Sequence
  primer
cgtgatttatgatacarvcaacctggctrstggagtccctgctcgcttc

SEQ ID NO: 132 - DNA, 35 bp, Artificial Sequence
  primer
gattaccccgctgatggcggcccagtctgttccag

SEQ ID NO: 133 - DNA, 39 bp, Artificial Sequence
  primer
ctactggcgggtttggcgcgtacgtggcctttattcctg